SEQUENCE LISTING (10 sequences)

SEQ ID NO: 1 - protein, 5 aa, synthetic construct
SYAIS

SEQ ID NO: 2 - protein, 17 aa, synthetic construct
GIIPIFDTANYAQKFQG

SEQ ID NO: 3 - protein, 14 aa, synthetic construct
PGLAAAYDTGSLDY

SEQ ID NO: 4 - protein, 11 aa, synthetic construct
RASQSVRSYLA

SEQ ID NO: 5 - protein, 7 aa, synthetic construct
DASNRAT

SEQ ID NO: 6 - protein, 9 aa, synthetic construct
QQRNYWPLT

SEQ ID NO: 7 - protein, 123 aa, synthetic construct
QVQLVQSGAEVKKPGSSVKVSCKASGGTFSSYAISWVRQAPGQGLEWMGGIIPIFDTANYAQKFQGRVTITADESTSTAYMELSSLRSEDTAVYYCARPGLAAAYDTGSLDYWGQGTLVTVSS

SEQ ID NO: 8 - protein, 107 aa, synthetic construct
EIVLTQSPATLSLSPGERATLSCRASQSVRSYLAWYQQKPGQAPRLLIYDASNRATGIPARFSGSGSGTDFTLTISSLEPEDFAVYYCQQRNYWPLTFGQGTKVEIK

SEQ ID NO: 9 - protein, 450 aa, synthetic construct
QVQLVQSGAEVKKPGSSVKVSCKASGGTFSSYAISWVRQAPGQGLEWMGGIIPIFDTANYAQKFQGRVTITADESTSTAYMELSSLRSEDTAVYYCARPGLAAAYDTGSLDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 10 - protein, 214 aa, synthetic construct
EIVLTQSPATLSLSPGERATLSCRASQSVRSYLAWYQQKPGQAPRLLIYDASNRATGIPARFSGSGSGTDFTLTISSLEPEDFAVYYCQQRNYWPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2026/0144809 A1
(43) Pub. Date: May 28, 2026

(54) METHODS OF TREATING BACILLUS CALMETTE-GUÉRIN (BCG)-NAÏVE HIGH-RISK NON-MUSCLE INVASIVE BLADDER CANCER (HR-NMIBC)

(71) Applicant: TARIS Biomedical LLC, Lexington, MA (US)

(72) Inventors: Christopher CUTIE, Lexington, MA (US); Kiran PATEL, Chesterbrook, PA (US); Hussein SWEITI, Royersford, PA (US)

(21) Appl. No.: 19/327,753

(22) Filed: Sep. 12, 2025

Related U.S. Application Data
(60) Provisional application No. 63/694,756, filed on Sep. 13, 2024.
(60) Provisional application No. 63/705,433, filed on Oct. 9, 2024.

(51) Int. Cl. A61K 31/7068; A61K 9/00; A61K 39/00; A61K 39/395; A61P 35/00

(52) U.S. Cl. CPC A61K 31/7068; A61K 9/0019; A61K 9/0034; A61K 39/3955; A61P 35/00; A61K 2039/54; A61K 2039/545

(57) ABSTRACT

The present disclosure relates generally to methods of treating high-risk non-muscle invasive bladder cancer (HR-NMIBC), and more specifically to methods of treating Bacillus Calmette-Guérin (BCG)-naïve HR-NMIBC in individuals by administering intravesical gemcitabine.

### CROSS-REFERENCE TO RELATED APPLICATIONS

This application claims priority to and the benefit of U.S. Provisional Application Nos. 63/694,756, filed Sep. 13, 2024; and 63/705,433, filed Oct. 9, 2024, the contents of each of which are incorporated herein by reference in their entirety.

### FIELD

The present disclosure relates generally to methods of treating high-risk non-muscle invasive bladder cancer (HR-NMIBC), and more specifically to methods of treating Bacillus Calmette-Guérin (BCG)-naïve HR-NMIBC in individuals by administering intravesical gemcitabine.

### REFERENCE TO AN ELECTRONIC SEQUENCE LISTING

The contents of the electronic sequence listing (761662003400SEQLIST.xml; Size: 10,458 bytes; and Date of Creation: Sep. 12, 2025) is herein incorporated by reference in its entirety.

### BACKGROUND

Bladder cancer is a significant medical problem, and currently available treatment options are unsatisfactory for a number of reasons. The mainstay of treatment for HR-NMIBC is transurethral resection of bladder tumor (TURBT), biopsy only for patients with carcinoma in-situ, CIS, followed by intravesical treatment with BCG. While BCG therapy can be successful at preventing early tumor recurrences in patients with high-risk non-muscle invasive bladder cancer (HR-NMIBC), most patients do not maintain sustained remissions (Shelley et al. Cochrane Database Syst Rev. 2000; (4):CD001986). BCG treatment eventually fails in up to 50% of patients with almost 50% of these failures occurring within the first 6 months (Lightfoot et al. Scientific World J. 2011 Mar. 7; 11:602-613). Thus, many patients will ultimately develop BCG-unresponsive disease.

Primary radical cystectomy (RC) is another treatment option for some patients. However, the utility of upfront RC in this disease in head-to-head studies with BCG demonstrated that RC is poorly adopted (Catto et al. European Urology. 2021; 79; 5:621-632).

Clinical studies are currently underway assessing the efficacy of systemic checkpoint inhibitors in combination with various schedules of intravesical BCG to extend disease-free and recurrence-free survival (NCT03528694, NCT03799835, NCT04165317). Such studies employ adjuvant designs in patients with HR-NMIBC, adding systemic immunomodulatory therapies to standard induction BCG schedules, with or without maintenance BCG treatment. Such designs rely upon BCG as backbone therapy, and in many cases, employ induction BCG schedules only. In addition, these designs may be subject to overlapping local and systemic toxicity, as well as persistent supply issues related to limited BCG access across all arms (Decaestecker et al. Research and Reports in Urology. 2015; 7:157-163).

Thus, there remains a significant unmet need for alternative therapies in the treatment of patients with BCG-naïve HR-NMIBC. The present application addresses these and other needs.

### SUMMARY

Described herein are methods of treating Bacillus Calmette-Guérin (BCG)-naïve high-risk non-muscle invasive bladder cancer (HR-NMIBC) in individuals by administering intravesical gemcitabine to the bladder. In some aspects, the methods for treating BCG-naïve HR-NMIBC further include intravenously or subcutaneously administering cetrelimab in combination with the intravesical gemcitabine. The gemcitabine can be administered using an intravesical drug delivery system that comprises about 225 mg gemcitabine (or about 256 mg gemcitabine HCL) and provides a controlled release of the gemcitabine into urine while indwelling in the bladder. The indwelling nature of the intravesical drug delivery system allows for extended intravesical delivery of therapeutic gemcitabine concentrations over many days during the indwelling period of the system, resulting in greater tolerability and a potentially enhanced therapeutic effect compared to traditional intravesical gemcitabine instillations, and, more generally, to the standard of care (SOC) for HR-NMIBC.

In some aspects, described herein are methods of treating Bacillus Calmette-Guérin (BCG)-naïve high-risk non-muscle invasive bladder cancer (HR-NMIBC) in an individual, comprising: inserting an intravesical drug delivery system comprising about 225 mg of gemcitabine into the bladder of the individual about once every three weeks (Q3W) for a total of up to six months following starting treatment, wherein each intravesical drug delivery system is removed from the bladder about three weeks after insertion into the bladder and wherein each intravesical drug delivery system is removed prior to insertion of another intravesical drug delivery system.

In some aspects, described herein are methods of treating Bacillus Calmette-Guérin (BCG)-naïve high-risk non-muscle invasive bladder cancer (HR-NMIBC) in an individual, comprising: inserting an intravesical drug delivery system comprising about 256 mg of gemcitabine HCL (equivalent to about 225 mg gemcitabine free base) into the bladder of the individual about once every three weeks (Q3W) for a total of up to six months following starting treatment, wherein each intravesical drug delivery system is removed from the bladder about three weeks after insertion into the bladder and wherein each intravesical drug delivery system is removed prior to insertion of another intravesical drug delivery system.

In some aspects, described herein are methods of increasing event-free survival (EFS) for Bacillus Calmette-Guérin (BCG)-naïve high-risk non-muscle invasive bladder cancer (HR-NMIBC) in an individual, comprising: inserting an intravesical drug delivery system comprising about 225 mg of gemcitabine into the bladder of the individual about once every three weeks (Q3W) for a total of up to six months following starting treatment, wherein each intravesical drug delivery system is removed from the bladder about three weeks after insertion into the bladder and wherein each intravesical drug delivery system is removed prior to insertion of another intravesical drug delivery system, and wherein the methods result in an increased event-free survival (EFS) of the individual compared to the EFS achieved by intravesical BCG, and/or wherein the methods result in an increased EFS in a population of patients receiving such therapy compared to a population of patients receiving intravesical BCG.

In some aspects, described herein are methods of increasing event-free survival (EFS) for Bacillus Calmette-Guérin (BCG)-naïve high-risk non-muscle invasive bladder cancer (HR-NMIBC) in an individual, comprising: inserting an intravesical drug delivery system comprising about 256 mg of gemcitabine HCl (equivalent to about 225 mg gemcitabine free base) into the bladder of the individual about once every three weeks (Q3W) for a total of up to six months following starting treatment, wherein each intravesical drug delivery system is removed from the bladder about three weeks after insertion into the bladder and wherein each intravesical drug delivery system is removed prior to insertion of another intravesical drug delivery system, and wherein the methods result in an increased event-free survival (EFS) of the individual compared to the EFS achieved by intravesical BCG, and/or wherein the methods result in an increased EFS in a population of patients receiving such therapy compared to a population of patients receiving intravesical BCG.

In some aspects, described herein are methods of treating Bacillus Calmette-Guérin (BCG)-naïve high-risk non-muscle invasive bladder cancer (HR-NMIBC) in an individual, comprising: inserting an intravesical drug delivery system comprising about 225 mg of gemcitabine into the bladder of the individual about once every three weeks (Q3W) for a total of up to six months following starting treatment, followed by inserting an intravesical drug delivery system comprising about 225 mg of gemcitabine into the bladder of the individual about once every 12 weeks (Q12W) for up to an additional 18 months after the first six months of treatment, wherein each intravesical drug delivery system is removed from the bladder about three weeks after insertion into the bladder and wherein each intravesical drug delivery system is removed prior to insertion of another intravesical drug delivery system.

In some aspects, described herein are methods of treating Bacillus Calmette-Guérin (BCG)-naïve high-risk non-muscle invasive bladder cancer (HR-NMIBC) in an individual, comprising: inserting an intravesical drug delivery system comprising about 256 mg of gemcitabine HCL (equivalent to about 225 mg gemcitabine free base) into the bladder of the individual about once every three weeks (Q3W) for a total of up to six months following starting treatment, followed by inserting an intravesical drug delivery system comprising about 256 mg of gemcitabine HCL (equivalent to about 225 mg gemcitabine free base) into the bladder of the individual about once every 12 weeks (Q12W) for up to an additional 18 months after the first six months of treatment, wherein each intravesical drug delivery system is removed from the bladder about three weeks after insertion into the bladder and wherein each intravesical drug delivery system is removed prior to insertion of another intravesical drug delivery system.

In some aspects, described herein are methods of treating Bacillus Calmette-Guérin (BCG)-naïve high-risk non-muscle invasive bladder cancer (HR-NMIBC) in an individual, comprising: intravenously administering to the individual about 360 mg of cetrelimab about once every three weeks (Q3W) for at least six months following starting treatment; and inserting an intravesical drug delivery system comprising about 225 mg of gemcitabine into the bladder of the individual about once every three weeks (Q3W) for a total of up to six months following starting treatment, wherein each intravesical drug delivery system is removed from the bladder about three weeks after insertion into the bladder and wherein each intravesical drug delivery system is removed prior to insertion of another intravesical drug delivery system.

In some aspects, described herein are methods of treating Bacillus Calmette-Guérin (BCG)-naïve high-risk non-muscle invasive bladder cancer (HR-NMIBC) in an individual, comprising: intravenously administering to the individual about 360 mg of cetrelimab about once every three weeks (Q3W) for at least six months following starting treatment; and inserting an intravesical drug delivery system comprising about 256 mg of gemcitabine HCl (equivalent to about 225 mg gemcitabine free base) into the bladder of the individual about once every three weeks (Q3W) for a total of up to six months following starting treatment, wherein each intravesical drug delivery system is removed from the bladder about three weeks after insertion into the bladder and wherein each intravesical drug delivery system is removed prior to insertion of another intravesical drug delivery system.

In some aspects, described herein are methods of increasing event-free survival (EFS) for Bacillus Calmette-Guérin (BCG)-naïve high-risk non-muscle invasive bladder cancer (HR-NMIBC) in an individual, comprising: intravenously administering to the individual about 360 mg of cetrelimab about once every three weeks (Q3W) for at least six months following starting treatment; and inserting an intravesical drug delivery system comprising about 225 mg of gemcitabine into the bladder of the individual about once every three weeks (Q3W) for a total of up to six months following starting treatment, wherein each intravesical drug delivery system is removed from the bladder about three weeks after insertion into the bladder and wherein each intravesical drug delivery system is removed prior to insertion of another intravesical drug delivery system, and wherein the methods result in an increased event-free survival (EFS) of the individual compared to the EFS achieved by intravesical BCG, and/or wherein the methods result in an increased EFS in a population of patients receiving such therapy compared to a population of patients receiving intravesical BCG.

In some aspects, described herein are methods of increasing event-free survival (EFS) for Bacillus Calmette-Guérin (BCG)-naïve high-risk non-muscle invasive bladder cancer (HR-NMIBC) in an individual, comprising: intravenously administering to the individual about 360 mg of cetrelimab about once every three weeks (Q3W) for at least six months following starting treatment; and inserting an intravesical drug delivery system comprising about 256 mg of gemcitabine HCL (equivalent to about 225 mg gemcitabine free base) into the bladder of the individual about once every three weeks (Q3W) for a total of up to six months following starting treatment, wherein each intravesical drug delivery system is removed from the bladder about three weeks after insertion into the bladder and wherein each intravesical drug delivery system is removed prior to insertion of another intravesical drug delivery system, and wherein the methods result in an increased event-free survival (EFS) of the individual compared to the EFS achieved by intravesical BCG, and/or wherein the methods result in an increased EFS in a population of patients receiving such therapy compared to a population of patients receiving intravesical BCG.

In some aspects, described herein are methods of treating Bacillus Calmette-Guérin (BCG)-naïve high-risk non-muscle invasive bladder cancer (HR-NMIBC) in an individual, comprising: intravenously administering to the individual about 360 mg of cetrelimab about once every three weeks (Q3W) for at least six months following starting treatment; and inserting an intravesical drug delivery system comprising about 225 mg of gemcitabine into the bladder of the individual about once every three weeks (Q3W) for a total of up to six months following starting treatment, followed by inserting an intravesical drug delivery system comprising about 225 mg of gemcitabine into the bladder of the individual about once every 12 weeks (Q12W) for up to an additional 18 months after the first six months of treatment, wherein each intravesical drug delivery system is removed from the bladder about three weeks after insertion into the bladder and wherein each intravesical drug delivery system is removed prior to insertion of another intravesical drug delivery system.

In some aspects, described herein are methods of treating Bacillus Calmette-Guérin (BCG)-naïve high-risk non-muscle invasive bladder cancer (HR-NMIBC) in an individual, comprising: intravenously administering to the individual about 360 mg of cetrelimab about once every three weeks (Q3W) for at least six months following starting treatment; and inserting an intravesical drug delivery system comprising about 256 mg of gemcitabine HCl (equivalent to about 225 mg gemcitabine free base) into the bladder of the individual about once every three weeks (Q3W) for a total of up to six months following starting treatment, followed by inserting an intravesical drug delivery system comprising about 256 mg of gemcitabine HCl (equivalent to about 225 mg gemcitabine free base) into the bladder of the individual about once every 12 weeks (Q12W) for up to an additional 18 months after the first six months of treatment, wherein each intravesical drug delivery system is removed from the bladder about three weeks after insertion into the bladder and wherein each intravesical drug delivery system is removed prior to insertion of another intravesical drug delivery system.

In some aspects, described herein is a method of treating Bacillus Calmette-Guérin (BCG)-naïve high-risk non-muscle invasive bladder cancer (HR-NMIBC) in an individual, comprising inserting an intravesical drug delivery system comprising about 225 mg of gemcitabine into the bladder once every 3 weeks up to 6 months (8 doses), followed by once every 12 weeks for up to 2 years from first dose, or until persistent or recurrent HR-NMIBC, disease progression, or unacceptable toxicity. In some embodiments, the intravesical drug delivery system is administered as a monotherapy for treating Bacillus Calmette-Guérin (BCG)-naïve high-risk non-muscle invasive bladder cancer (HR-NMIBC).

In some aspects, described herein is a method of treating Bacillus Calmette-Guérin (BCG)-naïve high-risk non-muscle invasive bladder cancer (HR-NMIBC) in an individual, comprising: inserting an intravesical drug delivery system comprising about 225 mg of gemcitabine into the bladder once every 3 weeks up to 6 months (8 doses), followed by once every 12 weeks for up to 2 years from first dose, or until persistent or recurrent HR-NMIBC, disease progression, or unacceptable toxicity, and administering cetrelimab for up to 1 year from first dose, or until persistent or recurrent HR-NMIBC, disease progression, or unacceptable toxicity.

In some aspects, described herein is a method of treating Bacillus Calmette-Guérin (BCG)-naïve non-muscle invasive bladder cancer (NMIBC) in an individual, comprising inserting an intravesical drug delivery system comprising about 225 mg of gemcitabine into the bladder once every 3 weeks up to 6 months (8 doses), followed by once every 12 weeks for up to 2 years from first dose, or until persistent or recurrent NMIBC, disease progression, or unacceptable toxicity. In some embodiments, the intravesical drug delivery system is administered as a monotherapy for treating Bacillus Calmette-Guérin (BCG)-naïve non-muscle invasive bladder cancer (NMIBC).

In some aspects, described herein is a method of treating Bacillus Calmette-Guérin (BCG)-naïve non-muscle invasive bladder cancer (NMIBC) in an individual, comprising: inserting an intravesical drug delivery system comprising about 225 mg of gemcitabine into the bladder once every 3 weeks up to 6 months (8 doses), followed by once every 12 weeks for up to 2 years from first dose, or until persistent or recurrent NMIBC, disease progression, or unacceptable toxicity, and administering cetrelimab for up to 1 year from first dose, or until persistent or recurrent NMIBC, disease progression, or unacceptable toxicity. In some embodiments, each intravesical system is removed after a 3-week indwelling period.

In some aspects, described herein is a method of treating Bacillus Calmette-Guérin (BCG)-naïve non-muscle invasive bladder cancer (NMIBC) in an individual, comprising: inserting an intravesical drug delivery system comprising about 225 mg of gemcitabine into the bladder of the individual about once every three weeks (Q3W) for a total of up to six months following starting treatment, wherein each intravesical drug delivery system is removed from the bladder about three weeks after insertion into the bladder and wherein each intravesical drug delivery system is removed prior to insertion of another intravesical drug delivery system.

In some aspects, described herein is a method of increasing event-free survival (EFS) for Bacillus Calmette-Guérin (BCG)-naïve non-muscle invasive bladder cancer (NMIBC) in an individual, comprising: inserting an intravesical drug delivery system comprising about 225 mg of gemcitabine into the bladder of the individual about once every three weeks (Q3W) for a total of up to six months following starting treatment, wherein each intravesical drug delivery system is removed from the bladder about three weeks after insertion into the bladder and wherein each intravesical drug delivery system is removed prior to insertion of another intravesical drug delivery system, and wherein the method results in an increased event-free survival (EFS) of the individual compared to the EFS achieved by intravesical BCG, and/or wherein the method results in an increased EFS in a population of patients receiving such therapy compared to a population of patients receiving intravesical BCG.

In some embodiments, the method further comprises inserting an intravesical drug delivery system comprising about 225 mg of gemcitabine into the bladder of the individual about once every 12 weeks (Q12W) for up to an additional 18 months after the first six months of treatment.

In some aspects, described herein is a method of treating Bacillus Calmette-Guérin (BCG)-naïve non-muscle invasive bladder cancer (NMIBC) in an individual, comprising: inserting an intravesical drug delivery system comprising about 225 mg of gemcitabine into the bladder of the individual about once every three weeks (Q3W) for a total of up to six months following starting treatment, followed by inserting an intravesical drug delivery system comprising about 225 mg of gemcitabine into the bladder of the individual about once every 12 weeks (Q12W) for up to an additional 18 months after the first six months of treatment, wherein each intravesical drug delivery system is removed from the bladder about three weeks after insertion into the bladder and wherein each intravesical drug delivery system is removed prior to insertion of another intravesical drug delivery system.

In some embodiments, inserting the intravesical drug delivery system into the bladder of the individual about once every three weeks (Q3W) for a total of up to six months following starting treatment comprises administering to the bladder of the individual a plurality of intravesical drug delivery systems, each intravesical drug delivery system comprising about 225 mg of gemcitabine, wherein administering the plurality of intravesical drug delivery systems comprises two or more three-week dosing cycles of gemcitabine, comprising: (i) inserting one of the plurality of intravesical drug delivery systems into the bladder about once every three weeks (Q3W); and (ii) removing the intravesical drug delivery system from the bladder about three weeks after insertion, wherein in each dosing cycle of gemcitabine steps (i) and (ii) are repeated with a new one of the plurality of intravesical drug delivery systems for a total of up to six months following starting treatment.

In some embodiments, inserting the intravesical drug delivery system into the bladder of the individual about once every 12 weeks (Q12W) for up to an additional 18 months after the first six months of treatment comprises administering to the bladder of the individual a plurality of intravesical drug delivery systems, each intravesical drug delivery system comprising about 225 mg of gemcitabine, wherein administering the plurality of intravesical drug delivery systems comprises twelve-week dosing cycles of gemcitabine comprising: (iii) inserting a new one of the plurality of intravesical drug delivery systems into the bladder about once every twelve weeks (Q12W); and (iv) removing the intravesical drug delivery system from the bladder about three weeks after insertion, wherein in each twelve-week dosing cycle of gemcitabine steps (iii) and (iv) are repeated with a new one of the plurality of intravesical drug delivery systems for up to the additional eighteen months after the first six months of treatment.

In some aspects, described herein is a method of treating Bacillus Calmette-Guérin (BCG)-naïve non-muscle invasive bladder cancer (NMIBC) in an individual, comprising: intravenously administering to the individual about 360 mg of cetrelimab about once every three weeks (Q3W) for at least six months following starting treatment; and inserting an intravesical drug delivery system comprising about 225 mg of gemcitabine into the bladder of the individual about once every three weeks (Q3W) for a total of up to six months following starting treatment, wherein each intravesical drug delivery system is removed from the bladder about three weeks after insertion into the bladder and wherein each intravesical drug delivery system is removed prior to insertion of another intravesical drug delivery system.

In some aspects, described herein is a method of increasing event-free survival (EFS) for Bacillus Calmette-Guérin (BCG)-naïve non-muscle invasive bladder cancer (NMIBC) in an individual, comprising: intravenously administering to the individual about 360 mg of cetrelimab about once every three weeks (Q3W) for at least six months following starting treatment; and inserting an intravesical drug delivery system comprising about 225 mg of gemcitabine into the bladder of the individual about once every three weeks (Q3W) for a total of up to six months following starting treatment, wherein each intravesical drug delivery system is removed from the bladder about three weeks after insertion into the bladder and wherein each intravesical drug delivery system is removed prior to insertion of another intravesical drug delivery system, and wherein the method results in an increased event-free survival (EFS) of the individual compared to the EFS achieved by intravesical BCG, and/or wherein the method results in an increased EFS in a population of patients receiving such therapy compared to a population of patients receiving intravesical BCG.

In some embodiments, the method further comprises inserting an intravesical drug delivery system comprising about 225 mg of gemcitabine into the bladder of the individual about once every 12 weeks (Q12W) for up to an additional 18 months after the first six months of treatment.

In some aspects, described herein is a method of treating Bacillus Calmette-Guérin (BCG)-naïve non-muscle invasive bladder cancer (NMIBC) in an individual, comprising: intravenously administering to the individual about 360 mg of cetrelimab about once every three weeks (Q3W) for at least six months following starting treatment; and inserting an intravesical drug delivery system comprising about 225 mg of gemcitabine into the bladder of the individual about once every three weeks (Q3W) for a total of up to six months following starting treatment, followed by inserting an intravesical drug delivery system comprising about 225 mg of gemcitabine into the bladder of the individual about once every 12 weeks (Q12W) for up to an additional 18 months after the first six months of treatment, wherein each intravesical drug delivery system is removed from the bladder about three weeks after insertion into the bladder and wherein each intravesical drug delivery system is removed prior to insertion of another intravesical drug delivery system. In some embodiments, intravenously administering to the individual about 360 mg of cetrelimab about once every three weeks (Q3W) for at least six months comprises intravenously administering to the individual about 360 mg of cetrelimab about Q3W for about one year.

In some embodiments, inserting the intravesical drug delivery system into the bladder of the individual about once every three weeks (Q3W) for a total of up to six months following starting treatment comprises administering to the bladder of the individual a plurality of intravesical drug delivery systems, each intravesical drug delivery system comprising about 225 mg of gemcitabine, wherein administering the plurality of intravesical drug delivery systems comprises two or more three-week dosing cycles of gemcitabine, comprising: (i) inserting one of the plurality of intravesical drug delivery systems into the bladder about once every three weeks (Q3W); and (ii) removing the intravesical drug delivery system from the bladder about three weeks after insertion, wherein in each dosing cycle of gemcitabine steps (i) and (ii) are repeated with a new one of the plurality of intravesical drug delivery systems for a total of up to six months following starting treatment.

In some embodiments, inserting the intravesical drug delivery system into the bladder of the individual about once every 12 weeks (Q12W) for up to an additional 18 months after the first six months of treatment comprises administering to the bladder of the individual a plurality of intravesical drug delivery systems, each intravesical drug delivery system comprising about 225 mg of gemcitabine, wherein administering the plurality of intravesical drug delivery systems comprises twelve-week dosing cycles of gemcitabine comprising: (iii) inserting a new one of the plurality of intravesical drug delivery systems into the bladder about once every twelve weeks (Q12W); and (iv) removing the intravesical drug delivery system from the bladder about three weeks after insertion, wherein in each twelve-week dosing cycle of gemcitabine steps (iii) and (iv) are repeated with a new one of the plurality of intravesical drug delivery systems for up to the additional 18 months after the first six months of treatment.

In some embodiments, each intravesical drug delivery system comprises about 256 mg gemcitabine hydrochloride (HCl). In some embodiments, the administration of the cetrelimab and the insertion of the intravesical drug delivery system occurs no more than 72 hours apart from each other. In some embodiments, the administration of the cetrelimab and insertion of the intravesical drug delivery system occurs on the same day, and wherein the method comprises administering the intravesical drug delivery system at least 45 minutes prior to or after the completion of the administration of the cetrelimab.

In some embodiments, inserting another intravesical drug delivery system occurs within a week of removing a previous intravesical drug delivery system within the first six months of treatment. In some embodiments, after the first year of treatment, inserting another intravesical drug delivery system occurs within 7-11 weeks of removing a previous intravesical drug delivery system. In some embodiments, inserting another intravesical drug delivery system occurs on the same day as removing a previous intravesical drug delivery system within the first six months of treatment.

In some embodiments, inserting the intravesical drug delivery system into the bladder of the individual about once every 12 weeks (Q12W) for up to the additional 18 months begins at about week 36 after starting treatment, and wherein for a twelve-week period after the first six months of treatment, no intravesical drug delivery system is present in the bladder. In some embodiments, inserting the intravesical drug delivery system into the bladder of the individual about once every three weeks (Q3W) for a total of up to six months comprises eight three-week dosing cycles of gemcitabine.

In some embodiments, inserting the intravesical drug delivery system into the bladder of the individual about once every 12 weeks (Q12W) for up to an additional 18 months comprises up to six twelve-week dosing cycles of gemcitabine. In some embodiments, the method comprises up to fourteen total dosing cycles of gemcitabine. In some embodiments, intravenously administering the cetrelimab comprises eighteen dosing cycles of cetrelimab.

In some embodiments, the method further comprises, at about week 108 after start of treatment, inserting an intravesical drug delivery system comprising about 225 mg of gemcitabine into the bladder of the individual about once every 12 weeks (Q12W) for up to an additional ten months, wherein each intravesical drug delivery system is removed from the bladder about three weeks after insertion into the bladder and wherein each intravesical drug delivery system is removed prior to insertion of another intravesical drug delivery system.

In some embodiments, inserting an intravesical drug delivery system into the bladder of the individual about once every 12 weeks (Q12W) for up to an additional ten months comprises administering to the bladder of the individual a plurality of intravesical drug delivery systems, each intravesical drug delivery system comprising about 225 mg of gemcitabine, wherein administering the plurality of intravesical drug delivery systems comprises twelve-week dosing cycles of gemcitabine, comprising: (v) inserting a new one of the plurality of intravesical drug delivery systems into the bladder about once every twelve weeks (Q12W); and (vi) removing the intravesical drug delivery system from the bladder about three weeks after insertion, wherein in each twelve-week dosing cycle of gemcitabine steps (v) and (vi) are repeated with a new one of the plurality of intravesical drug delivery systems for up to the additional ten months.

In some embodiments, inserting the intravesical drug delivery system into the bladder of the individual about once every 12 weeks (Q12W) for up to the additional ten months begins at about week 108 after start of treatment. In some embodiments, inserting the intravesical drug delivery system into the bladder of the individual about once every 12 weeks (Q12W) for up to the additional ten months comprises up to four twelve-week dosing cycles of gemcitabine. In some embodiments, the method comprises up to eighteen total dosing cycles of gemcitabine.

In some embodiments, the method results in an increased event-free survival (EFS) compared to the EFS achieved by intravesical BCG, and/or wherein the method results in an increased EFS in a population of patients receiving such therapy compared to a population of patients receiving intravesical BCG. In some embodiments, an EFS event is defined by at least one of (a) positive local urine cytology, (b) positive local cystoscopy with biopsy proven high-risk disease, or (c) imaging proven disease progression or metastasis.

In some embodiments, the method comprises extending event-free survival (EFS) of the individual compared to the EFS achieved by intravesical BCG, and/or extending EFS in a population of patients receiving such therapy compared to a population of patients receiving intravesical BCG. In some embodiments, the method results in an increased overall complete response (CR) rate in a population of patients who have received treatment compared to the CR rate achieved by intravesical BCG. In some embodiments, the method results in an increased duration of CR compared to the duration of CR achieved by intravesical BCG. In some embodiments, the method results in an increased recurrence-free survival (RFS) compared to the RFS achieved by intravesical BCG. In some embodiments, the method results in an increased time to progression (TTP) compared to the TTP achieved by intravesical BCG. In some embodiments, the method results in an increased overall survival (OS) compared to the OS achieved by intravesical BCG. In some embodiments, the method results in an increased cancer specific survival (CSS) compared to the CSS achieved by intravesical BCG. In some embodiments, the method results in improved safety and tolerability compared to the safety and tolerability of intravesical BCG. In some embodiments, the method results in improved health-related quality of life (HRQOL) compared to the HRQOL of intravesical BCG.

In some embodiments, the individual has high-grade Ta stage bladder cancer. In some embodiments, the individual has T1 stage bladder cancer. In some embodiments, the individual has carcinoma in situ (CIS). In some embodiments, the individual has a mixed histology tumor with predominant urothelial differentiation. In some embodiments, the individual has an Eastern Cooperative Oncology Group (ECOG) performance status Grade 0, 1, or 2.

In some embodiments, the individual has papillary disease, and wherein the papillary disease is fully resected prior to treatment with gemcitabine. In some embodiments, the individual does not have a presence or history of histologically confirmed, muscle invasive, locally advanced, nonresectable, or metastatic urothelial carcinoma of stage T2 or greater. In some embodiments, the individual does not have urothelial carcinoma or a histological variant at any site outside of the bladder. In some embodiments, the individual does not have a history of NMIBC less than three years from current diagnosis. In some embodiments, the individual does not have visible papillary disease at the time of diagnosis. In some embodiments, the individual does not have N+ and/or M+ bladder cancer. In some embodiments, the individual does not have neuroendocrine, micropapillary, signet ring cell, plasmacytoid, or sarcomatoid features.

In some embodiments, the individual does not have a presence of any bladder or urothelial anatomical feature that prevents the safe insertion, indwelling use, and removal of the intravesical drug delivery system. In some embodiments, the individual does not have a history of clinically significant polyuria with recorded 24-hour urine volumes greater than 4000 mL. In some embodiments, the individual has not received BCG within three years from diagnosis. In some embodiments, the individual is instructed to drink at least 1500 mL of fluid per day during each dosing cycle.

In some embodiments, the method comprises administering anticholinergics, nonsteroidal anti-inflammatory drugs (NSAIDs), or bladder analgesics such as phenazopyridine prior to or after insertion or removal of the one of the plurality of intravesical drug delivery systems. In some embodiments, the method comprises administering prophylactic antibiotics with insertion and removal of the intravesical drug delivery system.

In some embodiments, the intravesical drug delivery system is inserted into the bladder transurethrally using a catheter. In some embodiments, the intravesical drug delivery system is removed using forceps and a cystoscope. In some embodiments, the intravesical drug delivery system comprises a flexible, bi-oval shaped housing comprising a dual lumen. In some embodiments, the dual lumen comprises a drug reservoir lumen containing the gemcitabine and a retention frame lumen containing a predefined shaped retention frame. In some embodiments, the retention frame is a super-elastic nitinol wire. In some embodiments, the drug reservoir lumen comprises a single delivery orifice that controllably releases the gemcitabine from the drug reservoir lumen through the delivery orifice. In some embodiments, the delivery orifice is a laser-drilled delivery orifice.

In some embodiments, the intravesical drug delivery system comprises a first unit contained within the drug reservoir lumen and comprising the gemcitabine and a second unit contained within the drug reservoir lumen in a position distinct from the first unit, the second unit comprising an osmotic agent. In some embodiments, the osmotic agent comprises urea minitablets. In some embodiments, the first unit further comprises urea. In some embodiments, the first unit contains at least 75 percent by weight gemcitabine HCL. In some embodiments, the second unit contains at least 85 percent by weight urea.

In some embodiments, the housing is configured to release the gemcitabine from the drug reservoir lumen through the delivery orifice via osmotic pressure generated by the osmotic agent. In some embodiments, the housing is elastically deformable between a retention shape configured to retain the intravesical drug delivery system in the individual's bladder and a deployment shape configured for passage of the intravesical drug delivery system through the individual's urethra. In some embodiments, the gemcitabine is in a non-liquid form. In some embodiments, the gemcitabine is in the form of minitablets.

In some embodiments, the intravesical drug delivery system releases gemcitabine at a zero-order release rate for an extended period, followed by a reduced, non-zero-order release rate over a decay period. In some embodiments, the concentration of gemcitabine in the urine of the individual is between about 4 μg/mL and about 50 μg/mL while the intravesical drug delivery system is inserted into the bladder.

In some embodiments, about 65% to about 85% of the gemcitabine in the intravesical drug delivery system is released into the urine of the individual by day seven after insertion into the bladder. In some embodiments, about 85% to about 100% of the gemcitabine in the intravesical drug delivery system is released into the urine of the individual by day 21 after insertion into the bladder.

In some embodiments, inserting the intravesical drug delivery system into the bladder of the individual about once every three weeks (Q3W) for a total of up to six months following starting treatment comprises the following steps: (i) inserting a first intravesical drug delivery system of the plurality of intravesical drug delivery systems into the bladder at about week 0; (ii) removing the first intravesical drug delivery system at about week 3; (iii) inserting a second intravesical drug delivery system of the plurality of intravesical drug delivery systems into the bladder at about week 3; (iv) removing the second intravesical drug delivery system at about week 6; (v) inserting a third intravesical drug delivery system of the plurality of intravesical drug delivery systems into the bladder at about week 6; (vi) removing the third intravesical drug delivery system at about week 9; (vii) inserting a fourth intravesical drug delivery system of the plurality of intravesical drug delivery systems into the bladder at about week 9; (viii) removing the fourth intravesical drug delivery system at about week 12; (ix) inserting a fifth intravesical drug delivery system of the plurality of intravesical drug delivery systems into the bladder at about week 12; (x) removing the fifth intravesical drug delivery system at about week 15; (xi) inserting a sixth intravesical drug delivery system of the plurality of intravesical drug delivery systems into the bladder at about week 15; (xii) removing the sixth intravesical drug delivery system at about week 18; (xiii) inserting a seventh intravesical drug delivery system of the plurality of intravesical drug delivery systems into the bladder at about week 18; (xiv) removing the seventh intravesical drug delivery system at about week 21; (xv) inserting an eighth intravesical drug delivery system of the plurality of intravesical drug delivery systems into the bladder at about week 21; and (xvi) removing the eighth intravesical drug delivery system at about week 24, wherein each intravesical drug delivery system comprises about 225 mg of gemcitabine.

In some embodiments, inserting the intravesical drug delivery system into the bladder of the individual about once every 12 weeks (Q12W) for up to the additional 18 months after the first six months of treatment comprises the following steps: (xvii) inserting a ninth intravesical drug delivery system of the plurality of intravesical drug delivery systems into the bladder at about week 36; (xviii) removing the ninth intravesical drug delivery system at about week 39; (xix) inserting a tenth intravesical drug delivery system of the plurality of intravesical drug delivery systems into the bladder at about week 48; (xx) removing the tenth intravesical drug delivery system at about week 51; (xxi) inserting an eleventh intravesical drug delivery system of the plurality of intravesical drug delivery systems into the bladder at about week 60; (xxii) removing the eleventh intravesical drug delivery system at about week 63; (xxiii) inserting a twelfth intravesical drug delivery system of the plurality of intravesical drug delivery systems into the bladder at about week 72; (xxiv) removing the twelfth intravesical drug delivery system at about week 75; (xxv) inserting a thirteenth intravesical drug delivery system of the plurality of intravesical drug delivery systems into the bladder at about week 84; (xxvi) removing the thirteenth intravesical drug delivery system at about week 87; (xxvii) inserting a fourteenth intravesical drug delivery system of the plurality of intravesical drug delivery systems into the bladder at about week 96; and (xxviii) removing the fourteenth intravesical drug delivery system at about week 99, wherein each intravesical drug delivery system comprises about 225 mg of gemcitabine.

In some embodiments, the total of up to six months ends with removal of an intravesical drug delivery system at about six months. In some embodiments, the additional 18 months after the first six months of treatment ends with removal of an intravesical drug delivery system at about 18 months following starting treatment. In some embodiments, each intravesical drug delivery system comprises about 256 mg of gemcitabine HCL.

### BRIEF DESCRIPTION OF THE DRAWINGS

The present application can be best understood by reference to the following description taken in conjunction with the accompanying figures included in the specification.

FIGS. 1A-1D show an exemplary intravesical drug delivery system, TAR-200, in accordance with some aspects. FIG. 1A illustrates the intravesical drug delivery system in a bi-oval retention shape, in accordance with some aspects. FIG. 1B illustrates an enlarged view of the silicone spacers and silicone adhesive at the ends of the housing of the intravesical drug delivery system of FIG. 1A, in accordance with some aspects. FIG. 1C illustrates a cross-sectional view of the housing of the intravesical drug delivery system along line C-C in FIG. 1A, in accordance with some aspects. FIG. 1D is a schematic depicting the intravesical drug delivery system of FIGS. 1A-1C in its elongated, uncoiled form, in accordance with some aspects.

FIG. 2A shows the percentage of gemcitabine released from the intravesical drug delivery system (TAR-200) over three weeks in vitro. Each line represents data from a single intravesical drug delivery system. FIG. 2B shows the percentage of gemcitabine released from the intravesical drug delivery system (TAR-200) over three weeks in vitro. The line represents an average from twelve intravesical drug delivery systems.

FIGS. 3A-3B show average urine concentration of gemcitabine in minipigs following transurethral intravesical deployment of TAR-200 for 7 days (FIG. 3A) and 28 days (FIG. 3B), respectively.

FIGS. 4A-4B show mean (SD) urine concentration of gemcitabine after intravesical administration of TAR-200 in combination with 360 mg of IV administered cetrelimab (Group 1), or TAR-200 alone (Group 2). FIG. 4A shows results from a first gemcitabine dosing cycle of three weeks. FIG. 4B shows results from a second, third, or fourth three-week dosing cycle (individuals provided samples for one of these dosing cycles).

FIGS. 5A-5B show mean (SD) Gemcitabine Related Components (GRC, Gemcitabine+dFdU) in mg-equivalents gemcitabine after intravesical administration of TAR-200 in combination with 360 mg of IV administered cetrelimab (Group 1), or TAR-200 alone (Group 2). FIG. 5A shows results from the first gemcitabine dosing cycle. FIG. 5B shows results from the second, third, or fourth dosing cycles (individuals provided samples for one of these dosing cycles).

FIG. 6 shows the amount of gemcitabine that is released from the intravesical drug delivery system in vitro vs. in vivo, as measured in human urine each day for seven days. The percentage reported is the percentage of the initial gemcitabine contained in the TAR-200 drug delivery system (225 mg) that was released each day.

FIGS. 7A-7B show the study design for a multi-center, open-label, randomized study evaluating the efficacy and safety of intravesical TAR-200 in combination with IV cetrelimab or TAR-200 alone versus intravesical BCG in participants with BCG-naïve HR-NMIBC. Asterisks refer to footnotes within figure. FIG. 7A shows the study design for study year 1. FIG. 7B shows the study design for study year 2 through optional study year 3.

FIGS. 8A-8B show an exemplary intravesical drug delivery system, TAR-200. TAR-200 is an intravesical drug delivery system comprising two components: (i) the drug constituent, which consists of gemcitabine minitablets (comprising 256 mg gemcitabine hydrochloride (equivalent to 225 mg of gemcitabine free base) and osmotic minitablets containing urea as the osmotic agent; and (ii) the intravesical device constituent, which is comprised of a dual lumen silicone part with a single laser-machined orifice and a superelastic nitinol wire (“wireform”). The large lumen of the silicone part contains the gemcitabine and urea minitablets and serves as an elementary osmotic pump to release drug in a controlled manner. The smaller lumen contains the nitinol wire in a predefined form to provide retention of the system in the bladder during the indwelling period. In FIG. 8A, TAR-200 is shown in an exemplary retention shape (e.g., in a so-called “pretzel shape” or a bi-oval shape) suited to retain the intravesical system within the bladder. The retention shape provides, among other things, that the intravesical drug delivery system resists becoming entrained in urine and excreted when the individual voids. In FIG. 8B, a close-up view is shown to highlight the drug constituent and wireform of TAR-200.

FIG. 9 shows the size of the intravesical drug delivery system, TAR-200, compared to a US 25-cent coin and 2-Euro coin.

FIGS. 10A-10B show exemplary insertion devices for use with intravesical drug delivery systems provided herein, such as TAR-200, called a urological placement catheter (Urinary Placement Catheter or Inserter) for the transurethral placement of the intravesical drug delivery systems. The UPC comprises a catheter and stylet, as shown in FIG. 10A. In FIG. 10B, a close-up view is shown to highlight the depth markings present on the catheter shaft (also referred to as a urinary catheter).

FIGS. 11A-11F are pictures of an exemplary sequence of loading TAR-200 into the Urinary Placement Catheter and deploying it therefrom. In FIG. 11A, TAR-200 is depicted next to the Urinary Placement Catheter and in the retention shape, suited to retain the device within the bladder. FIG. 11B shows a healthcare provider inserting TAR-200 into the catheter shaft of the Urinary Placement Catheter. FIG. 11C shows the healthcare provider pushing TAR-200 through the catheter shaft with the stylet of the Urinary Placement Catheter. FIG. 11D shows TAR-200 beginning to emerge from an exit port of the catheter shaft as the stylet is further pushed by the healthcare provider. FIG. 11E shows TAR-200 as it continues to exit from the exit port of the catheter shaft as the stylet is even further pushed by the healthcare provider.

FIG. 11F shows TAR-200 just prior to complete deployment from the catheter shaft, where it has substantially resumed the retention shape.

FIGS. 12A-12E show the development of a population PK (popPK) model from the study shown in FIGS. 7A-7B. FIG. 12A shows the model structure. FIG. 12B shows a summary of the parameter estimates of the final popPK model. FIG. 12C shows the visual predictive check (VPC) of the model. Solid line 1 represents the median, and dashed lines 2 represent 5th and 95th percentiles of the observed values. Solid line 3 represents the median, and dashed lines 4 represent 5th and 95th percentiles of the simulated values based on 1,000 simulations. Shaded area B represents the 95% confidence intervals of the median. Shaded areas A represents the 5th and 95th percentiles of the simulated values. Circles represent the observed values. FIG. 12D shows a simulated typical profile of cumulative release. FIG. 12E shows observed daily release.

### DETAILED DESCRIPTION

Described herein are methods of treating Bacillus Calmette-Guérin (BCG)-naïve high-risk non-muscle invasive bladder cancer (HR-NMIBC) in individuals by administering intravesical gemcitabine to the bladder. Also described herein are methods of treating Bacillus Calmette-Guérin (BCG)-naïve non-muscle invasive bladder cancer (NMIBC) in individuals by administering intravesical gemcitabine to the bladder. In some aspects, the methods for treating BCG-naïve HR-NMIBC and/or NMIBC further include administering (e.g., intravenously or subcutaneously) cetrelimab in combination with the intravesical gemcitabine. The gemcitabine can be administered by an intravesical drug delivery system that comprises about 225 mg gemcitabine and provides a controlled release of the gemcitabine into bladder urine during an indwelling period in the bladder.

In some embodiments, about 225 mg of gemcitabine is administered to the individual as a free base. In some embodiments, the about 225 mg of gemcitabine is administered to the individual as a free base equivalent (FBE). In some embodiments, the gemcitabine FBE is a pharmaceutically acceptable salt of gemcitabine. In some embodiments, the gemcitabine FBE is gemcitabine hydrochloride (HCl). In some embodiments, the about 225 mg gemcitabine free base is in the form of about 256 mg gemcitabine HCL. In some embodiments, this may be referred to as a 225 mg strength gemcitabine intravesical system.

As described herein, tolerability to BCG therapy remains an ongoing issue due to local and systemic toxicity and additional issues exist with BCG as a treatment option due to world-wide BCG supply concerns. The methods of treatment described herein can solve these and other problems by offering an alternative therapy, gemcitabine administered via an intravesical drug delivery system (alone or in combination with intravenous cetrelimab), to treat HR-NMIBC and/or NMIBC in BCG-naïve individuals.

### Definitions

Reference to “about” a value or parameter herein includes (and describes) variations that are directed to that value or parameter per se. For example, description referring to “about X” includes description of “X”. In another example, the term “about X-Y” used herein has the same meaning as “about X to about Y.”

In some embodiments, the term “about,” when used in association with a measurement or to modify a parameter or a value or a range of values, refers to variations of that measurement, parameter, value, or range of values of +/−10%, +/−9%, +/−8%, +/−7%, +/−6%, +/−5%, +/−4%, +/−3%, +/−2%, or +/−1%. For example, in some embodiments, “about X” includes and describes X+/−10%, +/−9%, +/−8%, +/−7%, +/−6%, +/−5%, +/−4%, +/−3%, +/−2%, or +/−1% of X. In some embodiments, the term “about” refers to variations of +/−5%, +/−4%, +/−3%, +/−2%, or +/−1%. In some embodiments, the term “about” refers to variations of +/−2% or +/−1%. In some embodiments, the term “about” refers to variations of +/−2%. In some embodiments, the term “about” refers to variations of +/−1%.

Reference to “at least” a value or parameter herein includes (and describes) variations that are directed to that value or parameter per se. For example, description referring to “at least X” includes description of “X”.

“Administration” or “administering” as used herein refers to providing a therapeutic agent to an individual. Administration can be subcutaneous, intravenous, or intravesical, for example. In some examples, a particular amount of drug is administered to the individual, for example by inserting an intravesical drug delivery system comprising the drug into the bladder of the individual, however only a portion of the drug is delivered (or released). In other embodiments, a particular amount of drug is administered to the individual, and the entire amount administered is delivered (or released) from the intravesical drug delivery system.

“Continuous” or “continuously” as used herein refers to sustained or extended release of a therapeutic agent (such as gemcitabine) over an extended period of time. Continuous includes different release rates over a period of time. For example, a drug is continuously released over a period of seven days if the drug is released at a faster rate over the first three days and a slower rate over a period of the last four days.

“Delivery” as used herein refers to release of a drug. For example, a delivery period is a period during which an effective amount or clinically effective amount, of a drug, such as gemcitabine, is released from, for example, an intravesical drug delivery system.

“Indwelling period” as used herein refers to a period in which an intravesical drug delivery system is present in the bladder of an individual. For example, an intravesical drug delivery system that is removed from a bladder three weeks after insertion has a three-week indwelling period.

“Individual” as used herein refers to a human.

“Treatment” or “treating” as used herein is an approach for obtaining beneficial or desired results including clinical results. For purposes of this invention, beneficial or desired clinical results include, but are not limited to, one or more of the following: alleviating one or more symptoms resulting from the disease, diminishing the extent of the disease, stabilizing the disease (e.g., preventing or delaying the worsening of the disease), suppressing or delaying the spread (e.g., metastasis) of the disease, preventing or delaying the recurrence of the disease, delaying or slowing the progression of the disease, ameliorating the disease state, providing a remission (partial or total) of the disease, decreasing the dose of one or more other medications required to treat the disease, delaying the progression of the disease, increasing or improving the quality of life, increasing weight gain, and/or prolonging survival. In some embodiments, “treatment” encompasses one or more clinical endpoints such as event-free survival, overall complete response rate, duration of complete response, recurrence-free survival, time to progression, overall survival, and/or cancer-specific survival.

“Methods of treatment” as used herein is intended to be synonymous with “medical use.” For example, disclosure of “a method of treating HR-NMIBC comprising administering gemcitabine to a patient” is equivalently to and includes “use of gemcitabine for treating HR-NMIBC in a patient.”

“Effective amount” used herein refers to an amount of a compound or composition sufficient to treat a specified disorder, condition or disease such as to ameliorate, palliate, lessen, and/or delay one or more of its symptoms. In reference to cancers, an effective amount comprises an amount sufficient to cause a tumor to shrink and/or to decrease the growth rate of the tumor (such as to suppress tumor growth) and/or to suppress, or delay other unwanted cell proliferation and/or to treat or suppress tumor metastasis and/or to reduce (such as eradiate) preexisting tumor metastasis and/or to reduce incidence or burden of preexisting tumor metastasis and/or to suppress or delay tumor metastasis and/or to inhibit tumor cells and/or to induce an immune response against a tumor cell. An effective amount can be administered in one or more administrations, for example, the effective amount of the drug or composition may: (i) reduce the number of cancer cells; (ii) reduce tumor size; (iii) inhibit, retard, slow to some extent and preferably stop cancer cell infiltration into peripheral organs; (iv) inhibit (i.e., slow to some extent and preferably stop) tumor metastasis; (v) inhibit tumor growth; (vi) suppress or delay occurrence and/or recurrence of tumor; and/or (vii) relieve to some extent one or more of the symptoms associated with the cancer.

“Monotherapy” as used herein means use of a treatment regimen, such as treatment comprising local administration of gemcitabine to the bladder, as the single active ingredient for treating HR-NMIBC and/or NMIBC. In some embodiments, the individual receiving monotherapy receives one or more therapy to treat a disease other than HR-NMIBC or a side effect of the monotherapy.

Gemcitabine can be administered to an individual as a gemcitabine free base equivalent, such as a pharmaceutically acceptable salt of gemcitabine (e.g., gemcitabine hydrochloride). For example, 256 mg of gemcitabine hydrochloride is equivalent to 225 mg gemcitabine free base. Amounts of gemcitabine refer to free base, unless indicated otherwise. Thus, as would be understood by the ordinarily-skilled artisan, unless indicated otherwise, the dosage amounts of gemcitabine as used herein refer to the dosage amount of gemcitabine free base. An individual may be administered a free base equivalent, such as a pharmaceutically acceptable salt thereof. It is understood that a corresponding free base equivalent of a pharmaceutically acceptable salt of gemcitabine may be readily determined. For a given amount of gemcitabine free base, the free base equivalent of a pharmaceutically acceptable salt may be determined as shown below:

$$“ X ” mg gem ( \frac{1 g gem}{1000 mg gem} ) ( \frac{1 mol gem}{263.2 g gem} ) ( \frac{1 mol gem FBE}{1 mol gem} ) ( \frac{“ Y ” g gem FBE}{1 mol gem FBE} ) ( \frac{1000 mg gem FBE}{1 g gem FBE} ) = mg gem FBE$$

To illustrate, the amount of gemcitabine HCL that corresponds to about 225 mg of gemcitabine free base may be calculated as follows

$$225 mg gem ( \frac{1 g gem}{1000 mg gem} ) ( \frac{1 mol gem}{263.2 g gem} ) ( \frac{1 mol gem FBE}{1 mol gem} ) ( \frac{299.66 g gem FBE}{1 mol gem FBE} ) ( \frac{1000 mg gem FBE}{1 g gem FBE} ) = 256.17 mg gem FBE$$

Thus, about 256 mg gemcitabine hydrochloride (also referred to as gemcitabine HCl; CAS Registry No. 122111-03-9) corresponds to, and is the free base equivalent of, about 225 mg gemcitabine free base (CAS Registry No. 95058-81-4, having the chemical structure

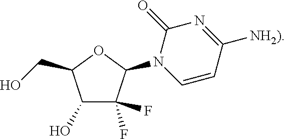

Similar calculations may be done for other salts or forms of gemcitabine to determine the amount of gemcitabine salt that corresponds to the free base dosage amount.

As used herein, the singular forms “a,” “an,” and “the” are intended to include the plural forms as well, unless the context clearly indicates otherwise. For example, “a” or “an” means “at least one” or “one or more.”

It is also to be understood that the term “and/or” as used herein refers to and encompasses any and all possible combinations of one or more of the associated listed items.

It is further to be understood that the terms “includes, “including,” “comprises,” and/or “comprising,” when used herein, specify the presence of stated features, integers, steps, operations, elements, components, and/or units but do not preclude the presence or addition of one or more other features, integers, steps, operations, elements, components, units, and/or groups thereof.

### Endpoint Definitions

“Event-free survival (EFS)” as used herein is defined as the time from randomization to either the time of the first recurrence of high-risk disease, progression, or death due to any cause, whichever occurs first. For participants with carcinoma in-situ (CIS), persistent disease at 6 months (Week 24) is also considered an EFS event. Progression is defined as: an increase of stage from Ta to T1 or from CIS to T1 or progression to muscle invasive bladder cancer (MIBC) (T greater than or equal to [>=]2) or to lymph node (N+) or to distant disease (M+), whichever occurs first.

“Overall Complete Response (CR) Rate” as used herein is the percentage of participants with CIS who have no presence of high-risk disease at 6 months.

“Duration of CR” as used herein is defined from the time of first CR achieved to first evidence of recurrence, progression or death due to any cause (whichever occurs first) for participants who achieve a CR.

“Recurrence-free survival (RFS)” as used herein is defined as the time from randomization to the time of the first recurrence of high-risk disease, or death due to any cause, whichever occurs first.

“Time to progression (TTP)” as used herein is defined as the time from randomization to the date of first documented evidence of disease progression or death due to disease progression, whichever occurs first.

“Overall survival (OS)” as used herein is defined as the time from randomization to death, due to any cause.

“Cancer specific survival (CSS)” as used herein is defined as the time from randomization to the date of death due to bladder cancer.

“Time to cystectomy (TTC)” as used herein is defined as the time from randomization to the date of cystectomy.

“Metastasis-free survival (MFS)” as used herein is defined as the time from randomization to first radiologic or histologic evidence of metastatic disease or death due to any cause.

“Event-free survival 2 (EFS2)” as used herein is defined as the time from randomization until the date of high-risk recurrence, progression, or death due to any cause on the first subsequent non-surgical anticancer treatment, whichever occurs first.

“All-cause event-free survival (aEFS)” as used herein includes EFS events and recurrence of non-high-risk NMIBC, and is defined as the time of randomization to either the time of EFS or any recurrence of non-high-risk NMIBC, whichever comes first.

### Methods of Treatment

Methods of treating Bacillus Calmette-Guérin (BCG)-naïve high-risk non-muscle invasive bladder cancer (HR-NMIBC) in an individual are provided herein. Also provided herein are methods of treating Bacillus Calmette-Guérin (BCG)-naïve non-muscle invasive bladder cancer (NMIBC) in an individual. The methods can include administering to the bladder of the individual an intravesical drug delivery system, comprising about 225 mg of gemcitabine. In some embodiments, gemcitabine in the intravesical drug delivery system is in the form of a pharmaceutically acceptable salt. In some embodiments, gemcitabine is in the form of gemcitabine HCL. In some embodiments, the intravesical drug delivery system comprises 256 mg of gemcitabine HCl (equivalent to about 225 mg gemcitabine free base) or another pharmaceutically acceptable salt. The intravesical drug delivery system can be administered alone (i.e., as a monotherapy) or in combination with intravenously administering cetrelimab to the individual (i.e., as a combination therapy). Accordingly, use of cetrelimab and an intravesical drug delivery system comprising gemcitabine for treating BCG-naïve HR-NMIBC in an individual are also provided herein. Further, use of cetrelimab and an intravesical drug delivery system comprising gemcitabine for treating BCG-naïve NMIBC in an individual are provided herein.

In some embodiments, the method comprises inserting an intravesical system comprising about 225 mg of gemcitabine into the bladder once every 3 weeks up to 6 months (8 doses), followed by once every 12 weeks for up to 2 years from first dose, or until persistent or recurrent HR-NMIBC, disease progression, or unacceptable toxicity. In some embodiments, the method comprises inserting an intravesical system comprising about 225 mg of gemcitabine into the bladder once every 3 weeks up to 6 months (8 doses), followed by once every 12 weeks for up to 2 years from first dose, or until persistent or recurrent NMIBC, disease progression, or unacceptable toxicity.

In some embodiments, the method comprises inserting an intravesical drug delivery system comprising about 225 mg of gemcitabine into the bladder once every 3 weeks up to 6 months (8 doses), followed by once every 12 weeks for up to 2 years from first dose, or until persistent or recurrent HR-NMIBC, disease progression, or unacceptable toxicity, and administering cetrelimab for up to 1 year from first dose, or until persistent or recurrent HR-NMIBC, disease progression, or unacceptable toxicity. In some embodiments, the method comprises inserting an intravesical drug delivery system comprising about 225 mg of gemcitabine into the bladder once every 3 weeks up to 6 months (8 doses), followed by once every 12 weeks for up to 2 years from first dose, or until persistent or recurrent NMIBC, disease progression, or unacceptable toxicity, and administering cetrelimab for up to 1 year from first dose, or until persistent or recurrent NMIBC, disease progression, or unacceptable toxicity. In some embodiments, each intravesical system is removed after a 3-week indwelling period. In some embodiments, the first dose refers to a first dose of gemcitabine. In some embodiments, the first dose refers to a first dose of cetrelimab.

As used herein, “dose” refers to one cycle of inserting and removing an intravesical device.

### Dosing Regimens

The treatment can include administering a plurality of intravesical drug delivery systems over about 2 years, the duration of which may be divided into a first phase and a second phase. The second phase may be followed by an additional optional third (e.g., follow-on maintenance) phase having a duration of about 1 year. The treatment methods can be a combination therapy that includes intravenously administering to the individual cetrelimab. In some embodiments, the first phase is an induction phase and the second phase is a maintenance phase. In some examples, the method of treatment is a monotherapy in which the intravesical drug delivery system is administered without intravenous cetrelimab. In some examples, as used herein “about three weeks” refers to a dosing window of three weeks±1 week in Year 1 of treatment, and a dosing window of three weeks±2 weeks in Year 2 and Year 3 of treatment.

### Treatment Phases

The treatment can include inserting an intravesical drug delivery system having about 225 mg of gemcitabine therein into the bladder of the individual about once every three weeks (Q3W) for up to a total of six months following starting treatment. The treatment can include inserting an intravesical drug delivery system having about 225 mg of gemcitabine therein into the bladder of the individual about once every three weeks (Q3W) for a total of about 24 weeks (i.e. ending with the removal of a device in week 24) following starting treatment. This period of up to a total of six months may otherwise be referred to herein as an induction phase or first phase. Each intravesical drug delivery system may be removed from the bladder about three weeks after insertion into the bladder and wherein each intravesical drug delivery system is removed prior to insertion of another intravesical drug delivery system. In some examples, inserting the intravesical drug delivery system into the bladder during the first phase includes administering to the bladder of the individual a plurality of intravesical drug delivery systems. In some embodiments, each intravesical drug delivery system comprises about 225 mg of gemcitabine. In some examples, the gemcitabine FBE is a pharmaceutically acceptable salt of gemcitabine. In some embodiments, the gemcitabine FBE is gemcitabine hydrochloride (HCl). In some embodiments, the intravesical drug delivery system comprises about 256 mg of gemcitabine HCl (equivalent to about 225 mg gemcitabine free base).

In some embodiments, the treatment comprises inserting an intravesical drug delivery system comprising about 225 mg of gemcitabine into the bladder once every 3 weeks up to 6 months (8 doses), followed by once every 12 weeks for up to 2 years from first dose, or until persistent or recurrent HR-NMIBC, disease progression, or unacceptable toxicity. In some embodiments, the treatment comprises inserting an intravesical drug delivery system comprising about 225 mg of gemcitabine into the bladder once every 3 weeks up to 6 months (8 doses), followed by once every 12 weeks for up to 2 years from first dose, or until persistent or recurrent NMIBC, disease progression, or unacceptable toxicity. In some embodiments, the method further comprises administering cetrelimab for up to 1 year from first dose, or until persistent or recurrent HR-NMIBC, disease progression, or unacceptable toxicity. In some embodiments, the first dose refers to a first dose of gemcitabine. In some embodiments, the first dose refers to a first dose of cetrelimab.

The plurality of intravesical drug delivery systems may be multiple copies of the same intravesical drug delivery system. Stated another way, each intravesical drug delivery system may have the same structure, the same amount of gemcitabine contained therein, etc.

Administering the plurality of intravesical drug delivery systems during the first phase can include two or more three-week dosing cycles of gemcitabine. Each three-week dosing cycle of gemcitabine may include the following steps: (i) inserting one of the plurality of intravesical drug delivery systems into the bladder about once every three weeks (Q3W); and (ii) removing the intravesical drug delivery system from the bladder about three weeks after insertion. Each of these steps (i) and (ii) can be repeated with a new one of the plurality of intravesical drug delivery systems for up to a total of six months, 24 weeks, or a total of 8 times. In some embodiments, the first phase starts in week 0 with insertion of an intravesical drug delivery system and ends with removal of an intravesical drug delivery system on about week 24.

Following the first (or induction) phase, the treatment methods can include a second (or maintenance) phase that includes inserting an intravesical drug delivery system comprising about 225 mg of gemcitabine into the bladder of the individual about once every 12 weeks (Q12W) for up to an additional 18 months after the first six months of treatment. The second phase may begin at week 24 and/or month six. Similar to the first phase, each intravesical drug delivery system may be removed from the bladder about three weeks after insertion into the bladder and wherein each intravesical drug delivery system is removed prior to insertion of another intravesical drug delivery system during the second phase. Inserting the intravesical drug delivery system during the second phase can include administering to the bladder of the individual a plurality of intravesical drug delivery systems. Similar to the first phase, each intravesical drug delivery system can comprise about 225 mg of gemcitabine or about 256 mg gemcitabine HCL. In some embodiments, the second phase begins on about week 24 and the first intravesical drug delivery device in the second phase is inserted into the bladder in about week 36. In some embodiments, there is a period of time between the start of the second phase and the insertion of the first intravesical drug delivery system of the second phase. In some embodiments, the second phase starts on about week 24 with removal of an intravesical drug delivery system and ends with the removal of an intravesical drug delivery system on about week 99.

Administering the plurality of intravesical drug delivery systems during the second phase can include twelve-week dosing cycles of gemcitabine. Each twelve-week dosing cycle of gemcitabine may include the following steps: (i) inserting a new one of the plurality of intravesical drug delivery systems into the bladder about once every twelve weeks (Q12W); and (ii) removing the intravesical drug delivery system from the bladder about three weeks after insertion. In each twelve-week dosing cycle of gemcitabine, steps (iii) and (iv) can be repeated with a new one of the plurality of intravesical drug delivery systems for up to eighteen months. In some examples, each twelve-week cycle includes a three-week period during which the intravesical drug delivery system is retained in the bladder of the individual and a nine-week period (e.g., rest period) wherein there is no intravesical drug delivery device in the bladder. In some examples, step i) of the cycle begins on week nine of the twelve-week dosing cycle. Stated another way, in some examples, the second phase begins on week 24 with a nine-week period wherein there is no intravesical drug delivery device in the bladder followed by insertion of an intravesical drug delivery system on about week 36. In some examples, the twelve-week dosing cycle includes a nine-week period, followed by (i) inserting a new one of the plurality of intravesical drug delivery systems into the bladder and (ii) removing the intravesical drug delivery system from the bladder about three weeks after insertion.

In some examples, in the first phase, inserting a new intravesical drug delivery system occurs within a week of removing a previous intravesical drug delivery system. For example, the new intravesical drug delivery system may be inserted on the same day or within 1, 2, 3, 4, 5, 6, or 7 days of removing the previous intravesical drug delivery system. In some examples, this ±1-week window may be applicable all years of the treatment.

In some examples, in the second phase, inserting the new intravesical drug delivery system occurs within about nine weeks of removing a previous intravesical drug delivery system. For example, the new intravesical drug delivery system may be inserted within 7-11 or within 8-10 weeks of removing the previous intravesical drug delivery system. In some examples, this ±2-week window surrounding the nine-week period may be applicable only after the first year of treatment.

In some examples, dosing during the second phase begins after a period (e.g., a rest period) of about twelve weeks following the first phase. No intravesical drug delivery system may be present in the bladder during this twelve-week period. This period may be considered as part of the second phase, although no intravesical drug delivery system is present in the bladder during this time.

In some examples, administering the plurality of intravesical drug delivery systems during the first phase includes a total of eight three-week dosing cycles of gemcitabine. Stated another way, eight intravesical drug delivery systems may be sequentially inserted into the bladder during the first phase, one for each three-week dosing cycle of gemcitabine. In some examples, administering the plurality of intravesical drug delivery systems during the second phase includes up to six twelve-week dosing cycles of gemcitabine. Stated another way, six intravesical drug delivery systems may be sequentially inserted into the bladder during the second phase, one for each twelve-week dosing cycle of gemcitabine. During the second phase, the new intravesical drug delivery system may be inserted about nine weeks after the previous intravesical drug delivery system is removed.

In some examples, the treatment (including both the first phase and the second phase) includes up to fourteen total dosing cycles of gemcitabine. Stated another way, fourteen intravesical drug delivery systems may be sequentially inserted into the bladder over the course of the treatment, one for each dosing cycle of gemcitabine.

In some examples, the method comprises inserting the intravesical drug delivery system into the bladder of the individual about once every three weeks (Q3W) for a total of up to six months following starting treatment comprising the following steps:
(i) inserting a first intravesical drug delivery system of the plurality of intravesical drug delivery systems into the bladder at about week 0;
(ii) removing the first intravesical drug delivery system at about week 3;
(iii) inserting a second intravesical drug delivery system of the plurality of intravesical drug delivery systems into the bladder at about week 3;
(iv) removing the second intravesical drug delivery system at about week 6;
(v) inserting a third intravesical drug delivery system of the plurality of intravesical drug delivery systems into the bladder at about week 6;
(vi) removing the third intravesical drug delivery system at about week 9;
(vii) inserting a fourth intravesical drug delivery system of the plurality of intravesical drug delivery systems into the bladder at about week 9;
(viii) removing the fourth intravesical drug delivery system at about week 12;
(ix) inserting a fifth intravesical drug delivery system of the plurality of intravesical drug delivery systems into the bladder at about week 12;
(x) removing the fifth intravesical drug delivery system at about week 15;
(xi) inserting a sixth intravesical drug delivery system of the plurality of intravesical drug delivery systems into the bladder at about week 15;
(xii) removing the sixth intravesical drug delivery system at about week 18;
(xiii) inserting a seventh intravesical drug delivery system of the plurality of intravesical drug delivery systems into the bladder at about week 18;
(xiv) removing the seventh intravesical drug delivery system at about week 21;
(xv) inserting an eighth intravesical drug delivery system of the plurality of intravesical drug delivery systems into the bladder at about week 21; and
(xvi) removing the eighth intravesical drug delivery system at about week 24, wherein each of the plurality of intravesical drug delivery systems comprises about 225 gemcitabine (or about 256 mg gemcitabine HCl).

In some examples, the method comprises inserting the intravesical drug delivery system into the bladder of the individual about once every 12 weeks (Q12W) for up to an additional 18 months after the first six months of treatment comprising the following steps:
(i) inserting a ninth intravesical drug delivery system of the plurality of intravesical drug delivery systems into the bladder at about week 36;
(ii) removing the ninth intravesical drug delivery system at about week 39;
(iii) inserting a tenth intravesical drug delivery system of the plurality of intravesical drug delivery systems into the bladder at about week 48;
(iv) removing the tenth intravesical drug delivery system at about week 51;
(v) inserting an eleventh intravesical drug delivery system of the plurality of intravesical drug delivery systems into the bladder at about week 60;
(vi) removing the eleventh intravesical drug delivery system at about week 63;
(vii) inserting a twelfth intravesical drug delivery system of the plurality of intravesical drug delivery systems into the bladder at about week 72;
(viii) removing the twelfth intravesical drug delivery system at about week 75;
(ix) inserting a thirteenth intravesical drug delivery system of the plurality of intravesical drug delivery systems into the bladder at about week 84;
(x) removing the thirteenth intravesical drug delivery system at about week 87;
(xi) inserting a fourteenth intravesical drug delivery system of the plurality of intravesical drug delivery systems into the bladder at about week 96; and
(xii) removing the fourteenth intravesical drug delivery system at about week 99, wherein each of the plurality of intravesical drug delivery systems comprises about 225 gemcitabine (or about 256 mg gemcitabine HCl).

In any of the above examples, the previous intravesical drug delivery system is removed before a subsequent intravesical drug delivery system is inserted such that there is no more than one intravesical drug delivery system in the bladder of the individual at any one time.

In some examples, the methods of treatment include disease assessments over the course of the therapy. For example, the methods of treatment can include at least one cystoscopy and/or urine cytology over the treatment. The methods of treatment may include a cystoscopy and/or cytology at about weeks 12, 24, 36, and/or 48 of treatment. In some examples, the methods of treatment include a urine cytology at about week 21 for biopsy/TURBT planning.

The methods of treatment may include a transurethral resection of bladder tumor (TURBT)/bladder biopsy over the course of the treatment. For example, the methods of treatment may include TURBT/bladder biopsy at about week 24 (i.e., at the end of the induction or first phase). The TURBT/bladder biopsy may be clinically indicated for participants with CIS (including those participants with negative cystoscopy results) based on local urine cytology. For participants with high-grade Ta/any T1 disease, the TURBT/bladder biopsy may be performed as clinically indicated based on local urine cytology and cystoscopy to determine the presence or absence of recurrent, or progressive disease and wherein each intravesical drug delivery system is removed prior to administration of dosing in the maintenance or second phase.

The methods of treatment may include at least one computed tomography (CT) urogram/magnetic resonance (MR) urogram over the course of the treatment. For example, the methods of treatment may include CT urogram/MR urogram at about week 24 and/or week 48.

### Cetrelimab Dosing

As noted above, the methods of treatment described herein may be a combination therapy that includes administering gemcitabine to the individual and administering cetrelimab (e.g., intravenously or subcutaneously) to the individual. In some examples, administering cetrelimab includes intravenously administering about 360 mg of cetrelimab about once every three weeks (Q3W) for about one year. In some examples, administering cetrelimab includes intravenously administering to the individual about 360 mg of cetrelimab about once every three weeks (Q3W) for at least six months. In some examples, administering cetrelimab includes subcutaneously administering the cetrelimab to the individual at a loading dose of about 900 mg at starting treatment, followed by about a 600 mg dose of cetrelimab about every three weeks (Q3W) for a total of up to six months. In some embodiments, the methods of treatment described herein may be a combination therapy that includes administering gemcitabine HCL to the individual and administering cetrelimab (e.g., intravenously) to the individual.

In some embodiments, the first administration of cetrelimab starts in week 0 of a dosing regimen. In some embodiments, cetrelimab is administered as a combination therapy with an intravesical drug delivery system comprising gemcitabine, wherein both cetrelimab and the intravesical drug delivery system are administered on the same three week cycle. In some embodiments, the cetrelimab is administered in the same week, or on the same day that the intravesical drug delivery system is inserted into the bladder. Cetrelimab may be administered before or after insertion of the drug delivery system. In some embodiments, cetrelimab is administered as a combination therapy with an intravesical drug delivery system comprising gemcitabine HCL.

In some examples, the cetrelimab for intravenous administration is diluted into a volume of between about 100 ml and 1000 ml prior to administration. In some examples, the duration of the intravenous administration is between about 20 minutes and about 80 minutes. In some examples, the duration of the intravenous administration is between about 50 minutes and about 70 minutes.

In some examples, intravenously administering the cetrelimab includes about eighteen dosing cycles of cetrelimab. The cetrelimab may be administered at each of about weeks 0, 3, 6, 9, 12, 15, 18, 21, 24, 27, 30, 33, 36, 39, 42, 45, 48, and 51.

In some examples, subcutaneously administering the cetrelimab includes about eight dosing cycles of cetrelimab. The cetrelimab may be administered at each of about weeks 0, 3, 6, 9, 12, 15, 18, 21, and 24.

In some embodiments, each dosing cycle of cetrelimab is about three weeks and starts with administration of cetrelimab. In some embodiments, each dosing cycle of cetrelimab is about three weeks and comprises a single administration of cetrelimab.

In some examples, intravenously administering the cetrelimab in combination with the plurality of intravesical drug delivery systems during the first phase comprises the following steps:
(i) intravenously administering about 360 mg cetrelimab and inserting a first intravesical drug delivery system of the plurality of intravesical drug delivery systems into the bladder at about week 0, simultaneously, or sequentially in any order;
(ii) removing the first intravesical drug delivery system at about week 3;
(iii) intravenously administering about 360 mg cetrelimab and inserting a second intravesical drug delivery system of the plurality of intravesical drug delivery systems into the bladder at about week 3 simultaneously, or sequentially in any order;
(iv) removing the second intravesical drug delivery system at about week 6;
(v) intravenously administering about 360 mg cetrelimab and inserting a third intravesical drug delivery system of the plurality of intravesical drug delivery systems into the bladder at about week 6 simultaneously, or sequentially in any order;
(vi) removing the third intravesical drug delivery system at about week 9;
(vii) intravenously administering about 360 mg cetrelimab and inserting a fourth intravesical drug delivery system of the plurality of intravesical drug delivery systems into the bladder at about week 9 simultaneously, or sequentially in any order;
(viii) removing the fourth intravesical drug delivery system at about week 12;
(ix) intravenously administering about 360 mg cetrelimab and inserting a fifth intravesical drug delivery system of the plurality of intravesical drug delivery systems into the bladder at about week 12 simultaneously, or sequentially in any order;
(x) removing the fifth intravesical drug delivery system at about week 15;
(xi) intravenously administering about 360 mg cetrelimab and inserting a sixth intravesical drug delivery system of the plurality of intravesical drug delivery systems into the bladder at about week 15 simultaneously, or sequentially in any order;
(xii) removing the sixth intravesical drug delivery system at about week 18;
(xiii) intravenously administering about 360 mg cetrelimab and inserting a seventh intravesical drug delivery system of the plurality of intravesical drug delivery systems into the bladder at about week 18 simultaneously, or sequentially in any order;
(xiv) removing the seventh intravesical drug delivery system at about week 21;
(xv) intravenously administering about 360 mg cetrelimab and inserting an eighth intravesical drug delivery system of the plurality of intravesical drug delivery systems into the bladder at about week 21 simultaneously, or sequentially in any order; and
(xvi) intravenously administering about 360 mg cetrelimab and removing the eighth intravesical drug delivery system at about week 24 simultaneously, or sequentially in any order, wherein each of the plurality of intravesical drug delivery systems comprises about 225 mg gemcitabine (or about 256 mg gemcitabine HCl).

In some examples, subcutaneously administering the cetrelimab in combination with the plurality of intravesical drug delivery systems during the first phase comprises the following steps:
i) inserting a first intravesical drug delivery system into the bladder and administering about 900 mg cetrelimab subcutaneously at about week 0 simultaneously, or sequentially in any order;
ii) removing the first intravesical drug delivery system at about week 3;
iii) inserting a second intravesical drug delivery system into the bladder and administering about 600 mg cetrelimab subcutaneously at about week 3 simultaneously, or sequentially in any order;
iv) removing the second intravesical drug delivery system at about week 6;
v) inserting a third intravesical drug delivery system into the bladder and administering about 600 mg cetrelimab subcutaneously at about week 6 simultaneously, or sequentially in any order;
vi) removing the third intravesical drug delivery system at about week 9;
vii) inserting a fourth intravesical drug delivery system into the bladder and administering about 600 mg cetrelimab subcutaneously at about week 9 simultaneously, or sequentially in any order;
viii) removing the fourth intravesical drug delivery system at about week 12;
ix) inserting a fifth intravesical drug delivery system into the bladder and administering about 600 mg cetrelimab subcutaneously at about week 12 simultaneously, or sequentially in any order;
x) removing the fifth intravesical drug delivery system at about week 15;
xi) inserting a sixth intravesical drug delivery system into the bladder and administering about 600 mg cetrelimab subcutaneously at about week 15 simultaneously, or sequentially in any order;
xii) removing the sixth intravesical drug delivery system at about week 18;
xiii) inserting a seventh intravesical drug delivery system into the bladder and administering about 600 mg cetrelimab subcutaneously at about week 18 simultaneously, or sequentially in any order;
xiv) removing the seventh intravesical drug delivery system at about week 21;
xv) inserting an eighth intravesical drug delivery system into the bladder and administering about 600 mg cetrelimab subcutaneously at about week 21 simultaneously, or sequentially in any order; and
xvi) intravenously administering about 360 mg cetrelimab and removing the eighth intravesical drug delivery system at about week 24 simultaneously, or sequentially in any order; wherein each of the plurality of intravesical drug delivery systems comprises about 225 mg gemcitabine (or about 256 mg gemcitabine HCl).

In some examples, administration of the cetrelimab and the insertion of the intravesical drug delivery system occurs no more than 72 hours apart from each other. For example, the cetrelimab may be administered and an intravesical drug delivery system may be inserted into the bladder within about 0-72 hours, 0-48 hours, 0-24 hours, or 0-12 hours apart from one another. In some examples, the administration of the cetrelimab and insertion of the intravesical drug delivery system occurs on the same day. The cetrelimab may be administered prior to inserting the intravesical drug delivery system. In some examples, the intravesical drug delivery system may be inserted prior to administering the cetrelimab.

In some examples, administering the intravesical drug delivery system occurs at least 45 minutes prior to or after the completion of the administration of the cetrelimab. For example, administering the intravesical drug delivery system may occur at least 45 minutes, 1 hour, 1.5 hours, 2 hours, 4 hours, 6 hours, 8 hours, 10 hours, or 12 hours prior to or after the completion of the administration of the cetrelimab.

Cetrelimab is a fully human immunoglobulin G4 (IgG4) kappa monoclonal antibody containing the hinge stabilizing S228P mutation. Cetrelimab binds to programmed cell death protein 1 (PD 1) with high affinity and specificity, blocks binding to the programmed-cell death ligands 1 and 2 (PD L1 and PD-L2), enhances pro-inflammatory cytokine production from ex-vivo stimulated T cells, and reduces tumor volume in human PD-1 knock-in mice bearing MC38 murine colon carcinoma tumors.

Cetrelimab is characterized by the following amino acid sequences: the CDRH1 of SEQ ID NO: 1, the CDRH2 of SEQ ID NO: 2, the CDRH3 of SEQ ID NO: 3, the CDRL1 of SEQ ID NO: 4, the CDRL2 of SEQ ID NO: 5, the CDRL3 of SEQ ID NO: 6, the VH of SEQ ID NO: 7, the VL of SEQ ID NO: 8, the heavy chain of SEQ ID NO: 9 and the light of SEQ ID NO: 10. The amino acid sequences of Cetrelimab are provided below in Table 1. Additional anti-PD1 antibodies are disclosed in U.S. Pat. No. 10,894,830, the contents of which are incorporated herein by reference in its entirety.

TABLE 1
Cetrelimab Amino Acid Sequences

| | | |
|---|---|---|
| HCDR1 | SEQ ID | SYAIS |
| | NO: 1 | |
| HCDR2 | SEQ ID | GIIPIFDTANYAQKFQG |
| | NO: 2 | |
| HCDR3 | SEQ ID | PGLAAAYDTGSLDY |
| | NO: 3 | |
| LCDR1 | SEQ ID | RASQSVRSYLA |
| | NO: 4 | |
| LCDR2 | SEQ ID | DASNRAT |
| | NO: 5 | |
| LCDR3 | SEQ ID | QQRNYWPLT |
| | NO: 6 | |
| heavy chain | SEQ ID | QVQLVQSGAEVKKPGSSVKVSCKASGGTFSSYAISWVRQAPGQGLEWMGG |
| variable | NO: 7 | IIPIFDTANYAQKFQGRVTITADESTSTAYMELSSLRSEDTAVYYCARPG |
| region (VH) | | LAAAYDTGSLDYWGQGTLVTVSS |
| light chain | SEQ ID | EIVLTQSPATLSLSPGERATLSCRASQSVRSYLAWYQQKPGQAPRLLIYD |
| variable | NO: 8 | ASNRATGIPARFSGSGSGTDFTLTISSLEPEDFAVYYCQQRNYWPLTFGQ |
| region (VL) | | GTKVEIK |
| heavy chain | SEQ ID | QVQLVQSGAEVKKPGSSVKVSCKASGGTFSSYAISWVRQAPGQGLEWMGG |
| | NO: 9 | IIPIFDTANYAQKFQGRVTITADESTSTAYMELSSLRSEDTAVYYCARPG |
| | | LAAAYDTGSLDYWGQGTLVTVSSASTKGPSVFPLAPCSRSTSESTAALGC |
| | | LVKDYFPEPVTVSWNSGALTSGVHTFPAVLQSSGLYSLSSVVTVPSSSLG |
| | | TKTYTCNVDHKPSNTKVDKRVESKYGPPCPPCPAPEFLGGPSVFLFPPKP |
| | | KDTLMISRTPEVTCVVVDVSQEDPEVQFNWYVDGVEVHNAKTKPREEQFN |
| | | STYRVVSVLTVLHQDWLNGKEYKCKVSNKGLPSSIEKTISKAKGQPREPQ |
| | | VYTLPPSQEEMTKNQVSLTCLVKGFYPSDIAVEWESNGQPENNYKTTPPV |
| | | LDSDGSFFLYSRLTVDKSRWQEGNVFSCSVMHEALHNHYTQKSLSLSLGK |
| light chain | SEQ ID | EIVLTQSPATLSLSPGERATLSCRASQSVRSYLAWYQQKPGQAPRLLIYD |
| | NO: 10 | ASNRATGIPARFSGSGSGTDFTLTISSLEPEDFAVYYCQQRNYWPLTFGQ |
| | | GTKVEIKRTVAAPSVFIFPPSDEQLKSGTASVVCLLNNFYPREAKVQWKV |
| | | DNALQSGNSQESVTEQDSKDSTYSLSSTLTLSKADYEKHKVYACEVTHQG |
| | | LSSPVTKSFNRGEC |

In some embodiments, the cetrelimab is administered or provided for administration in a pharmaceutical composition comprising between about 10 mg/ml to about 150 mg/ml (e.g., 30 mg/ml, 150 mg/ml, etc.) of the cetrelimab and one or more pharmaceutically acceptable excipients. Pharmaceutically acceptable excipients can refer to solvents, dispersion media, coatings, antibacterial and antifungal agents, isotonic and absorption delaying agents, and the like that are physiologically compatible, such as salts, buffers, antioxidants, saccharides, aqueous or non-aqueous carriers, preservatives, wetting agents, surfactants or emulsifying agents, or combinations thereof. In some examples, the cetrelimab includes proline at position 228, residue numbering according to the EU Index. In some examples, the cetrelimab has at least one substitution in an Fc region to modulate antibody effector functions or antibody half-life.

### Additional Phases

As noted above, the methods of treatment may include a third or additional phase. The third phase may begin in about year 3, in about month 25, and/or at about the 108 week of treatment. The third phase may include inserting an intravesical drug delivery system comprising about 225 mg of gemcitabine into the bladder of the individual about once every 12 weeks (Q12W) for up to an additional ten months. In some embodiments, the intravesical drug delivery system comprises about 256 mg of gemcitabine HCL. As described herein with respect to the first and second phases, each intravesical drug delivery system is removed from the bladder about three weeks after insertion into the bladder and wherein each intravesical drug delivery system is removed prior to insertion of another intravesical drug delivery system. In some examples, inserting the intravesical drug delivery system into the bladder during the third phase includes administering to the bladder of the individual a plurality of intravesical drug delivery systems in accordance with twelve-week dosing cycles of gemcitabine. Each twelve-week dosing cycle may include the following steps: (v) inserting a new one of the plurality of intravesical drug delivery systems into the bladder about once every twelve weeks (Q12W); and (vi) removing the intravesical drug delivery system from the bladder about three weeks after insertion. In each twelve-week dosing cycle of gemcitabine, steps (v) and (vi) can be repeated with a new one of the plurality of intravesical drug delivery systems for up to ten months. In some examples, each twelve-week cycle includes a three-week period during which the intravesical drug delivery system is retained in the bladder of the individual and a nine-week period wherein there is no intravesical drug delivery system in the bladder. In some examples, step i) of the cycle begins on week nine of the twelve-week dosing cycle. In some examples, the twelve-week dosing cycle includes a nine-week period during which no intravesical drug delivery system is present in the bladder, followed by (i) inserting a new one of the plurality of intravesical drug delivery systems into the bladder and (ii) removing the intravesical drug delivery system from the bladder about three weeks after insertion.

In some examples, dosing during the third phase begins after a period of about nine weeks following the second phase. As noted above, no intravesical drug delivery system is present in the bladder during this period. This period may be considered as part of the third or additional phase, although no intravesical drug delivery system is present in the bladder during this time. In some embodiments, dosing during the third phase begins on about week 108 or about month 25 of the dosing schedule.

In some examples, administering the plurality of intravesical drug delivery systems during the third phase includes up to four twelve-week dosing cycles of gemcitabine. Stated another way, up to four intravesical drug delivery systems may be sequentially inserted into the bladder during the third phase, one for each twelve-week dosing cycle of gemcitabine. In some examples, the third phase includes less than four twelve-week dosing cycles of gemcitabine, such as three, two, or one twelve-week dosing cycle of gemcitabine.

In some examples, the treatment (including each of the first phase, the second phase, and the third phase) includes up to eighteen total dosing cycles of gemcitabine. Stated another way, eighteen intravesical drug delivery systems may be sequentially inserted into the bladder over the course of the treatment, one for each dosing cycle of gemcitabine. In some embodiments, the second phase starts at about 6 months and ends at about 24 months following starting treatment.

In some examples, the gemcitabine is released from the intravesical drug delivery system into the urine in the bladder during a delivery period that is less than the indwelling period. For example, the indwelling period may be about three weeks, although the gemcitabine may be released from the intravesical drug delivery system over a delivery period of about one week, about two weeks, or any other time less than three weeks (e.g., between about one to three weeks). The intravesical drug delivery system may be configured to release gemcitabine for a delivery period of at least about one week (e.g., 7 days). In some embodiments, the method comprises a delivery period of about 7 days.

In some examples, the concentration of gemcitabine in the urine of the individual may be between about 4 μg/mL and about 50 μg/mL while the intravesical drug delivery system is inserted into the bladder. In some examples, the concentration of gemcitabine in the urine of the individual may be between about 4 μg/mL and about 50 μg/mL for a portion of the time that the intravesical drug delivery system is inserted into the bladder. The period in which the intravesical drug delivery system is inserted into the bladder can otherwise be referred to as an indwelling period. In some examples, the indwelling period is about three weeks, or between about 14-21 days. In some examples, the concentration of gemcitabine in the urine is at least 4-5 μg/mL for at least one week. In some examples, the concentration of gemcitabine in the urine is at a maximum between days 2 and 4 following insertion of an intravesical drug delivery system into the bladder of the individual.

In some examples, the intravesical drug delivery system releases gemcitabine at a zero-order release rate for an extended period, followed by a reduced, non-zero-order release rate over a decay period during each dosing cycle of gemcitabine. For example, the non-zero-order release rate may be a first-order release rate. The extended period may be from about day 0 to about day 7 or day 8. The decay period may be from about day 7 or about day 8 to about day 21.

In some examples, the intravesical drug delivery system may release the gemcitabine continuously or intermittently to achieve a concentration of the drug in the bladder that produces an extended, sustained, and/or therapeutically effective concentration of the drug in urine in the bladder as described in the methods provided herein. For example, the intravesical drug delivery system may release the gemcitabine in an amount of about 0.5 mg/day to about 50 mg/day, about 1 mg/day to about 40 mg/day, about 3 mg/day to about 30 mg/day, about 5 mg/day to about 20 mg/day, or about 1 mg/day to about 15 mg/day. These release rates can be provided over an indwelling period as described herein, such as an indwelling period of about three weeks. In some examples, about 70% of the gemcitabine is released in the first 7 days of the indwelling period. In some examples, about 90% of the gemcitabine is released by day 21 of the indwelling period.

During each dosing cycle, the individual may be instructed to drink at least about 1500 mL of fluid per day. For example, the individual may be instructed to drink at least about 500-2500 mL, 1000-2000 mL, or 1250-1750 mL of fluid per day.

In some examples, the methods of treatment may include administering anticholinergics, nonsteroidal anti-inflammatory drugs (NSAIDs), or bladder analgesics such as phenazopyridine prior to or after insertion or removal of the intravesical drug delivery system. In some examples, the methods of treatment may include administering prophylactic antibiotics with insertion and/or removal of the one of intravesical drug delivery system.

As described in greater detail below, in some examples, the intravesical drug delivery system can be inserted into the bladder transurethrally using a catheter. In some examples, the intravesical drug delivery system is removed using forceps and cystoscopy.

In some embodiments, the majority of gemcitabine within the intravesical drug delivery system is released in the first seven days of indwelling. In some embodiments, about 35% to about 55% of the 225 mg gemcitabine in the intravesical drug delivery system is released into the urine by day seven (after insertion into the bladder). In some embodiments, the intravesical drug delivery system releases gemcitabine at a slower rate following the first seven days of indwelling. In some embodiments, about 70% to about 90% of the 225 mg gemcitabine in the intravesical drug delivery system is released into the urine by day 21 (after insertion into the bladder). In some embodiments, about 3% to about 20% of the 225 mg gemcitabine in the intravesical drug delivery system is released each day for the first seven days. In some embodiments, about 3% to about 20% of the 225 mg gemcitabine in the intravesical drug delivery system is released each day for the first seven days. In some embodiments, the gemcitabine is at a subtherapeutic level in the urine after day 7. In some embodiments, the gemcitabine is at a subtherapeutic level in the urine after day 10.

In some embodiments, the majority of gemcitabine within the intravesical system is released in the first seven days of indwelling. In some embodiments, about 65% to about 85% of the gemcitabine in the intravesical system is released into urine in the individual's bladder over about the first 7 days after insertion into the bladder. In some embodiments, about 70% of the gemcitabine in the intravesical system is released into the urine over about the first 7 days after insertion into the bladder, wherein the percent of gemcitabine released is estimated using an in vitro release (IVR) model.

In some embodiments, about 76.9% of the gemcitabine in the intravesical system is released into the urine over about the first 7 days after insertion into the bladder, wherein the percent of gemcitabine released is estimated using in vivo data. In some embodiments, about 77% of the total gemcitabine dose in the intravesical system is released into the urine as gemcitabine and dFdU over about the first 7 days after insertion into the bladder, wherein the percent of gemcitabine released is estimated using in vivo data. In some embodiments, the in vivo data comprise urine gemcitabine related component (GRC) amount-time profiles, wherein the percent of gemcitabine released is estimated using nonlinear mixed effect modeling.

In some embodiments, the intravesical system releases gemcitabine at a slower rate following the first seven days of indwelling. In some embodiments, about 85% to about 100% of the gemcitabine is released over about 21 days after insertion into the bladder. In some embodiments, about 90% of the gemcitabine in the intravesical system is released into the urine over about the first 21 days after insertion into the bladder, wherein the percent of gemcitabine released is estimated using an in vitro release model. In some embodiments, about 99% of the gemcitabine in the intravesical system is released into the urine over about the first 21 days after insertion into the bladder, wherein the percent of gemcitabine released is estimated using in vivo data. In some embodiments, about 99% of the total gemcitabine dose in the intravesical system is released into the urine as gemcitabine and dFdU over about the first 21 days after insertion into the bladder, wherein the percent of gemcitabine released is estimated using in vivo data. In some embodiments, the in vivo data comprise urine gemcitabine related component (GRC) amount-time profiles, wherein the percent of gemcitabine released is estimated using nonlinear mixed effect modeling.

In some embodiments, the intravesical drug delivery system contains 225 mg of gemcitabine, and provides sustained release of gemcitabine into bladder urine, with approximately 70% released by Day 7 and approximately 90% released by Day 21.

In some embodiments, the majority of gemcitabine within the intravesical drug delivery system is released in the first seven days of indwelling. In some embodiments, about 35% to about 55% of the 256 mg gemcitabine HCL in the intravesical drug delivery system is released into the urine by day seven (after insertion into the bladder). In some embodiments, the intravesical drug delivery system releases gemcitabine at a slower rate following the first seven days of indwelling. In some embodiments, about 70% to about 90% of the 256 mg gemcitabine HCl in the intravesical drug delivery system is released into the urine by day 21 (after insertion into the bladder). In some embodiments, about 3% to about 20% of the 256 mg gemcitabine HCl in the intravesical drug delivery system is released each day for the first seven days. In some embodiments, the gemcitabine is at a subtherapeutic level in the urine after day 7. In some embodiments, the gemcitabine is at a subtherapeutic level in the urine after day 10.

### Endpoints

Methods of increasing event-free survival (EFS) for Bacillus Calmette-Guérin (BCG)-naïve high-risk non-muscle invasive bladder cancer (HR-NMIBC) in an individual are provided herein. Also provided herein are methods of increasing event-free survival (EFS) for Bacillus Calmette-Guérin (BCG)-naïve non-muscle invasive bladder cancer (NMIBC) in an individual. The methods can include any of the above methods of treatment described herein with respect to the first phase and/or the first phase and second phase. In some examples, the methods can include the additional third phase.

The treatment may result in an increased EFS of the individual compared to the EFS achieved by intravesical BCG. In some examples, the treatment results in an increased EFS in a population of patients receiving such therapy compared to a population of patients receiving intravesical BCG. In some embodiments, the treatment results in at least a 10% EFS increase. Intravesical BCG can include full dose induction intravesical BCG treatment once weekly for 6 weeks. The intravesical BCG treatment may be performed following a transurethral resection of bladder tumor (TURBT) procedure. In some examples, the methods of treatment include administering to the bladder of the individual a plurality of intravesical drug delivery systems during a first phase, each intravesical drug delivery system comprising about 225 mg of gemcitabine, wherein administering the plurality of intravesical drug delivery systems during the first phase comprises two or more three-week dosing cycles of gemcitabine, comprising: inserting one of the plurality of intravesical drug delivery systems into the bladder about once every three weeks (Q3W); and removing the intravesical drug delivery system from the bladder about three weeks after insertion, wherein in each dosing cycle of gemcitabine steps (i) and (ii) are repeated with a new one of the plurality of intravesical drug delivery systems for a total of up to six months. In some examples, the intravesical drug delivery system can comprise about 256 mg gemcitabine HCl (equivalent to about 225 mg gemcitabine free base). In some examples, the methods of treatment further include intravenously administering to the individual about 360 mg of cetrelimab about once every three weeks (Q3W) for at least six months.

In some examples, the methods of treatment may include extending EFS of the individual compared to the EFS achieved by intravesical BCG. In some examples, the methods of treatment may include extending EFS in a population of patients receiving such therapy compared to a population of patients receiving intravesical BCG. In some examples, the methods of treatment include administering to the bladder of the individual a plurality of intravesical drug delivery systems during a first phase, each intravesical drug delivery system comprising about 225 mg of gemcitabine, wherein administering the plurality of intravesical drug delivery systems during the first phase comprises two or more three-week dosing cycles of gemcitabine, comprising: inserting one of the plurality of intravesical drug delivery systems into the bladder about once every three weeks (Q3W); and removing the intravesical drug delivery system from the bladder about three weeks after insertion, wherein in each dosing cycle of gemcitabine steps (i) and (ii) are repeated with a new one of the plurality of intravesical drug delivery systems for a total of up to six months. In some examples, the intravesical drug delivery system can comprise about 256 mg gemcitabine HCl (equivalent to about 225 mg gemcitabine free base). In some examples, the methods of treatment further include intravenously administering to the individual about 360 mg of cetrelimab about once every three weeks (Q3W) for at least six months.

Event-free survival (EFS) can be measured as the time from randomization to either the time of the first recurrence of high-risk disease, progression, or death due to any cause, whichever occurs first. Progression can be defined as: 1) an increase of stage from Ta to T1 or from CIS to T1, or 2) progression to MIBC (T>2) or to lymph node (N+) or to distant disease (M+), whichever occurs first. For participants with CIS, persistent disease at 6 months (Week 24) can also be considered an EFS event. The EFS endpoint can be determined by a combination of local and/or central disease assessments including local cytology, local cystoscopy, central pathology, or central imaging as applicable. An EFS event can be defined by 1) a positive local urine cytology, or 2) a positive local cystoscopy with confirmatory centrally reviewed biopsy proven high-risk disease (high grade papillary Ta, any T1 or CIS disease), or 3) a centrally reviewed imaging proven disease progression/metastasis as applicable. In some embodiments, EFS is measured from the start of treatment.

In some examples, overall complete response (CR) rate, recurrence-free survival (RFS), time to progression (TTP), and/or overall survival (OS) are improved.

In some examples, the methods of treatment can result in an increased overall complete response (CR) rate in a population of patients who have received the treatment compared to the CR rate achieved by intravesical BCG. Overall CR rate can be measured by determining the proportion of participants with CIS who have no presence of high-risk disease at 6 months following randomization or from start of treatment. In some examples, the methods of treatment may include increasing an overall CR rate in a population of patients receiving such therapy compared to a population of patients receiving intravesical BCG. In some examples, the methods of treatment include administering to the bladder of the individual a plurality of intravesical drug delivery systems during a first phase, each intravesical drug delivery system comprising about 225 mg of gemcitabine, wherein administering the plurality of intravesical drug delivery systems during the first phase comprises two or more three-week dosing cycles of gemcitabine, comprising: inserting one of the plurality of intravesical drug delivery systems into the bladder about once every three weeks (Q3W); and removing the intravesical drug delivery system from the bladder about three weeks after insertion, wherein in each dosing cycle of gemcitabine steps (i) and (ii) are repeated with a new one of the plurality of intravesical drug delivery systems for a total of up to six months. In some examples, the intravesical drug delivery system can comprise about 256 mg gemcitabine HCL (equivalent to about 225 mg gemcitabine free base). In some examples, the methods of treatment further include intravenously administering to the individual about 360 mg of cetrelimab about once every three weeks (Q3W) for at least six months.

In some examples, the methods of treatment can result in an increased duration of CR in an individual compared to the duration of CR achieved by intravesical BCG. Duration of CR can be defined from the time of first CR achieved to first evidence of recurrence, progression or death due to any cause (whichever occurs first) for participants who achieve a CR. In some examples, the methods of treatment can result in an increased duration of CR in a population of patients receiving such therapy compared to a population of patients receiving intravesical BCG. In some examples, the methods of treatment may include increasing a duration of CR of the individual compared to the duration of CR achieved by intravesical BCG. In some examples, the methods of treatment may include increasing a duration of CR in a population of patients receiving such therapy compared to a population of patients receiving intravesical BCG. In some examples, the methods of treatment include administering to the bladder of the individual a plurality of intravesical drug delivery systems during a first phase, each intravesical drug delivery system comprising about 225 mg of gemcitabine, wherein administering the plurality of intravesical drug delivery systems during the first phase comprises two or more three-week dosing cycles of gemcitabine, comprising: inserting one of the plurality of intravesical drug delivery systems into the bladder about once every three weeks (Q3W); and removing the intravesical drug delivery system from the bladder about three weeks after insertion, wherein in each dosing cycle of gemcitabine steps (i) and (ii) are repeated with a new one of the plurality of intravesical drug delivery systems for a total of up to six months. In some examples, the intravesical drug delivery system can comprise about 256 mg gemcitabine HCl (equivalent to about 225 mg gemcitabine free base). In some examples, the methods of treatment further include intravenously administering to the individual about 360 mg of cetrelimab about once every three weeks (Q3W) for at least six months.

In some examples, the methods of treatment can result in an increased recurrence-free survival (RFS) in an individual compared to the RFS achieved by intravesical BCG. RFS can be measured as the time from randomization to the time of the first recurrence of high-risk disease, or death due to any cause, whichever occurs first. In some examples, RFS is measured from the start of treatment. In some examples, the methods of treatment can result in an increased RFS in a population of patients receiving such therapy compared to a population of patients receiving intravesical BCG. In some examples, the methods of treatment may include increasing an RFS of the individual compared to the RFS achieved by intravesical BCG. In some examples, the methods of treatment may include increasing an RFS in a population of patients receiving such therapy compared to a population of patients receiving intravesical BCG. In some examples, the methods of treatment include administering to the bladder of the individual a plurality of intravesical drug delivery systems during a first phase, each intravesical drug delivery system comprising about 225 mg of gemcitabine, wherein administering the plurality of intravesical drug delivery systems during the first phase comprises two or more three-week dosing cycles of gemcitabine, comprising: inserting one of the plurality of intravesical drug delivery systems into the bladder about once every three weeks (Q3W); and removing the intravesical drug delivery system from the bladder about three weeks after insertion, wherein in each dosing cycle of gemcitabine steps (i) and (ii) are repeated with a new one of the plurality of intravesical drug delivery systems for a total of up to six months. In some examples, the intravesical drug delivery system can comprise about 256 mg gemcitabine HCL. In some examples, the methods of treatment further include intravenously administering to the individual about 360 mg of cetrelimab about once every three weeks (Q3W) for at least six months.

In some examples, the methods of treatment can result in an increased time to progression (TTP) in an individual compared to the TTP achieved by intravesical BCG. TTP can be measured as the time from randomization to the date of first documented evidence of disease progression or death due to disease progression, whichever occurs first. In some examples, TTP is measured from the start of treatment. In some examples, the methods of treatment can result in an increased TTP in a population of patients receiving such therapy compared to a population of patients receiving intravesical BCG. In some examples, the methods of treatment may include increasing a TTP of the individual compared to the TTP achieved by intravesical BCG. In some examples, the methods of treatment may include increasing a TTP in a population of patients receiving such therapy compared to a population of patients receiving intravesical BCG. In some examples, the methods of treatment include administering to the bladder of the individual a plurality of intravesical drug delivery systems during a first phase, each intravesical drug delivery system comprising about 225 mg of gemcitabine, wherein administering the plurality of intravesical drug delivery systems during the first phase comprises two or more three-week dosing cycles of gemcitabine, comprising: inserting one of the plurality of intravesical drug delivery systems into the bladder about once every three weeks (Q3W); and removing the intravesical drug delivery system from the bladder about three weeks after insertion, wherein in each dosing cycle of gemcitabine steps (i) and (ii) are repeated with a new one of the plurality of intravesical drug delivery systems for a total of up to six months. In some examples, the intravesical drug delivery system can comprise about 256 mg gemcitabine HCL. In some examples, the methods of treatment further include intravenously administering to the individual about 360 mg of cetrelimab about once every three weeks (Q3W) for at least six months.

In some examples, the methods of treatment can result in an increased overall survival (OS) in an individual compared to the OS achieved by intravesical BCG. OS can be defined as the time from randomization to death, due to any cause. In some examples, OS is measured from the start of treatment. In some examples, the methods of treatment can result in an increased OS in a population of patients receiving such therapy compared to a population of patients receiving intravesical BCG. In some examples, the methods of treatment may include increasing an OS of the individual compared to the OS achieved by intravesical BCG. In some examples, the methods of treatment may include increasing an OS in a population of patients receiving such therapy compared to a population of patients receiving intravesical BCG. In some examples, the methods of treatment include administering to the bladder of the individual a plurality of intravesical drug delivery systems during a first phase, each intravesical drug delivery system comprising about 225 mg of gemcitabine, wherein administering the plurality of intravesical drug delivery systems during the first phase comprises two or more three-week dosing cycles of gemcitabine, comprising: inserting one of the plurality of intravesical drug delivery systems into the bladder about once every three weeks (Q3W); and removing the intravesical drug delivery system from the bladder about three weeks after insertion, wherein in each dosing cycle of gemcitabine steps (i) and (ii) are repeated with a new one of the plurality of intravesical drug delivery systems for a total of up to six months. In some examples, the intravesical drug delivery system can comprise about 256 mg gemcitabine HCL. In some examples, the methods of treatment further include intravenously administering to the individual about 360 mg of cetrelimab about once every three weeks (Q3W) for at least six months.

In some examples, the methods of treatment can result in an increased cancer specific survival (CSS) in an individual compared to the CSS achieved by intravesical BCG. CSS can be measured as the time from randomization to the date of death due to bladder cancer. In some examples, CSS is measured from the start of treatment. In some examples, the methods of treatment can result in an increased CSS in a population of patients receiving such therapy compared to a population of patients receiving intravesical BCG. In some examples, the methods of treatment may include increasing a CSS of the individual compared to the CSS achieved by intravesical BCG. In some examples, the methods of treatment may include increasing a CSS in a population of patients receiving such therapy compared to a population of patients receiving intravesical BCG. In some examples, the methods of treatment include administering to the bladder of the individual a plurality of intravesical drug delivery systems during a first phase, each intravesical drug delivery system comprising about 225 mg of gemcitabine, wherein administering the plurality of intravesical drug delivery systems during the first phase comprises two or more three-week dosing cycles of gemcitabine, comprising: inserting one of the plurality of intravesical drug delivery systems into the bladder about once every three weeks (Q3W); and removing the intravesical drug delivery system from the bladder about three weeks after insertion, wherein in each dosing cycle of gemcitabine steps (i) and (ii) are repeated with a new one of the plurality of intravesical drug delivery systems for a total of up to six months. In some examples, the intravesical drug delivery system can comprise about 256 mg gemcitabine HCL. In some examples, the methods of treatment further include intravenously administering to the individual about 360 mg of cetrelimab about once every three weeks (Q3W) for at least six months.

In some examples, the methods of treatment can result in improved safety and tolerability for the individual compared to the safety and tolerability of intravesical BCG. Safety and tolerability may be measured by the number of participants with adverse events (AEs) by grades according to Common Terminology Criteria for Adverse Events (CTCAE) version 5.0. In some examples, the methods of treatment can result in increased safety and tolerability for a population of patients receiving such therapy compared to a population of patients receiving intravesical BCG. In some examples, the methods of treatment may include increasing safety and tolerability for the individual compared to the safety and tolerability achieved by intravesical BCG. In some examples, the methods of treatment may include increasing safety and tolerability for a population of patients receiving such therapy compared to a population of patients receiving intravesical BCG. In some examples, the methods of treatment include administering to the bladder of the individual a plurality of intravesical drug delivery systems during a first phase, each intravesical drug delivery system comprising about 225 mg of gemcitabine, wherein administering the plurality of intravesical drug delivery systems during the first phase comprises two or more three-week dosing cycles of gemcitabine, comprising: inserting one of the plurality of intravesical drug delivery systems into the bladder about once every three weeks (Q3W); and removing the intravesical drug delivery system from the bladder about three weeks after insertion, wherein in each dosing cycle of gemcitabine steps (i) and (ii) are repeated with a new one of the plurality of intravesical drug delivery systems for a total of up to six months. In some examples, the intravesical drug delivery system can comprise about 256 mg gemcitabine HCL. In some examples, the methods of treatment further include intravenously administering to the individual about 360 mg of cetrelimab about once every three weeks (Q3W) for at least six months.

In some examples, the methods of treatment can result in improved health-related quality of life (HRQOL) compared to the HRQOL of intravesical BCG. HRQOL can be measured by a change from baseline and time to symptom deterioration in European Organization for Research and Treatment of Cancer-Non-muscle Invasive Bladder Cancer 24 (EORTC-QLQ-NMIBC24). In some examples, the methods of treatment include administering to the bladder of the individual a plurality of intravesical drug delivery systems during a first phase, each intravesical drug delivery system comprising about 225 mg of gemcitabine, wherein administering the plurality of intravesical drug delivery systems during the first phase comprises two or more three-week dosing cycles of gemcitabine, comprising: inserting one of the plurality of intravesical drug delivery systems into the bladder about once every three weeks (Q3W); and removing the intravesical drug delivery system from the bladder about three weeks after insertion, wherein in each dosing cycle of gemcitabine steps (i) and (ii) are repeated with a new one of the plurality of intravesical drug delivery systems for a total of up to six months. In some examples, the intravesical drug delivery system can comprise about 256 mg gemcitabine HCL. In some examples, the methods of treatment further include intravenously administering to the individual about 360 mg of cetrelimab about once every three weeks (Q3W) for at least six months.

In some examples, the frequency and grade of adverse events (AEs) in a population of patients who have received therapies described herein is lower compared to intravesical instillation, and, more generally, the standard of care (SOC) for treating BCG-naïve HR-NMIBC and/or NMIBC. In some examples, the method comprises decreasing AEs in a population of patients. AEs can be defined according to Common Terminology Criteria for Adverse Events [CTCAE] version 5.0 and according to Patient-Reported Outcomes version of the Common Terminology Criteria for Adverse Events (NCI PRO-CTCAE). In some examples, the methods of treatment include administering to the bladder of the individual a plurality of intravesical drug delivery systems during a first phase, each intravesical drug delivery system comprising about 225 mg of gemcitabine, wherein administering the plurality of intravesical drug delivery systems during the first phase comprises two or more three-week dosing cycles of gemcitabine, comprising: inserting one of the plurality of intravesical drug delivery systems into the bladder about once every three weeks (Q3W); and removing the intravesical drug delivery system from the bladder about three weeks after insertion, wherein in each dosing cycle of gemcitabine steps (i) and (ii) are repeated with a new one of the plurality of intravesical drug delivery systems for a total of up to six months. In some examples, the intravesical drug delivery system can comprise about 256 mg gemcitabine HCL. In some examples, the methods of treatment further include intravenously administering to the individual about 360 mg of cetrelimab about once every three weeks (Q3W) for at least six months.

In some examples, the methods described herein result in Grade 3 or greater treatment emergent adverse events (TEAEs) in less than 85% of patients. In some examples, the methods described herein result in TEAEs related to an intravesical drug delivery system in less than 10% of patients. In some examples, the methods of treatment include administering to the bladder of the individual a plurality of intravesical drug delivery systems during a first phase, each intravesical drug delivery system comprising about 225 mg of gemcitabine, wherein administering the plurality of intravesical drug delivery systems during the first phase comprises two or more three-week dosing cycles of gemcitabine, comprising: inserting one of the plurality of intravesical drug delivery systems into the bladder about once every three weeks (Q3W); and removing the intravesical drug delivery system from the bladder about three weeks after insertion, wherein in each dosing cycle of gemcitabine steps (i) and (ii) are repeated with a new one of the plurality of intravesical drug delivery systems for a total of up to six months. In some examples, the intravesical drug delivery system can comprise about 256 mg gemcitabine HCL. In some examples, the methods of treatment further include intravenously administering to the individual about 360 mg of cetrelimab about once every three weeks (Q3W) for at least six months.

In some examples, the methods described herein result in serious adverse events (SAEs) in less than 10% of patients. In some examples, the methods described herein result in SAEs related to an intravesical drug delivery system in less than 5% of patients. In some examples, the methods of treatment include administering to the bladder of the individual a plurality of intravesical drug delivery systems during a first phase, each intravesical drug delivery system comprising about 225 mg of gemcitabine, wherein administering the plurality of intravesical drug delivery systems during the first phase comprises two or more three-week dosing cycles of gemcitabine, comprising: inserting one of the plurality of intravesical drug delivery systems into the bladder about once every three weeks (Q3W); and removing the intravesical drug delivery system from the bladder about three weeks after insertion, wherein in each dosing cycle of gemcitabine steps (i) and (ii) are repeated with a new one of the plurality of intravesical drug delivery systems for a total of up to six months. In some examples, the intravesical drug delivery system can comprise about 256 mg gemcitabine HCL. In some examples, the methods of treatment further include intravenously administering to the individual about 360 mg of cetrelimab about once every three weeks (Q3W) for at least six months.

In some examples, time to cystectomy (TTC), metastasis-free survival (MFS), all-cause EFS (aEFS), and/or EFS2 are improved.

In some examples, the methods of treatment can result in an increased time to cystectomy (TTC) in a population of patients who have received the treatment compared to the TTC achieved by intravesical BCG. TTC can be measured from the date of randomization to the date of the cystectomy. In some examples, the methods of treatment may include increasing TTC in a population of patients receiving such therapy compared to a population of patients receiving intravesical BCG.

In some examples, the methods of treatment can result in an increased metastasis-free survival (MFS) in a population of patients who have received the treatment compared to the MFS achieved by intravesical BCG. MFS can be defined as the time from randomization to first radiologic or histologic evidence of metastatic disease or death due to any cause. In some examples, the methods of treatment may include increasing MFS in a population of patients receiving such therapy compared to a population of patients receiving intravesical BCG.

In some examples, the methods of treatment can result in an increased all-cause event-free survival (aEFS) in a population of patients who have received the treatment compared to the aEFS achieved by intravesical BCG. All-cause EFS (aEFS) can include EFS events and recurrence of non-high-risk NMIBC, and can be measured as the time from randomization to either the time of EFS or any recurrence of NMIBC, whichever occurs first. In some examples, the methods of treatment may include increasing aEFS in a population of patients receiving such therapy compared to a population of patients receiving intravesical BCG.

In some examples, the methods of treatment can result in an increased event-free survival 2 (EFS2) in a population of patients who have received the treatment compared to the EFS2 achieved by intravesical BCG. EFS2 can be defined as the time from randomization until the date of high-risk recurrence, progression, or death due to any cause on the first subsequent non-surgical anticancer treatment, whichever occurs first. In some examples, the methods of treatment may include increasing EFS2 in a population of patients receiving such therapy compared to a population of patients receiving intravesical BCG.

### Patient Populations

In some examples, the individual or population of patients has NMIBC. In some examples, the individual or population of patients has HR-NMIBC. In some examples, the individual or population of patients has Bacillus Calmette-Guérin (BCG)-naïve NMIBC. In some examples, the individual or population of patients has Bacillus Calmette-Guérin (BCG)-naïve HR-NMIBC. Kamat et al. (2016 J. Clinical Oncology) provides a definition for BCG-naïve HR-NMIBC. Specifically, Kamat's definition provides that individuals with BCG-naïve HR-NMIBC have histologically confirmed T1 and/or high-grade tumor and/or carcinoma in situ (CIS) that has never been treated with BCG immunotherapy and could include patients who previously received but stopped BCG more than 3 years before study entry. Kamat indicates that patients who previously received but stopped BCG more than 3 years before study entry can be considered BCG naïve because clinical experience suggests that response rates in these patients are similar to those in BCG-naïve patients. Accordingly, in some examples, BCG-naïve includes individuals who have not received previous intravesical BCG or who previously received but stopped BCG more than 3 years before the start of treatment. In some examples, the individual or population of patients can have high-grade Ta stage bladder cancer. The individual or population of patients can have T1 stage bladder cancer. A urothelial carcinoma of the bladder is staged as “high-grade Ta” when the tumors are non-invasive papillary carcinomas. A urothelial carcinoma of the bladder is staged as “T1” when the tumors have grown into the connective tissue layer of the bladder wall, but it has not reached the layer of muscle in the bladder wall. The individual or population of patients can have carcinoma in situ (CIS). The individual or population of patients can have a mixed histology tumor with predominant urothelial differentiation. The individual or population of patients can have an Eastern Cooperative Oncology Group (ECOG) performance status Grade 0, 1, or 2. The individual or population of patients can have papillary disease, and the papillary disease can be fully resected prior to the treatment with gemcitabine.

In some examples, the individual or population of patients may not have a presence or history of histologically confirmed, muscle invasive, locally advanced, nonresectable, or metastatic urothelial carcinoma of stage T2 or greater. The individual or population of patients may not have urothelial carcinoma or a histological variant at any site outside of the bladder. The individual or population of patients may not have a history of HR-NMIBC less than three years from current diagnosis. In some examples, the individual or population of patients may not have a history of NMIBC less than three years from current diagnosis. The individual or population of patients may not have visible papillary disease at the time of diagnosis. The individual or population of patients may not have N+ and/or M+ bladder cancer. The individual or population of patients may not have neuroendocrine, micropapillary, signet ring cell, plasmacytoid, or sarcomatoid features. The individual or population of patients may not have a presence of any bladder or urothelial anatomical feature that prevents the safe insertion, indwelling use, and removal of the intravesical drug delivery system. The individual or population of patients may not have a history of clinically significant polyuria with recorded 24-hour urine volumes greater than 4000 mL. The individual or population of patients may not have received BCG within three years from diagnosis. In some examples, all visible papillary disease is fully resected prior to treatment with gemcitabine. In some examples, the individual or population of patients has undergone a transurethral resection of bladder tumor (TURBT) and/or bladder biopsy prior to treatment with gemcitabine. For example, the TURBT/bladder biopsy may have been performed within about 90 days prior to the start of treatment with gemcitabine.

In some embodiments, the individual is elderly. In some embodiments, the individual is at least 65 years old. In some embodiments, the individual is between about 65 to about 70, about 70 to about 75, about 75 to about 80, about 80 to about 85, or about 85 to about 90 years old. In some embodiments, the individual is frail.

### Intravesical Drug Delivery Systems

The various aspects and embodiments described in this section in the context of a method of treatment comprising administering gemcitabine using an intravesical drug delivery system also apply to gemcitabine for use according to the methods described herein, and minitablets comprising gemcitabine for use according to the methods described herein. In particular, the aspects and embodiments described in this section also relate to gemcitabine for use with an intravesical drug delivery system for use in the methods provided herein, wherein the method comprises continuous delivery of gemcitabine to the bladder by an intravesical drug delivery system comprising the intravesical device, and to gemcitabine for use according to the methods described herein, wherein the gemcitabine is administered by an intravesical drug delivery system.

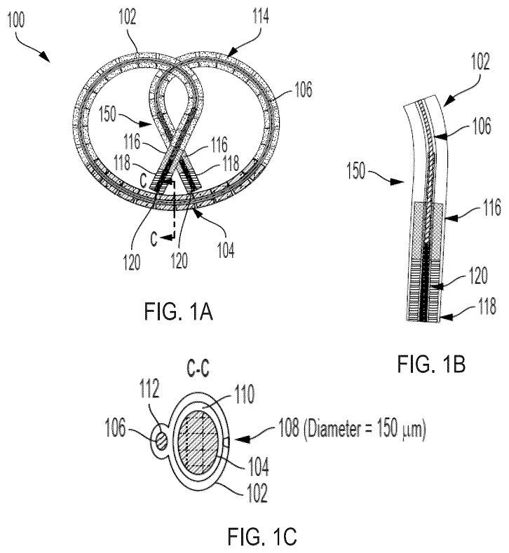

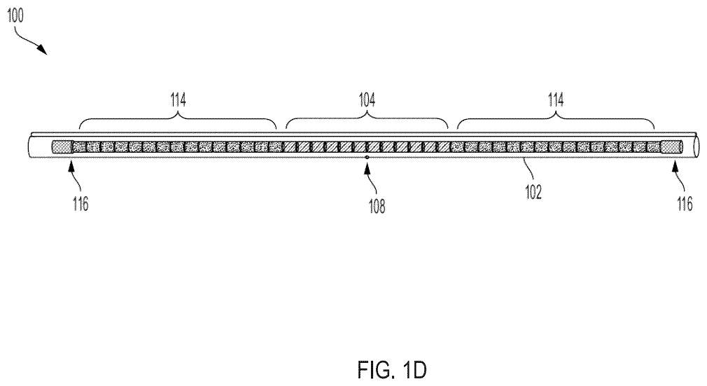

As described herein, administering gemcitabine can include the use of an intravesical drug delivery system that comprises the gemcitabine and is insertable into the bladder for extended or sustained release of the gemcitabine. An exemplary intravesical drug delivery system 100 is shown in and described below with respect to FIGS. 1A-1D and may otherwise be referred to herein as “TAR-200” or “gemcitabine targeted releasing system”. When inserted into the bladder, the intravesical drug delivery system is submerged and freely mobile in the urine during the indwelling period.

In some embodiments, the intravesical drug delivery system is a sterile, non-resorbable, flexible, intravesical system containing 256 mg gemcitabine HCl (equivalent to 225 mg gemcitabine free base). Once administered, the intravesical drug delivery system retains its bi-oval shape and is free-moving within the bladder.

Gemcitabine HCl (a nucleoside metabolic inhibitor) is also called 2′-deoxy-2′,2′-difluorocytidine monohydrochloride (β-isomer). The molecular formula for gemcitabine HCL is C₉H₁₁F₂N₃O₄·HCl, the molecular weight is 299.66, and the structural formula is:

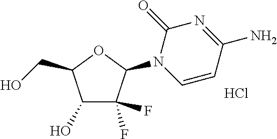

In some embodiments, the intravesical drug delivery system comprises an almost white to light pink-brown colored gemcitabine component at the center surrounded on each side by off white to light blue-colored osmotic components. In some embodiments, the intravesical drug delivery system comprises gemcitabine minitablets (which may appear as solid material) and osmotic minitablets (for osmotic performance), and are contained within the bi-oval shaped device constituent. The color of the minitablets may vary but that has no effect on the potency of the intravesical drug delivery system. In some embodiments, the gemcitabine component is an almost white to light pink-brown color. In some embodiments, the osmotic components are an off white to light blue-color. The minitablets contain the following ingredients:

Gemcitabine minitablets: containing a total dose of 225 mg of the active ingredient gemcitabine (as free base equivalent), and the following inactive ingredients: polyethylene glycol 8000 (8 mg), povidone K30 (13.4 mg), urea (42.6 mg).

Osmotic (urea) minitablets: contain no active ingredient, and the following inactive ingredients: urea (648 mg), polyethylene oxide 600,000 (72 mg), FD&C Blue No. 1 (0.0042 mg).

The bi-oval shaped tube consists of a dual lumen silicone part, a superelastic nitinol wire in a predefined shape (wireform), silicone spacers, and silicone adhesive. The minitablets are surrounded by a semipermeable membrane that contains a single delivery orifice. In some embodiments, the intravesical drug delivery system's coiled dimensions are approximately 6 cm wide×5 cm high. In some embodiments, the intravesical drug delivery system's coiled dimensions are approximately 5.5 cm wide×4.5 cm high.

The intravesical drug delivery system 100 can include a bi-oval shaped housing 102 that contains the gemcitabine 104. The housing 102 can be flexible for insertion and removal of the intravesical drug delivery system 100 into and from the bladder. For example, the housing 102 may be composed of a biocompatible elastomeric polymer (e.g., silicone, thermoplastic polyurethane (TPU), etc.) that has the desired mechanical properties for manipulating the intravesical drug delivery system 100. The housing 102 may be elastically deformable between a retention shape (e.g., a coiled or bi-oval shape, shown in FIG. 1A) for retaining the drug delivery system in the individual's bladder and a deployment shape (e.g., an uncoiled shape, shown in FIG. 1D) for passing the drug delivery system through the individual's urethra (e.g., during insertion and removal of the intravesical drug delivery system 100). For example, the intravesical drug delivery system 100 can include a retention frame 106 disposed within the housing 102 that has a predefined shape to retain the system in its bi-oval shape once inserted into the bladder. In some examples, the retention frame 106 is a super-elastic metal alloy wire, such as a nitinol wire. Once inserted into the bladder, the intravesical drug delivery system 100 may be moveable within the bladder (e.g., within the urine in the bladder).

As illustrated in FIG. 1C, the housing 102 can include a dual lumen composed of a drug reservoir lumen 110 (e.g., a large lumen) that contains the gemcitabine 104 and a retention frame lumen 112 (e.g., a smaller lumen) that contains the retention frame 106. The drug reservoir lumen 110 may have an inner diameter between about 2 mm and 3 mm. The retention frame lumen 112 may have an inner diameter between about 0.4 mm and 1 mm. Although illustrated as two separate but attached (e.g., monolithic) circular portions in FIG. 1C, it is to be understood that the housing 102 may embody any reasonable shape that includes a dual lumen to separately contain the gemcitabine 104 and the retention frame 106. For example, the outer-most walls of the housing 102 may have a substantially circular or ovular shape which includes the dual lumen.

The intravesical drug delivery system 100 may comprise about 225 mg of gemcitabine. In some examples, the intravesical drug delivery system can comprise about 256 mg of gemcitabine hydrochloride (HCl) (equivalent to about 225 mg gemcitabine free base). The gemcitabine 104 contained within the intravesical drug delivery system 100 may be in a non-liquid form. For example, the gemcitabine may be in the form of minitablets. As shown in FIGS. 1A and 1D, the gemcitabine minitablets 104 can be serially arranged within the housing 102.

The intravesical drug delivery system 100 can include a single delivery orifice 108 that controllably releases the gemcitabine 104 from the drug reservoir lumen 110 through the delivery orifice 108. The delivery orifice 108 may be a laser-drilled delivery orifice. In some examples, the diameter of the delivery orifice 108 can be between about 25-300 μm, such as between about 50-250 μm, or about 100-200 μm (e.g., about 150 μm). In FIG. 1A, the delivery orifice 108 is positioned centrally between the two ovals making up the bi-oval shape of the system for maximum, controlled release of the gemcitabine 104 from the delivery orifice 108, as described in greater detail below.

The intravesical drug delivery system 100 may be configured to release the gemcitabine 104 into the urine in the bladder by osmotic pressure generated by an osmotic agent 114 (e.g., urea) contained within the housing 102 of the intravesical drug delivery system 100. For example, the drug reservoir lumen 110 may contain a first unit (e.g., a first minitablet) that includes the gemcitabine 104 and a second unit (e.g., a second minitablet) that includes the osmotic agent 114. As shown in FIG. 1A, the housing 102 may contain a plurality of first units (e.g., minitablets) including gemcitabine 104 and a plurality of second units (e.g., minitablets) including osmotic agent 114. For example, the housing 102 may contain between about 30-50, e.g., about 35-45, about 38-42, or about 42 total minitablets. In some examples, the housing 102 may contain about 25-35, e.g., about 28-32 or about 30 total osmotic minitablets. In some examples, the housing may contain about 8-15, e.g., about 10-14 or about 12 gemcitabine minitablets.

The minitablets (including the gemcitabine minitablets and the osmotic minitablets) may be arranged serially within the housing 102, as shown at least in FIG. 1D. In the arrangement illustrated in FIG. 1D, the plurality of minitablets including gemcitabine 104 are flanked on either side by the plurality of minitablets including osmotic agent 114. This arrangement of gemcitabine minitablets and osmotic minitablets may be beneficial controlling and/or maximizing osmotic release of the gemcitabine 104 from the delivery orifice 108.

In some examples, the first unit including the gemcitabine 104 may further include an osmotic agent (i.e., in addition to or in place of the second unit including osmotic agent). The osmotic agent may include urea. In some examples, the first unit may include at least 50 percent by weight gemcitabine, at least 60 percent by weight gemcitabine, at least 75 percent by weight gemcitabine, from about 60 to about 99 percent by weight gemcitabine, or from about 75 to about 95 percent by weight gemcitabine.

In some examples, the second unit may include at least 80 percent by weight osmotic agent, at least 85 percent by weight osmotic agent, at least 90 percent by weight osmotic agent, from about 80 to about 99 percent by weight osmotic agent, or from about 85 to about 95 percent by weight osmotic agent. The remainder of the first unit and/or second unit may include excipients such as pharmaceutical lubricants, stabilizing agents, or binding agents, for example oil-based lubricants, polyethylene glycol (PEG), or polyvinylpyrrolidone (PVP). The excipients may also include a viscosity enhancing (e.g., release delay) agent, for example polyethylene oxide (PEO). For example, a viscosity enhancing agent could be provided in a portion of the first unit, a portion of the second unit, or both the first and second unit to further control release of the gemcitabine. In some embodiments, the first unit comprises 80% by weight gemcitabine HCl, 13.3% by weight urea, 2.5% by weight polyethylene glycol (PEG), and 4.2% by weight polyvinylpyrrolidone. In some embodiments, the PEG comprises PEG 8000.

In some embodiments, the second unit may include about 90 percent by weight urea and about 10 percent by weight oil-based pharmaceutical lubricant LUBRITAB®. In some embodiments, the second unit may include about 90 percent by weight urea and about 10 percent by weight polyethylene oxide (PEO). In a particular embodiment, the first unit contains at least 75 percent by weight gemcitabine HCl, such as about 80 percent by weight gemcitabine HCL. In a particular embodiment, the second unit contains at least 85 percent by weight urea, such as about 90 percent by weight urea.

The housing 102 may be formed of a semi-permeable membrane that is permeable to water/urine and impermeable to the gemcitabine 104 and osmotic agent 114 contained therein. In this manner, substantially no amount of the solubilized gemcitabine 104 or the osmotic agent 114 can diffuse through the housing 102 over the dosing cycle in which the intravesical drug delivery system 100 is inserted into the bladder. When inserted into the bladder, the intravesical drug delivery system 100 can operate as an osmotic pump. Water or urine in the individual's bladder can diffuse through the housing 102 into the drug reservoir lumen 110 to contact and solubilize the minitablets of gemcitabine 104 and minitablets of osmotic agent 114, creating an osmotic pressure to drive the solubilized drug from the drug delivery system through delivery orifice 108.

As shown in FIGS. 1A-1B the end portions 150 of, the intravesical drug delivery system 100 can include silicone spacers 116 disposed at each end of the housing 102 to close or plug the drug reservoir lumen 110. In some embodiments, the opposed ends of the retention frame 106 can overlay the respective silicone spacers 116. In some examples, a silicone adhesive 118 can be injected into the drug reservoir lumen 110 to secure the silicone spacers 116 within the ends of the drug reservoir lumen 110. Separately, the retention frame lumen 112 can be sealed using a silicone adhesive 120. Once the silicone adhesive is injected to the housing 102, any extra housing 102 at the ends can be trimmed.

As noted above, the intravesical drug delivery system 100 may be flexible between a bi-oval, or coiled, shape (retention shape), and an uncoiled shape (insertion shape). In the coiled shape, the intravesical drug delivery system 100 may have a width of about 4-7 cm, about 4.5-6.5 cm, or about 5-6 cm (e.g., 5.5 cm or 6 cm). In the coiled shape, the intravesical drug delivery system 100 may have a height of about 3-6 cm, about 3.5-5.5 cm, or about 4-5 cm (e.g., about 4.5 cm or 5 cm). In the uncoiled shape, the intravesical drug delivery system 100 may have a length between about 15-20 cm, such as about 16-19 cm or about 17-18 cm (e.g., 17 cm).

The intravesical drug delivery system 100 can be deployed into the bladder through the working channel of a catheter, cystoscope, or other deployment instrument positioned in the urethra. The intravesical drug delivery system 100 can be provided as part of a kit with a urinary placement catheter that facilitates deployment of the intravesical drug delivery system 100 into the bladder of an individual. An exemplary urinary placement catheter is described in U.S. Pat. No. 10,064,980, which is incorporated herein by reference in its entirety. The intravesical drug delivery system 100 can be removed from the bladder transurethrally via cystoscopy and endoscopic graspers. Additional details about the intravesical drug delivery system can be found in U.S. Pat. Nos. 10,729,823 and 11,020,575, the entire contents of which are expressly incorporated herein by reference.

### Conclusion

The foregoing description, for the purpose of explanation, has been described with reference to specific embodiments. However, the illustrative discussions above are not intended to be exhaustive or to limit the invention to the precise forms disclosed. Many modifications and variations are possible in view of the above teachings. The embodiments were chosen and described in order to best explain the principles of the techniques and their practical applications. Others skilled in the art are thereby enabled to best utilize the techniques and various embodiments with various modifications as are suited to the particular use contemplated.

Although the disclosure and examples have been fully described with reference to the accompanying figures, it is to be noted that various changes and modifications will become apparent to those skilled in the art. Such changes and modifications are to be understood as being included within the scope of the disclosure and examples as defined by the claims.

The entire disclosure of the patents and publications referred in this application are hereby incorporated herein by reference for all purposes. To the extent that any reference incorporated by reference conflicts with the instant disclosure, the instant disclosure shall control.

The numerical ranges disclosed inherently support any range or value within the disclosed numerical ranges, including the endpoints, even though a precise range limitation is not stated verbatim in the specification because this disclosure can be practiced throughout the disclosed numerical ranges.

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

The various aspects and embodiments described herein in the context of a method of treatment also apply to gemcitabine for use according to the methods described herein, and minitablets comprising gemcitabine for use according to the methods described herein, unless indicated otherwise. Similarly, the various aspects and embodiments described herein in the context of a method of treatment also apply to use of an intravesical drug delivery system comprising gemcitabine, according to the methods described herein.

### Embodiments

The following embodiments are exemplary and are not intended to limit the scope of any invention described herein.

1. A method of treating Bacillus Calmette-Guérin (BCG)-naïve high-risk non-muscle invasive bladder cancer (HR-NMIBC) in an individual, comprising: inserting an intravesical drug delivery system comprising about 225 mg of gemcitabine into the bladder of the individual about once every three weeks (Q3W) for a total of up to six months following starting treatment, wherein each intravesical drug delivery system is removed from the bladder about three weeks after insertion into the bladder and wherein each intravesical drug delivery system is removed prior to insertion of another intravesical drug delivery system.

2. A method of increasing event-free survival (EFS) for Bacillus Calmette-Guérin (BCG)-naïve high-risk non-muscle invasive bladder cancer (HR-NMIBC) in an individual, comprising: inserting an intravesical drug delivery system comprising about 225 mg of gemcitabine into the bladder of the individual about once every three weeks (Q3W) for a total of up to six months following starting treatment, wherein each intravesical drug delivery system is removed from the bladder about three weeks after insertion into the bladder and wherein each intravesical drug delivery system is removed prior to insertion of another intravesical drug delivery system, and wherein the method results in an increased event-free survival (EFS) of the individual compared to the EFS achieved by intravesical BCG, and/or wherein the method results in an increased EFS in a population of patients receiving such therapy compared to a population of patients receiving intravesical BCG.

3. The method of embodiment 1 or 2, comprising inserting an intravesical drug delivery system comprising about 225 mg of gemcitabine into the bladder of the individual about once every 12 weeks (Q12W) for up to an additional 18 months after the first six months of treatment.

4. A method of treating Bacillus Calmette-Guérin (BCG)-naïve high-risk non-muscle invasive bladder cancer (HR-NMIBC) in an individual, comprising: inserting an intravesical drug delivery system comprising about 225 mg of gemcitabine into the bladder of the individual about once every three weeks (Q3W) for a total of up to six months following starting treatment, followed by inserting an intravesical drug delivery system comprising about 225 mg of gemcitabine into the bladder of the individual about once every 12 weeks (Q12W) for up to an additional 18 months after the first six months of treatment, wherein each intravesical drug delivery system is removed from the bladder about three weeks after insertion into the bladder and wherein each intravesical drug delivery system is removed prior to insertion of another intravesical drug delivery system.

5. The method of any one of embodiments 1-4, wherein inserting the intravesical drug delivery system into the bladder of the individual about once every three weeks (Q3W) for a total of up to six months following starting treatment comprises administering to the bladder of the individual a plurality of intravesical drug delivery systems, each intravesical drug delivery system comprising about 225 mg of gemcitabine, wherein administering the plurality of intravesical drug delivery systems comprises two or more three-week dosing cycles of gemcitabine, comprising: inserting one of the plurality of intravesical drug delivery systems into the bladder about once every three weeks (Q3W); and removing the intravesical drug delivery system from the bladder about three weeks after insertion, wherein in each dosing cycle of gemcitabine steps (i) and (ii) are repeated with a new one of the plurality of intravesical drug delivery systems for a total of up to six months following starting treatment.

6. The method of any one of embodiments 3-5, wherein inserting the intravesical drug delivery system into the bladder of the individual about once every 12 weeks (Q12W) for up to an additional 18 months after the first six months of treatment comprises administering to the bladder of the individual a plurality of intravesical drug delivery systems, each intravesical drug delivery system comprising about 225 mg of gemcitabine, wherein administering the plurality of intravesical drug delivery systems comprises twelve-week dosing cycles of gemcitabine comprising: inserting a new one of the plurality of intravesical drug delivery systems into the bladder about once every twelve weeks (Q12W); and removing the intravesical drug delivery system from the bladder about three weeks after insertion, wherein in each twelve-week dosing cycle of gemcitabine steps (iii) and (iv) are repeated with a new one of the plurality of intravesical drug delivery systems for up to the additional eighteen months after the first six months of treatment.

7. A method of treating Bacillus Calmette-Guérin (BCG)-naïve high-risk non-muscle invasive bladder cancer (HR-NMIBC) in an individual, comprising: intravenously administering to the individual about 360 mg of cetrelimab about once every three weeks (Q3W) for at least six months following starting treatment; and inserting an intravesical drug delivery system comprising about 225 mg of gemcitabine into the bladder of the individual about once every three weeks (Q3W) for a total of up to six months following starting treatment, wherein each intravesical drug delivery system is removed from the bladder about three weeks after insertion into the bladder and wherein each intravesical drug delivery system is removed prior to insertion of another intravesical drug delivery system.

8. A method of increasing event-free survival (EFS) for Bacillus Calmette-Guérin (BCG)-naïve high-risk non-muscle invasive bladder cancer (HR-NMIBC) in an individual, comprising: intravenously administering to the individual about 360 mg of cetrelimab about once every three weeks (Q3W) for at least six months following starting treatment; and inserting an intravesical drug delivery system comprising about 225 mg of gemcitabine into the bladder of the individual about once every three weeks (Q3W) for a total of up to six months following starting treatment, wherein each intravesical drug delivery system is removed from the bladder about three weeks after insertion into the bladder and wherein each intravesical drug delivery system is removed prior to insertion of another intravesical drug delivery system, and wherein the method results in an increased event-free survival (EFS) of the individual compared to the EFS achieved by intravesical BCG, and/or wherein the method results in an increased EFS in a population of patients receiving such therapy compared to a population of patients receiving intravesical BCG.

9. The method of embodiment 7 or 8, comprising inserting an intravesical drug delivery system comprising about 225 mg of gemcitabine into the bladder of the individual about once every 12 weeks (Q12W) for up to an additional 18 months after the first six months of treatment.

10. A method of treating Bacillus Calmette-Guérin (BCG)-naïve high-risk non-muscle invasive bladder cancer (HR-NMIBC) in an individual, comprising: intravenously administering to the individual about 360 mg of cetrelimab about once every three weeks (Q3W) for at least six months following starting treatment; and inserting an intravesical drug delivery system comprising about 225 mg of gemcitabine into the bladder of the individual about once every three weeks (Q3W) for a total of up to six months following starting treatment, followed by inserting an intravesical drug delivery system comprising about 225 mg of gemcitabine into the bladder of the individual about once every 12 weeks (Q12W) for up to an additional 18 months after the first six months of treatment, wherein each intravesical drug delivery system is removed from the bladder about three weeks after insertion into the bladder and wherein each intravesical drug delivery system is removed prior to insertion of another intravesical drug delivery system.

11. The method of embodiment 10, wherein intravenously administering to the individual about 360 mg of cetrelimab about once every three weeks (Q3W) for at least six months comprises intravenously administering to the individual about 360 mg of cetrelimab about Q3W for about one year.

12. The method of any one of embodiments 7-11, wherein inserting the intravesical drug delivery system into the bladder of the individual about once every three weeks (Q3W) for a total of up to six months following starting treatment comprises administering to the bladder of the individual a plurality of intravesical drug delivery systems, each intravesical drug delivery system comprising about 225 mg of gemcitabine, wherein administering the plurality of intravesical drug delivery systems comprises two or more three-week dosing cycles of gemcitabine, comprising: inserting one of the plurality of intravesical drug delivery systems into the bladder about once every three weeks (Q3W); and removing the intravesical drug delivery system from the bladder about three weeks after insertion, wherein in each dosing cycle of gemcitabine steps (i) and (ii) are repeated with a new one of the plurality of intravesical drug delivery systems for a total of up to six months following starting treatment.

13. The method of any one of embodiments 9-12, wherein inserting the intravesical drug delivery system into the bladder of the individual about once every 12 weeks (Q12W) for up to an additional 18 months after the first six months of treatment comprises administering to the bladder of the individual a plurality of intravesical drug delivery systems, each intravesical drug delivery system comprising about 225 mg of gemcitabine, wherein administering the plurality of intravesical drug delivery systems comprises twelve-week dosing cycles of gemcitabine comprising: inserting a new one of the plurality of intravesical drug delivery systems into the bladder about once every twelve weeks (Q12W); and removing the intravesical drug delivery system from the bladder about three weeks after insertion, wherein in each twelve-week dosing cycle of gemcitabine steps (iii) and (iv) are repeated with a new one of the plurality of intravesical drug delivery systems for up to the additional 18 months after the first six months of treatment.

14. A method of treating Bacillus Calmette-Guérin (BCG)-naïve high-risk non-muscle invasive bladder cancer (HR-NMIBC) in an individual, comprising: subcutaneously administering to the individual about 900 mg of cetrelimab at starting treatment, followed by subcutaneously administering to the individual about 600 mg of cetrelimab about once every three weeks (Q3W) for a total of up to six months following starting treatment; and inserting an intravesical drug delivery system comprising about 225 mg of gemcitabine into the bladder of the individual about once every three weeks (Q3W) for a total of up to six months following starting treatment, wherein each intravesical drug delivery system is removed from the bladder about three weeks after insertion into the bladder and wherein each intravesical drug delivery system is removed prior to insertion of another intravesical drug delivery system.

15. A method of increasing event-free survival (EFS) for Bacillus Calmette-Guérin (BCG)-naïve high-risk non-muscle invasive bladder cancer (HR-NMIBC) in an individual, comprising: subcutaneously administering to the individual about 900 mg of cetrelimab at starting treatment, followed by subcutaneously administering to the individual about 600 mg of cetrelimab about once every three weeks (Q3W) for at least 27 weeks following starting treatment; and inserting an intravesical drug delivery system comprising about 225 mg of gemcitabine into the bladder of the individual about once every three weeks (Q3W) for a total of up to six months following starting treatment, wherein each intravesical drug delivery system is removed from the bladder about three weeks after insertion into the bladder and wherein each intravesical drug delivery system is removed prior to insertion of another intravesical drug delivery system, and wherein the method results in an increased event-free survival (EFS) of the individual compared to the EFS achieved by intravesical BCG, and/or wherein the method results in an increased EFS in a population of patients receiving such therapy compared to a population of patients receiving intravesical BCG.

16. The method of embodiment 14 or 15, comprising inserting an intravesical drug delivery system comprising about 225 mg of gemcitabine into the bladder of the individual about once every 12 weeks (Q12W) for up to an additional 18 months after the first six months of treatment.

17. A method of treating Bacillus Calmette-Guérin (BCG)-naïve high-risk non-muscle invasive bladder cancer (HR-NMIBC) in an individual, comprising: subcutaneously administering to the individual about 900 mg of cetrelimab at starting treatment, followed by subcutaneously administering to the individual about 600 mg of cetrelimab about once every three weeks (Q3W) for a total of up to six months following starting treatment; and inserting an intravesical drug delivery system comprising about 225 mg of gemcitabine into the bladder of the individual about once every three weeks (Q3W) for a total of up to six months following starting treatment, followed by inserting an intravesical drug delivery system comprising about 225 mg of gemcitabine into the bladder of the individual about once every 12 weeks (Q12W) for up to an additional 18 months after the first six months of treatment, wherein each intravesical drug delivery system is removed from the bladder about three weeks after insertion into the bladder and wherein each intravesical drug delivery system is removed prior to insertion of another intravesical drug delivery system.

18. The method of any one of embodiments 14-17, wherein inserting the intravesical drug delivery system into the bladder of the individual about once every three weeks (Q3W) for a total of up to six months following starting treatment comprises administering to the bladder of the individual a plurality of intravesical drug delivery systems, each intravesical drug delivery system comprising about 225 mg of gemcitabine, wherein administering the plurality of intravesical drug delivery systems comprises two or more three-week dosing cycles of gemcitabine, comprising: inserting one of the plurality of intravesical drug delivery systems into the bladder about once every three weeks (Q3W); and removing the intravesical drug delivery system from the bladder about three weeks after insertion, wherein in each dosing cycle of gemcitabine steps (i) and (ii) are repeated with a new one of the plurality of intravesical drug delivery systems for a total of up to six months following starting treatment.

19. The method of any one of embodiments 16-18, wherein inserting the intravesical drug delivery system into the bladder of the individual about once every 12 weeks (Q12W) for up to an additional 18 months after the first six months of treatment comprises administering to the bladder of the individual a plurality of intravesical drug delivery systems, each intravesical drug delivery system comprising about 225 mg of gemcitabine, wherein administering the plurality of intravesical drug delivery systems comprises twelve-week dosing cycles of gemcitabine comprising: inserting a new one of the plurality of intravesical drug delivery systems into the bladder about once every twelve weeks (Q12W); and removing the intravesical drug delivery system from the bladder about three weeks after insertion, wherein in each twelve-week dosing cycle of gemcitabine steps (iii) and (iv) are repeated with a new one of the plurality of intravesical drug delivery systems for up to the additional 18 months after the first six months of treatment.

20. The method of any one of embodiments 1-19, wherein each intravesical drug delivery system comprises about 256 mg gemcitabine hydrochloride (HCl).

21. The method of any one of embodiments 7-20, wherein the administration of the cetrelimab and the insertion of the intravesical drug delivery system occurs no more than 72 hours apart from each other.

22. The method of any one of embodiments 7-20, wherein the administration of the cetrelimab and insertion of the intravesical drug delivery system occurs on the same day, and wherein the method comprises administering the intravesical drug delivery system at least 45 minutes prior to or after the completion of the administration of the cetrelimab.

23. The method of any one of embodiments 1-22, wherein inserting another intravesical drug delivery system occurs within a week of removing a previous intravesical drug delivery system within the first six months of treatment.

24. The method of any one of embodiments 3-6, 9-13, or 16-20, wherein, after the first year of treatment, inserting another intravesical drug delivery system occurs within 7-11 weeks of removing a previous intravesical drug delivery system.

25. The method of any one of embodiments 1-24, wherein inserting another intravesical drug delivery system occurs on the same day as removing a previous intravesical drug delivery system within the first six months of treatment.

26. The method of any one of embodiments 3-6, 9-13, 16-20, or 24, wherein inserting the intravesical drug delivery system into the bladder of the individual about once every 12 weeks (Q12W) for up to the additional 18 months begins at about week 36 after starting treatment, and wherein for a twelve-week period after the first six months of treatment, no intravesical drug delivery system is present in the bladder.

27. The method of any one of embodiments 1-26, wherein inserting the intravesical drug delivery system into the bladder of the individual about once every three weeks (Q3W) for a total of up to six months comprises eight three-week dosing cycles of gemcitabine.

28. The method of any one of embodiments 3-6, 9-13, 16-20, 24, or 26, wherein inserting the intravesical drug delivery system into the bladder of the individual about once every 12 weeks (Q12W) for up to an additional 18 months comprises up to six twelve-week dosing cycles of gemcitabine.

29. The method of any one of embodiments 1-28, wherein the method comprises up to fourteen total dosing cycles of gemcitabine.

30. The method of any one of embodiments 10-13, wherein intravenously administering the cetrelimab comprises eighteen dosing cycles of cetrelimab.

31. The method of any one of embodiments 17-19, wherein subcutaneously administering the cetrelimab comprises eight dosing cycles of cetrelimab.

32. The method of any one of embodiments 3-6, 9-13, 16-20, 24, 26, or 28, comprising, at about week 108 after start of treatment, inserting an intravesical drug delivery system comprising about 225 mg of gemcitabine into the bladder of the individual about once every 12 weeks (Q12W) for up to an additional ten months, wherein each intravesical drug delivery system is removed from the bladder about three weeks after insertion into the bladder and wherein each intravesical drug delivery system is removed prior to insertion of another intravesical drug delivery system.

33. The method of embodiment 32, wherein inserting an intravesical drug delivery system into the bladder of the individual about once every 12 weeks (Q12W) for up to an additional ten months comprises administering to the bladder of the individual a plurality of intravesical drug delivery systems, each intravesical drug delivery system comprising about 225 mg of gemcitabine, wherein administering the plurality of intravesical drug delivery systems comprises twelve-week dosing cycles of gemcitabine, comprising: inserting a new one of the plurality of intravesical drug delivery systems into the bladder about once every twelve weeks (Q12W); and removing the intravesical drug delivery system from the bladder about three weeks after insertion, wherein in each twelve-week dosing cycle of gemcitabine steps (v) and (vi) are repeated with a new one of the plurality of intravesical drug delivery systems for up to the additional ten months.

34. The method of embodiment 32 or 33, wherein inserting the intravesical drug delivery system into the bladder of the individual about once every 12 weeks (Q12W) for up to the additional ten months begins at about week 108 after start of treatment.

35. The method of any one of embodiments 32-34, wherein inserting the intravesical drug delivery system into the bladder of the individual about once every 12 weeks (Q12W) for up to the additional ten months comprises up to four twelve-week dosing cycles of gemcitabine.

36. The method of any one of embodiments 32-35, wherein the method comprises up to eighteen total dosing cycles of gemcitabine.

37. The method of any one of embodiments 1, 3-7, 9-14, or 16-36, wherein the method results in an increased event-free survival (EFS) compared to the EFS achieved by intravesical BCG, and/or wherein the method results in an increased EFS in a population of patients receiving such therapy compared to a population of patients receiving intravesical BCG.

38. The method of any one of embodiments 2, 8, 15, or 37, wherein an EFS event is defined by at least one of (a) positive local urine cytology, (b) positive local cystoscopy with biopsy proven high-risk disease, or (c) imaging proven disease progression or metastasis.

39. The method of any one of embodiments 1-38, comprising extending event-free survival (EFS) of the individual compared to the EFS achieved by intravesical BCG, and/or extending EFS in a population of patients receiving such therapy compared to a population of patients receiving intravesical BCG.

40. The method of any one of embodiments 1-39, wherein the method results in an increased overall complete response (CR) rate in a population of patients who have received treatment compared to the CR rate achieved by intravesical BCG.

41. The method of any one of embodiments 1-40, wherein the method results in an increased duration of CR compared to the duration of CR achieved by intravesical BCG.

42. The method of any one of embodiments 1-41, wherein the method results in an increased recurrence-free survival (RFS) compared to the RFS achieved by intravesical BCG.

43. The method of any one of embodiments 1-42, wherein the method results in an increased time to progression (TTP) compared to the TTP achieved by intravesical BCG.

44. The method of any one of embodiments 1-43, wherein the method results in an increased overall survival (OS) compared to the OS achieved by intravesical BCG.

45. The method of any one of embodiments 1-44, wherein the method results in an increased cancer specific survival (CSS) compared to the CSS achieved by intravesical BCG.

46. The method of any one of embodiments 1-45, wherein the method results in improved safety and tolerability compared to the safety and tolerability of intravesical BCG.

47. The method of any one of embodiments 1-46, wherein the method results in improved health-related quality of life (HRQOL) compared to the HRQOL of intravesical BCG.

48. The method of any one of embodiments 1-47, wherein the individual has high-grade Ta stage bladder cancer.

49. The method of any one of embodiments 1-48, wherein the individual has T1 stage bladder cancer.

50. The method of any one of embodiments 1-49, wherein the individual has carcinoma in situ (CIS).

51. The method of any one of embodiments 1-50, wherein the individual has a mixed histology tumor with predominant urothelial differentiation.

52. The method of any one of embodiments 1-51, wherein the individual has an Eastern Cooperative Oncology Group (ECOG) performance status Grade 0, 1, or 2.

53. The method of any one of embodiments 1-52, wherein the individual has papillary disease, and wherein the papillary disease is fully resected prior to treatment with gemcitabine.

54. The method of any one of embodiments 1-53, wherein the individual does not have a presence or history of histologically confirmed, muscle invasive, locally advanced, nonresectable, or metastatic urothelial carcinoma of stage T2 or greater.

55. The method of any one of embodiments 1-54, wherein the individual does not have urothelial carcinoma or a histological variant at any site outside of the bladder.

56. The method of any one of embodiments 1-55, wherein the individual does not have a history of HR-NMIBC less than three years from current diagnosis.

57. The method of any one of embodiments 1-56, wherein the individual does not have visible papillary disease at the time of diagnosis.

58. The method of any one of embodiments 1-57, wherein the individual does not have N+ and/or M+ bladder cancer.

59. The method of any one of embodiments 1-58, wherein the individual does not have neuroendocrine, micropapillary, signet ring cell, plasmacytoid, or sarcomatoid features.

60. The method of any one of embodiments 1-59, wherein the individual does not have a presence of any bladder or urothelial anatomical feature that prevents the safe insertion, indwelling use, and removal of the intravesical drug delivery system.

61. The method of any one of embodiments 1-60, wherein the individual does not have a history of clinically significant polyuria with recorded 24-hour urine volumes greater than 4000 mL.

62. The method of any one of embodiments 1-61, wherein the individual has not received BCG within three years from diagnosis.

63. The method of any one of embodiments 1-62, wherein the individual is instructed to drink at least 1500 mL of fluid per day during each dosing cycle.

64. The method of any one of embodiments 1-63, comprising administering anticholinergics, nonsteroidal anti-inflammatory drugs (NSAIDs), or bladder analgesics such as phenazopyridine prior to or after insertion or removal of the one of the plurality of intravesical drug delivery systems.

65. The method of any one of embodiments 1-64, comprising administering prophylactic antibiotics with insertion and removal of the intravesical drug delivery system.

66. The method of any one of embodiments 1-65, wherein the intravesical drug delivery system is inserted into the bladder transurethrally using a catheter.

67. The method of any one of embodiments 1-66, wherein the intravesical drug delivery system is removed using forceps and a cystoscope.

68. The method of any one of embodiments 1-67, wherein the intravesical drug delivery system comprises a flexible, bi-oval shaped housing comprising a dual lumen.

69. The method of embodiment 68, wherein the dual lumen comprises a drug reservoir lumen containing the gemcitabine and a retention frame lumen containing a predefined shaped retention frame.

70. The method of embodiment 69, wherein the retention frame is a super-elastic nitinol wire.

71. The method of embodiment 69 or 70, wherein the drug reservoir lumen comprises a single delivery orifice that controllably releases the gemcitabine from the drug reservoir lumen through the delivery orifice.

72. The method of embodiment 71, wherein the delivery orifice is a laser-drilled delivery orifice.

73. The method of any one of embodiments 69-72, wherein the intravesical drug delivery system comprises a first unit contained within the drug reservoir lumen and comprising the gemcitabine and a second unit contained within the drug reservoir lumen in a position distinct from the first unit, the second unit comprising an osmotic agent.

74. The method of embodiment 73, wherein the osmotic agent comprises urea minitablets.

75. The method of embodiment 73 or 74, wherein the first unit further comprises urea.

76. The method of any one of embodiments 73-75, wherein the first unit contains at least 75 percent by weight gemcitabine HCL.

77. The method of any one of embodiments 73-76, wherein the second unit contains at least 85 percent by weight urea.

78. The method of any one of embodiments 71-77, wherein the housing is configured to release the gemcitabine from the drug reservoir lumen through the delivery orifice via osmotic pressure generated by the osmotic agent.

79. The method of any one of embodiments 68-78, wherein the housing is elastically deformable between a retention shape configured to retain the intravesical drug delivery system in the individual's bladder and a deployment shape configured for passage of the intravesical drug delivery system through the individual's urethra.

80. The method of any one of embodiments 68-79, wherein the gemcitabine is in a non-liquid form.

81. The method of embodiment 80, wherein the gemcitabine is in the form of minitablets.

82. The method of any one of embodiments 1-81, wherein the intravesical drug delivery system releases gemcitabine at a zero-order release rate for an extended period, followed by a reduced, non-zero-order release rate over a decay period.

83. The method of any one of embodiments 1-82, wherein the concentration of gemcitabine in the urine of the individual is between about 4 μg/mL and about 50 μg/mL while the intravesical drug delivery system is inserted into the bladder.

84. The method of any one of embodiments 1-83, wherein about 35% to about 55% of the gemcitabine in the intravesical drug delivery system is released into the urine of the individual by day seven after insertion into the bladder.

85. The method of any one of embodiments 1-84, wherein about 70% to about 90% of the gemcitabine in the intravesical drug delivery system is released into the urine of the individual by day 21 after insertion into the bladder.

86. The method of any of embodiments 1-85, wherein inserting the intravesical drug delivery system into the bladder of the individual about once every three weeks (Q3W) for a total of up to six months following starting treatment comprises the following steps: inserting a first intravesical drug delivery system of the plurality of intravesical drug delivery systems into the bladder at about week 0; removing the first intravesical drug delivery system at about week 3; inserting a second intravesical drug delivery system of the plurality of intravesical drug delivery systems into the bladder at about week 3; removing the second intravesical drug delivery system at about week 6; inserting a third intravesical drug delivery system of the plurality of intravesical drug delivery systems into the bladder at about week 6; removing the third intravesical drug delivery system at about week 9; inserting a fourth intravesical drug delivery system of the plurality of intravesical drug delivery systems into the bladder at about week 9; removing the fourth intravesical drug delivery system at about week 12; inserting a fifth intravesical drug delivery system of the plurality of intravesical drug delivery systems into the bladder at about week 12; removing the fifth intravesical drug delivery system at about week 15; inserting a sixth intravesical drug delivery system of the plurality of intravesical drug delivery systems into the bladder at about week 15; removing the sixth intravesical drug delivery system at about week 18; inserting a seventh intravesical drug delivery system of the plurality of intravesical drug delivery systems into the bladder at about week 18; removing the seventh intravesical drug delivery system at about week 21; inserting an eighth intravesical drug delivery system of the plurality of intravesical drug delivery systems into the bladder at about week 21; and removing the eighth intravesical drug delivery system at about week 24, wherein each intravesical drug delivery system comprises about 225 mg of gemcitabine.

87. The method of any one of embodiments 3-6, 9-13, 16-20, 24, 26, 28, 32-36, or 86, wherein inserting the intravesical drug delivery system into the bladder of the individual about once every 12 weeks (Q12W) for up to the additional 18 months after the first six months of treatment comprises the following steps: inserting a ninth intravesical drug delivery system of the plurality of intravesical drug delivery systems into the bladder at about week 36; removing the ninth intravesical drug delivery system at about week 39; inserting a tenth intravesical drug delivery system of the plurality of intravesical drug delivery systems into the bladder at about week 48; removing the tenth intravesical drug delivery system at about week 51; inserting an eleventh intravesical drug delivery system of the plurality of intravesical drug delivery systems into the bladder at about week 60; removing the eleventh intravesical drug delivery system at about week 63; inserting a twelfth intravesical drug delivery system of the plurality of intravesical drug delivery systems into the bladder at about week 72; removing the twelfth intravesical drug delivery system at about week 75; inserting a thirteenth intravesical drug delivery system of the plurality of intravesical drug delivery systems into the bladder at about week 84; removing the thirteenth intravesical drug delivery system at about week 87; inserting a fourteenth intravesical drug delivery system of the plurality of intravesical drug delivery systems into the bladder at about week 96; and removing the fourteenth intravesical drug delivery system at about week 99, wherein each intravesical drug delivery system comprises about 225 mg of gemcitabine.

88. The method of any one of embodiments 1-87, wherein the total of up to six months ends with removal of an intravesical drug delivery system at about six months.

89. The method of any one of embodiments 3-6, 9-13, 16-20, 24, 26, 28, 32-36, or 87, wherein the additional 18 months after the first six months of treatment ends with removal of an intravesical drug delivery system at about 18 months following starting treatment.

90. The method of embodiment 86 or 87, wherein each intravesical drug delivery systems comprises about 256 mg of gemcitabine HCL.

91. A method of treating Bacillus Calmette-Guérin (BCG)-naïve high-risk non-muscle invasive bladder cancer (HR-NMIBC) in an individual, comprising inserting an intravesical drug delivery system comprising about 225 mg of gemcitabine into the bladder once every 3 weeks up to 6 months (8 doses), followed by once every 12 weeks for up to 2 years from first dose, or until persistent or recurrent HR-NMIBC, disease progression, or unacceptable toxicity.

92. The method of embodiment 91, wherein the intravesical drug delivery system is administered as a monotherapy for treating Bacillus Calmette-Guérin (BCG)-naïve high-risk non-muscle invasive bladder cancer (HR-NMIBC).

93. A method of treating Bacillus Calmette-Guérin (BCG)-naïve high-risk non-muscle invasive bladder cancer (HR-NMIBC) in an individual, comprising: inserting an intravesical drug delivery system comprising about 225 mg of gemcitabine into the bladder once every 3 weeks up to 6 months (8 doses), followed by once every 12 weeks for up to 2 years from first dose, or until persistent or recurrent HR-NMIBC, disease progression, or unacceptable toxicity, and administering cetrelimab for up to 1 year from first dose, or until persistent or recurrent HR-NMIBC, disease progression, or unacceptable toxicity.

94. A method of treating Bacillus Calmette-Guérin (BCG)-naïve non-muscle invasive bladder cancer (NMIBC) in an individual, comprising inserting an intravesical drug delivery system comprising about 225 mg of gemcitabine into the bladder once every 3 weeks up to 6 months (8 doses), followed by once every 12 weeks for up to 2 years from first dose, or until persistent or recurrent NMIBC, disease progression, or unacceptable toxicity.

95. The method of embodiment 94, wherein the intravesical drug delivery system is administered as a monotherapy for treating Bacillus Calmette-Guérin (BCG)-naïve non-muscle invasive bladder cancer (NMIBC).

96. A method of treating Bacillus Calmette-Guérin (BCG)-naïve non-muscle invasive bladder cancer (NMIBC) in an individual, comprising: inserting an intravesical drug delivery system comprising about 225 mg of gemcitabine into the bladder once every 3 weeks up to 6 months (8 doses), followed by once every 12 weeks for up to 2 years from first dose, or until persistent or recurrent NMIBC, disease progression, or unacceptable toxicity, and administering cetrelimab for up to 1 year from first dose, or until persistent or recurrent NMIBC, disease progression, or unacceptable toxicity.

97. The method of any one of embodiments 91-96, wherein each intravesical system is removed after a 3-week indwelling period.

98. A method of treating Bacillus Calmette-Guérin (BCG)-naïve non-muscle invasive bladder cancer (NMIBC) in an individual, comprising: inserting an intravesical drug delivery system comprising about 225 mg of gemcitabine into the bladder of the individual about once every three weeks (Q3W) for a total of up to six months following starting treatment, wherein each intravesical drug delivery system is removed from the bladder about three weeks after insertion into the bladder and wherein each intravesical drug delivery system is removed prior to insertion of another intravesical drug delivery system.

99. A method of increasing event-free survival (EFS) for Bacillus Calmette-Guérin (BCG)-naïve non-muscle invasive bladder cancer (NMIBC) in an individual, comprising: inserting an intravesical drug delivery system comprising about 225 mg of gemcitabine into the bladder of the individual about once every three weeks (Q3W) for a total of up to six months following starting treatment, wherein each intravesical drug delivery system is removed from the bladder about three weeks after insertion into the bladder and wherein each intravesical drug delivery system is removed prior to insertion of another intravesical drug delivery system, and wherein the method results in an increased event-free survival (EFS) of the individual compared to the EFS achieved by intravesical BCG, and/or wherein the method results in an increased EFS in a population of patients receiving such therapy compared to a population of patients receiving intravesical BCG.

100. The method of embodiment 98 or 99, comprising inserting an intravesical drug delivery system comprising about 225 mg of gemcitabine into the bladder of the individual about once every 12 weeks (Q12W) for up to an additional 18 months after the first six months of treatment.

101. A method of treating Bacillus Calmette-Guérin (BCG)-naïve non-muscle invasive bladder cancer (NMIBC) in an individual, comprising: inserting an intravesical drug delivery system comprising about 225 mg of gemcitabine into the bladder of the individual about once every three weeks (Q3W) for a total of up to six months following starting treatment, followed by inserting an intravesical drug delivery system comprising about 225 mg of gemcitabine into the bladder of the individual about once every 12 weeks (Q12W) for up to an additional 18 months after the first six months of treatment, wherein each intravesical drug delivery system is removed from the bladder about three weeks after insertion into the bladder and wherein each intravesical drug delivery system is removed prior to insertion of another intravesical drug delivery system.

102. The method of any one of embodiments 98-101, wherein inserting the intravesical drug delivery system into the bladder of the individual about once every three weeks (Q3W) for a total of up to six months following starting treatment comprises administering to the bladder of the individual a plurality of intravesical drug delivery systems, each intravesical drug delivery system comprising about 225 mg of gemcitabine, wherein administering the plurality of intravesical drug delivery systems comprises two or more three-week dosing cycles of gemcitabine, comprising: (i) inserting one of the plurality of intravesical drug delivery systems into the bladder about once every three weeks (Q3W); and (ii) removing the intravesical drug delivery system from the bladder about three weeks after insertion, wherein in each dosing cycle of gemcitabine steps (i) and (ii) are repeated with a new one of the plurality of intravesical drug delivery systems for a total of up to six months following starting treatment.

103. The method of any one of embodiments 100-102, wherein inserting the intravesical drug delivery system into the bladder of the individual about once every 12 weeks (Q12W) for up to an additional 18 months after the first six months of treatment comprises administering to the bladder of the individual a plurality of intravesical drug delivery systems, each intravesical drug delivery system comprising about 225 mg of gemcitabine, wherein administering the plurality of intravesical drug delivery systems comprises twelve-week dosing cycles of gemcitabine comprising: (iii) inserting a new one of the plurality of intravesical drug delivery systems into the bladder about once every twelve weeks (Q12W); and (iv) removing the intravesical drug delivery system from the bladder about three weeks after insertion, wherein in each twelve-week dosing cycle of gemcitabine steps (iii) and (iv) are repeated with a new one of the plurality of intravesical drug delivery systems for up to the additional eighteen months after the first six months of treatment.

104. A method of treating Bacillus Calmette-Guérin (BCG)-naïve non-muscle invasive bladder cancer (NMIBC) in an individual, comprising: intravenously administering to the individual about 360 mg of cetrelimab about once every three weeks (Q3W) for at least six months following starting treatment; and inserting an intravesical drug delivery system comprising about 225 mg of gemcitabine into the bladder of the individual about once every three weeks (Q3W) for a total of up to six months following starting treatment, wherein each intravesical drug delivery system is removed from the bladder about three weeks after insertion into the bladder and wherein each intravesical drug delivery system is removed prior to insertion of another intravesical drug delivery system.

105. A method of increasing event-free survival (EFS) for Bacillus Calmette-Guérin (BCG)-naïve non-muscle invasive bladder cancer (NMIBC) in an individual, comprising: intravenously administering to the individual about 360 mg of cetrelimab about once every three weeks (Q3W) for at least six months following starting treatment; and inserting an intravesical drug delivery system comprising about 225 mg of gemcitabine into the bladder of the individual about once every three weeks (Q3W) for a total of up to six months following starting treatment, wherein each intravesical drug delivery system is removed from the bladder about three weeks after insertion into the bladder and wherein each intravesical drug delivery system is removed prior to insertion of another intravesical drug delivery system, and wherein the method results in an increased event-free survival (EFS) of the individual compared to the EFS achieved by intravesical BCG, and/or wherein the method results in an increased EFS in a population of patients receiving such therapy compared to a population of patients receiving intravesical BCG.

106. The method of embodiment 104 or 105, comprising inserting an intravesical drug delivery system comprising about 225 mg of gemcitabine into the bladder of the individual about once every 12 weeks (Q12W) for up to an additional 18 months after the first six months of treatment.

107. A method of treating Bacillus Calmette-Guérin (BCG)-naïve non-muscle invasive bladder cancer (NMIBC) in an individual, comprising: intravenously administering to the individual about 360 mg of cetrelimab about once every three weeks (Q3W) for at least six months following starting treatment; and inserting an intravesical drug delivery system comprising about 225 mg of gemcitabine into the bladder of the individual about once every three weeks (Q3W) for a total of up to six months following starting treatment, followed by inserting an intravesical drug delivery system comprising about 225 mg of gemcitabine into the bladder of the individual about once every 12 weeks (Q12W) for up to an additional 18 months after the first six months of treatment, wherein each intravesical drug delivery system is removed from the bladder about three weeks after insertion into the bladder and wherein each intravesical drug delivery system is removed prior to insertion of another intravesical drug delivery system.

108. The method of embodiment 107, wherein intravenously administering to the individual about 360 mg of cetrelimab about once every three weeks (Q3W) for at least six months comprises intravenously administering to the individual about 360 mg of cetrelimab about Q3W for about one year.

109. The method of any one of embodiments 104-108, wherein inserting the intravesical drug delivery system into the bladder of the individual about once every three weeks (Q3W) for a total of up to six months following starting treatment comprises administering to the bladder of the individual a plurality of intravesical drug delivery systems, each intravesical drug delivery system comprising about 225 mg of gemcitabine, wherein administering the plurality of intravesical drug delivery systems comprises two or more three-week dosing cycles of gemcitabine, comprising: (i) inserting one of the plurality of intravesical drug delivery systems into the bladder about once every three weeks (Q3W); and (ii) removing the intravesical drug delivery system from the bladder about three weeks after insertion, wherein in each dosing cycle of gemcitabine steps (i) and (ii) are repeated with a new one of the plurality of intravesical drug delivery systems for a total of up to six months following starting treatment.

110. The method of any one of embodiments 106-109, wherein inserting the intravesical drug delivery system into the bladder of the individual about once every 12 weeks (Q12W) for up to an additional 18 months after the first six months of treatment comprises administering to the bladder of the individual a plurality of intravesical drug delivery systems, each intravesical drug delivery system comprising about 225 mg of gemcitabine, wherein administering the plurality of intravesical drug delivery systems comprises twelve-week dosing cycles of gemcitabine comprising: (iii) inserting a new one of the plurality of intravesical drug delivery systems into the bladder about once every twelve weeks (Q12W); and (iv) removing the intravesical drug delivery system from the bladder about three weeks after insertion, wherein in each twelve-week dosing cycle of gemcitabine steps (iii) and (iv) are repeated with a new one of the plurality of intravesical drug delivery systems for up to the additional 18 months after the first six months of treatment.

111. A method of treating Bacillus Calmette-Guérin (BCG)-naïve non-muscle invasive bladder cancer (NMIBC) in an individual, comprising: subcutaneously administering to the individual about 900 mg of cetrelimab at starting treatment, followed by subcutaneously administering to the individual about 600 mg of cetrelimab about once every three weeks (Q3W) for a total of up to six months following starting treatment; and inserting an intravesical drug delivery system comprising about 225 mg of gemcitabine into the bladder of the individual about once every three weeks (Q3W) for a total of up to six months following starting treatment, wherein each intravesical drug delivery system is removed from the bladder about three weeks after insertion into the bladder and wherein each intravesical drug delivery system is removed prior to insertion of another intravesical drug delivery system.

112. A method of increasing event-free survival (EFS) for Bacillus Calmette-Guérin (BCG)-naïve non-muscle invasive bladder cancer (NMIBC) in an individual, comprising: subcutaneously administering to the individual about 900 mg of cetrelimab at starting treatment, followed by subcutaneously administering to the individual about 600 mg of cetrelimab about once every three weeks (Q3W) for at least 27 weeks following starting treatment; and inserting an intravesical drug delivery system comprising about 225 mg of gemcitabine into the bladder of the individual about once every three weeks (Q3W) for a total of up to six months following starting treatment, wherein each intravesical drug delivery system is removed from the bladder about three weeks after insertion into the bladder and wherein each intravesical drug delivery system is removed prior to insertion of another intravesical drug delivery system, and wherein the method results in an increased event-free survival (EFS) of the individual compared to the EFS achieved by intravesical BCG, and/or wherein the method results in an increased EFS in a population of patients receiving such therapy compared to a population of patients receiving intravesical BCG.

113. The method of embodiment 111 or 112, comprising inserting an intravesical drug delivery system comprising about 225 mg of gemcitabine into the bladder of the individual about once every 12 weeks (Q12W) for up to an additional 18 months after the first six months of treatment.

114. A method of treating Bacillus Calmette-Guérin (BCG)-naïve non-muscle invasive bladder cancer (NMIBC) in an individual, comprising: subcutaneously administering to the individual about 900 mg of cetrelimab at starting treatment, followed by subcutaneously administering to the individual about 600 mg of cetrelimab about once every three weeks (Q3W) for a total of up to six months following starting treatment; and inserting an intravesical drug delivery system comprising about 225 mg of gemcitabine into the bladder of the individual about once every three weeks (Q3W) for a total of up to six months following starting treatment, followed by inserting an intravesical drug delivery system comprising about 225 mg of gemcitabine into the bladder of the individual about once every 12 weeks (Q12W) for up to an additional 18 months after the first six months of treatment, wherein each intravesical drug delivery system is removed from the bladder about three weeks after insertion into the bladder and wherein each intravesical drug delivery system is removed prior to insertion of another intravesical drug delivery system.

115. The method of any one of embodiments 111-114, wherein inserting the intravesical drug delivery system into the bladder of the individual about once every three weeks (Q3W) for a total of up to six months following starting treatment comprises administering to the bladder of the individual a plurality of intravesical drug delivery systems, each intravesical drug delivery system comprising about 225 mg of gemcitabine, wherein administering the plurality of intravesical drug delivery systems comprises two or more three-week dosing cycles of gemcitabine, comprising: (v) inserting one of the plurality of intravesical drug delivery systems into the bladder about once every three weeks (Q3W); and (vi) removing the intravesical drug delivery system from the bladder about three weeks after insertion, wherein in each dosing cycle of gemcitabine steps (i) and (ii) are repeated with a new one of the plurality of intravesical drug delivery systems for a total of up to six months following starting treatment.

116. The method of any one of embodiments 113-115, wherein inserting the intravesical drug delivery system into the bladder of the individual about once every 12 weeks (Q12W) for up to an additional 18 months after the first six months of treatment comprises administering to the bladder of the individual a plurality of intravesical drug delivery systems, each intravesical drug delivery system comprising about 225 mg of gemcitabine, wherein administering the plurality of intravesical drug delivery systems comprises twelve-week dosing cycles of gemcitabine comprising: (vii) inserting a new one of the plurality of intravesical drug delivery systems into the bladder about once every twelve weeks (Q12W); and (viii) removing the intravesical drug delivery system from the bladder about three weeks after insertion, wherein in each twelve-week dosing cycle of gemcitabine steps (iii) and (iv) are repeated with a new one of the plurality of intravesical drug delivery systems for up to the additional 18 months after the first six months of treatment.

117. The method of any one of embodiments 94-116, wherein each intravesical drug delivery system comprises about 256 mg gemcitabine hydrochloride (HCl).

118. The method of any one of embodiments 96 or 104-117, wherein the administration of the cetrelimab and the insertion of the intravesical drug delivery system occurs no more than 72 hours apart from each other.

119. The method of any one of embodiments 96 or 104-117, wherein the administration of the cetrelimab and insertion of the intravesical drug delivery system occurs on the same day, and wherein the method comprises administering the intravesical drug delivery system at least 45 minutes prior to or after the completion of the administration of the cetrelimab.

120. The method of any one of embodiments 94-119, wherein inserting another intravesical drug delivery system occurs within a week of removing a previous intravesical drug delivery system within the first six months of treatment.

121. The method of any one of embodiments 100-103, 106-110, or 113-117, wherein, after the first year of treatment, inserting another intravesical drug delivery system occurs within 7-11 weeks of removing a previous intravesical drug delivery system.

122. The method of any one of embodiments 94-121, wherein inserting another intravesical drug delivery system occurs on the same day as removing a previous intravesical drug delivery system within the first six months of treatment.

123. The method of any one of embodiments 100-103, 106-110, 113-117, or 121, wherein inserting the intravesical drug delivery system into the bladder of the individual about once every 12 weeks (Q12W) for up to the additional 18 months begins at about week 36 after starting treatment, and wherein for a twelve-week period after the first six months of treatment, no intravesical drug delivery system is present in the bladder.

124. The method of any one of embodiments 94-123, wherein inserting the intravesical drug delivery system into the bladder of the individual about once every three weeks (Q3W) for a total of up to six months comprises eight three-week dosing cycles of gemcitabine.

125. The method of any one of embodiments 100-103, 106-110, 113-117, 121, or 123, wherein inserting the intravesical drug delivery system into the bladder of the individual about once every 12 weeks (Q12W) for up to an additional 18 months comprises up to six twelve-week dosing cycles of gemcitabine.

126. The method of any one of embodiments 94-125, wherein the method comprises up to fourteen total dosing cycles of gemcitabine.

127. The method of any one of embodiments 96 or 107-110, wherein intravenously administering the cetrelimab comprises eighteen dosing cycles of cetrelimab.

128. The method of any one of embodiments 96 or 114-117, wherein subcutaneously administering the cetrelimab comprises eight dosing cycles of cetrelimab.

129. The method of any one of embodiments 100-103, 106-110, 113-117, 121, 123, or 125, comprising, at about week 108 after start of treatment, inserting an intravesical drug delivery system comprising about 225 mg of gemcitabine into the bladder of the individual about once every 12 weeks (Q12W) for up to an additional ten months, wherein each intravesical drug delivery system is removed from the bladder about three weeks after insertion into the bladder and wherein each intravesical drug delivery system is removed prior to insertion of another intravesical drug delivery system.

130. The method of embodiment 129, wherein inserting an intravesical drug delivery system into the bladder of the individual about once every 12 weeks (Q12W) for up to an additional ten months comprises administering to the bladder of the individual a plurality of intravesical drug delivery systems, each intravesical drug delivery system comprising about 225 mg of gemcitabine, wherein administering the plurality of intravesical drug delivery systems comprises twelve-week dosing cycles of gemcitabine, comprising: (v) inserting a new one of the plurality of intravesical drug delivery systems into the bladder about once every twelve weeks (Q12W); and (vi) removing the intravesical drug delivery system from the bladder about three weeks after insertion, wherein in each twelve-week dosing cycle of gemcitabine steps (v) and (vi) are repeated with a new one of the plurality of intravesical drug delivery systems for up to the additional ten months.

131. The method of embodiment 129 or 130, wherein inserting the intravesical drug delivery system into the bladder of the individual about once every 12 weeks (Q12W) for up to the additional ten months begins at about week 108 after start of treatment.

132. The method of any one of embodiments 129-131, wherein inserting the intravesical drug delivery system into the bladder of the individual about once every 12 weeks (Q12W) for up to the additional ten months comprises up to four twelve-week dosing cycles of gemcitabine.

133. The method of any one of embodiments 129-132, wherein the method comprises up to eighteen total dosing cycles of gemcitabine.

134. The method of any one of embodiments 94-133, wherein the method results in an increased event-free survival (EFS) compared to the EFS achieved by intravesical BCG, and/or wherein the method results in an increased EFS in a population of patients receiving such therapy compared to a population of patients receiving intravesical BCG.

135. The method of any one of embodiments 134, wherein an EFS event is defined by at least one of (a) positive local urine cytology, (b) positive local cystoscopy with biopsy proven high-risk disease, or (c) imaging proven disease progression or metastasis.

136. The method of any one of embodiments 94-135, comprising extending event-free survival (EFS) of the individual compared to the EFS achieved by intravesical BCG, and/or extending EFS in a population of patients receiving such therapy compared to a population of patients receiving intravesical BCG.

137. The method of any one of embodiments 94-136, wherein the method results in an increased overall complete response (CR) rate in a population of patients who have received treatment compared to the CR rate achieved by intravesical BCG.

138. The method of any one of embodiments 94-137, wherein the method results in an increased duration of CR compared to the duration of CR achieved by intravesical BCG.

139. The method of any one of embodiments 94-138, wherein the method results in an increased recurrence-free survival (RFS) compared to the RFS achieved by intravesical BCG.

140. The method of any one of embodiments 94-139, wherein the method results in an increased time to progression (TTP) compared to the TTP achieved by intravesical BCG.

141. The method of any one of embodiments 94-140, wherein the method results in an increased overall survival (OS) compared to the OS achieved by intravesical BCG.

142. The method of any one of embodiments 94-141, wherein the method results in an increased cancer specific survival (CSS) compared to the CSS achieved by intravesical BCG.

143. The method of any one of embodiments 94-142, wherein the method results in improved safety and tolerability compared to the safety and tolerability of intravesical BCG.

144. The method of any one of embodiments 94-143, wherein the method results in improved health-related quality of life (HRQOL) compared to the HRQOL of intravesical BCG.

145. The method of any one of embodiments 94-144, wherein the individual has high-grade Ta stage bladder cancer.

146. The method of any one of embodiments 94-145, wherein the individual has T1 stage bladder cancer.

147. The method of any one of embodiments 94-146, wherein the individual has carcinoma in situ (CIS).

148. The method of any one of embodiments 94-147, wherein the individual has a mixed histology tumor with predominant urothelial differentiation.

149. The method of any one of embodiments 94-148, wherein the individual has an Eastern Cooperative Oncology Group (ECOG) performance status Grade 0, 1, or 2.

150. The method of any one of embodiments 94-149, wherein the individual has papillary disease, and wherein the papillary disease is fully resected prior to treatment with gemcitabine.

151. The method of any one of embodiments 94-150, wherein the individual does not have a presence or history of histologically confirmed, muscle invasive, locally advanced, nonresectable, or metastatic urothelial carcinoma of stage T2 or greater.

152. The method of any one of embodiments 94-151, wherein the individual does not have urothelial carcinoma or a histological variant at any site outside of the bladder.

153. The method of any one of embodiments 94-152, wherein the individual does not have a history of NMIBC less than three years from current diagnosis.

154. The method of any one of embodiments 94-153, wherein the individual does not have visible papillary disease at the time of diagnosis.

155. The method of any one of embodiments 94-154, wherein the individual does not have N+ and/or M+ bladder cancer.

156. The method of any one of embodiments 94-155, wherein the individual does not have neuroendocrine, micropapillary, signet ring cell, plasmacytoid, or sarcomatoid features.

157. The method of any one of embodiments 94-156, wherein the individual does not have a presence of any bladder or urothelial anatomical feature that prevents the safe insertion, indwelling use, and removal of the intravesical drug delivery system.

158. The method of any one of embodiments 94-157, wherein the individual does not have a history of clinically significant polyuria with recorded 24-hour urine volumes greater than 4000 mL.

159. The method of any one of embodiments 94-158, wherein the individual has not received BCG within three years from diagnosis.

160. The method of any one of embodiments 94-159, wherein the individual is instructed to drink at least 1500 mL of fluid per day during each dosing cycle.

161. The method of any one of embodiments 94-160, comprising administering anticholinergics, nonsteroidal anti-inflammatory drugs (NSAIDs), or bladder analgesics such as phenazopyridine prior to or after insertion or removal of the one of the plurality of intravesical drug delivery systems.

162. The method of any one of embodiments 94-161, comprising administering prophylactic antibiotics with insertion and removal of the intravesical drug delivery system.

163. The method of any one of embodiments 94-162, wherein the intravesical drug delivery system is inserted into the bladder transurethrally using a catheter.

164. The method of any one of embodiments 94-163, wherein the intravesical drug delivery system is removed using forceps and a cystoscope.

165. The method of any one of embodiments 94-164, wherein the intravesical drug delivery system comprises a flexible, bi-oval shaped housing comprising a dual lumen.

166. The method of embodiment 165, wherein the dual lumen comprises a drug reservoir lumen containing the gemcitabine and a retention frame lumen containing a predefined shaped retention frame.

167. The method of embodiment 166, wherein the retention frame is a super-elastic nitinol wire.

168. The method of embodiment 165-166, wherein the drug reservoir lumen comprises a single delivery orifice that controllably releases the gemcitabine from the drug reservoir lumen through the delivery orifice.

169. The method of embodiment 168, wherein the delivery orifice is a laser-drilled delivery orifice.

170. The method of any one of embodiments 166-169, wherein the intravesical drug delivery system comprises a first unit contained within the drug reservoir lumen and comprising the gemcitabine and a second unit contained within the drug reservoir lumen in a position distinct from the first unit, the second unit comprising an osmotic agent.

171. The method of embodiment 170, wherein the osmotic agent comprises urea minitablets.

172. The method of embodiment 170 or 171, wherein the first unit further comprises urea.

173. The method of any one of embodiments 170-172, wherein the first unit contains at least 75 percent by weight gemcitabine HCL.

174. The method of any one of embodiments 170-173, wherein the second unit contains at least 85 percent by weight urea.

175. The method of any one of embodiments 168-174, wherein the housing is configured to release the gemcitabine from the drug reservoir lumen through the delivery orifice via osmotic pressure generated by the osmotic agent.

176. The method of any one of embodiments 165-175, wherein the housing is elastically deformable between a retention shape configured to retain the intravesical drug delivery system in the individual's bladder and a deployment shape configured for passage of the intravesical drug delivery system through the individual's urethra.

177. The method of any one of embodiments 165-176, wherein the gemcitabine is in a non-liquid form.

178. The method of embodiment 177, wherein the gemcitabine is in the form of minitablets.

179. The method of any one of embodiments 94-178, wherein the intravesical drug delivery system releases gemcitabine at a zero-order release rate for an extended period, followed by a reduced, non-zero-order release rate over a decay period.

180. The method of any one of embodiments 94-179, wherein the concentration of gemcitabine in the urine of the individual is between about 4 μg/mL and about 50 μg/mL while the intravesical drug delivery system is inserted into the bladder.

181. The method of any one of embodiments 94-180, wherein about 65% to about 85% of the gemcitabine in the intravesical drug delivery system is released into the urine of the individual by day seven after insertion into the bladder.

182. The method of any one of embodiments 94-181, wherein about 85% to about 100% of the gemcitabine in the intravesical drug delivery system is released into the urine of the individual by day 21 after insertion into the bladder.

183. The method of any of embodiments 94-182, wherein inserting the intravesical drug delivery system into the bladder of the individual about once every three weeks (Q3W) for a total of up to six months following starting treatment comprises the following steps: (i) inserting a first intravesical drug delivery system of the plurality of intravesical drug delivery systems into the bladder at about week 0; (ii) removing the first intravesical drug delivery system at about week 3; (iii) inserting a second intravesical drug delivery system of the plurality of intravesical drug delivery systems into the bladder at about week 3; (iv) removing the second intravesical drug delivery system at about week 6; (v) inserting a third intravesical drug delivery system of the plurality of intravesical drug delivery systems into the bladder at about week 6; (vi) removing the third intravesical drug delivery system at about week 9; (vii) inserting a fourth intravesical drug delivery system of the plurality of intravesical drug delivery systems into the bladder at about week 9; (viii) removing the fourth intravesical drug delivery system at about week 12; (ix) inserting a fifth intravesical drug delivery system of the plurality of intravesical drug delivery systems into the bladder at about week 12; (x) removing the fifth intravesical drug delivery system at about week 15; (xi) inserting a sixth intravesical drug delivery system of the plurality of intravesical drug delivery systems into the bladder at about week 15; (xii) removing the sixth intravesical drug delivery system at about week 18; (xiii) inserting a seventh intravesical drug delivery system of the plurality of intravesical drug delivery systems into the bladder at about week 18; (xiv) removing the seventh intravesical drug delivery system at about week 21; (xv) inserting an eighth intravesical drug delivery system of the plurality of intravesical drug delivery systems into the bladder at about week 21; and (xvi) removing the eighth intravesical drug delivery system at about week 24, wherein each intravesical drug delivery system comprises about 225 mg of gemcitabine.

184. The method of embodiment 183, wherein inserting the intravesical drug delivery system into the bladder of the individual about once every 12 weeks (Q12W) for up to the additional 18 months after the first six months of treatment comprises the following steps: (xvii) inserting a ninth intravesical drug delivery system of the plurality of intravesical drug delivery systems into the bladder at about week 36; (xviii) removing the ninth intravesical drug delivery system at about week 39; (xix) inserting a tenth intravesical drug delivery system of the plurality of intravesical drug delivery systems into the bladder at about week 48; (xx) removing the tenth intravesical drug delivery system at about week 51; (xxi) inserting an eleventh intravesical drug delivery system of the plurality of intravesical drug delivery systems into the bladder at about week 60; (xxii) removing the eleventh intravesical drug delivery system at about week 63; (xxiii) inserting a twelfth intravesical drug delivery system of the plurality of intravesical drug delivery systems into the bladder at about week 72; (xxiv) removing the twelfth intravesical drug delivery system at about week 75; (xxv) inserting a thirteenth intravesical drug delivery system of the plurality of intravesical drug delivery systems into the bladder at about week 84; (xxvi) removing the thirteenth intravesical drug delivery system at about week 87; (xxvii) inserting a fourteenth intravesical drug delivery system of the plurality of intravesical drug delivery systems into the bladder at about week 96; and (xxviii) removing the fourteenth intravesical drug delivery system at about week 99, wherein each intravesical drug delivery system comprises about 225 mg of gemcitabine.

185. The method of any one of embodiments 94-184, wherein the total of up to six months ends with removal of an intravesical drug delivery system at about six months.

186. The method embodiment 184, wherein the additional 18 months after the first six months of treatment ends with removal of an intravesical drug delivery system at about 18 months following starting treatment.

187. The method of any one of embodiments 94-186, wherein each intravesical drug delivery system comprises about 256 mg of gemcitabine HCL.

### EXAMPLES

The following examples are exemplary and are not intended to limit the scope of any invention or inventions described herein.

### Example 1: In Vitro Release Rate of Gemcitabine Following Administration of TAR-200

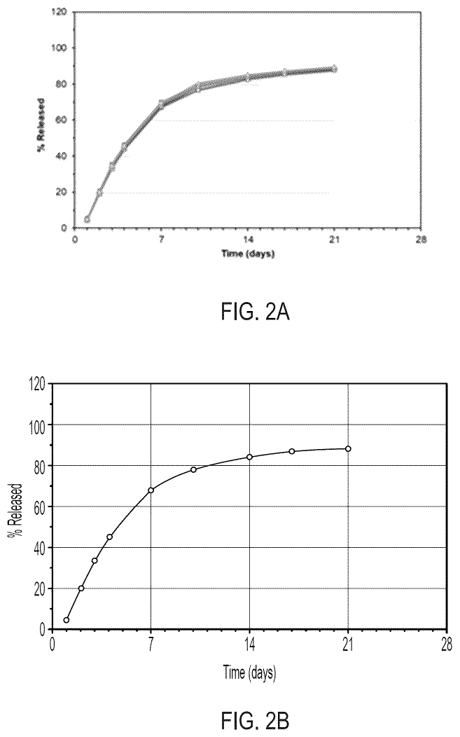

FIG. 2A-2B depict the percent of gemcitabine released from the TAR-200 intravesical drug delivery system in vitro. The intravesical drug delivery system containing 225 mg gemcitabine (256 mg gemcitabine HCL) was placed in 300 mL ultrapure water at 37 degrees C. 1 mL samples were taken at 1, 2, 3, 4, 7, 10, 14, 17, and 21 days after the device was placed in water. Samples were then analyzed using an Acquity UPLC® (CSH C18 1.7-μm particle size, 50×2.1 mm i.d.) to quantify gemcitabine released from the intravesical device. The results suggest that approximately 70% of the gemcitabine was released from the intravesical device by day 7, and that approximately 90% of the gemcitabine was released by day 21.

### Example 2: Urine Concentrations of Gemcitabine in Minipigs Following Administration of TAR-200

To obtain information on the metabolism, excretion, and plasma exposure of gemcitabine, female Göttingen minipigs received single intravesical administration of gemcitabine using TAR-200 on Day 0 followed by a single slow bolus intravesical administration of approximately 170 μCi (0.8 mg) 14C-gemcibatine on Day 2, that was retained in the bladder for 3 h by means of a balloon catheter. At all sampling time points (1, 4, 6 and 24 h post-dosing of radioactive gemcitabine or 49, 52, 54 and 72 h post TAR-200 dosing) traces of both gemcitabine and dFdU were present in plasma (average concentration ranged from 0.71 to 2.74 ng/ml and 6.70 to 16.8 ng/mL, respectively). No significant absorption of radioactivity was measured, and the majority of the administered radioactivity was excreted in urine over the 3 to 6 h urine time interval, after removal of the balloon catheter at 3 h post-dose. In the 0-120 h period post-dose, on average 89% of the radioactivity dose was excreted in urine, 7% in cage wash and cage materials, and only 1% in feces. Gemcitabine and the deaminated metabolite dFdU were the only entities detected in minipig urine following dosing with TAR-200.

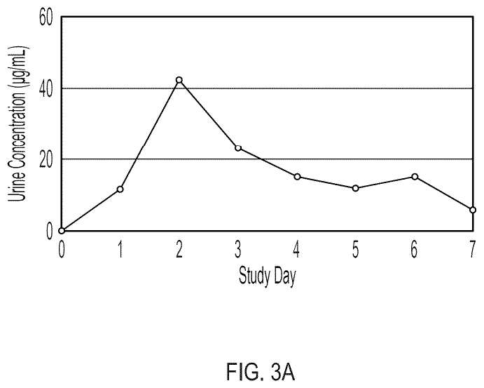

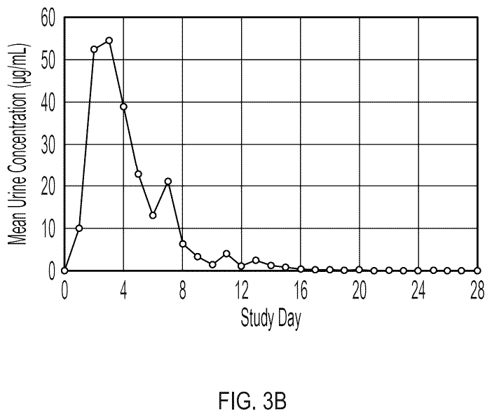

Average urine concentrations of at least 10 μg/mL are generally achieved by 24 hours and remain above that level through 168 hours. The maximum individual gemcitabine urine concentration in minipigs receiving 1 system ranged between 17.8 and 82.6 μg/mL, average of 42.4 μg/mL (n=9, GLP Tox study) and occurred between 2 and 7 days after insertion. The median time to maximum urine concentration ranged between 2 and 3 days (FIG. 3A). Additionally, intravesical gemcitabine did not produce meaningful systemic exposure since there were generally no quantifiable gemcitabine plasma concentrations (lower limit of quantitation=0.1 μg/mL) observed over the treatment period.

Gemcitabine tri- and mono-phosphate urine concentrations were reported below the limit of quantification with lower limits of quantification of 5 and 50 ng/ml respectively, in samples collected at day 2, 7 and 14 post TAR-200 insertion.

Use of TAR-200 for up to 21 days is based on the biphasic release properties observed in vitro and confirmed by in vivo minipig studies. In minipigs, during the first 7-day period, approximately 70% to 80% of the total gemcitabine content in TAR-200 is released, with <20% being released over the following 14-day period (FIG. 3B).

### Example 3: Urine Concentrations of Gemcitabine in Individuals Following Administration of TAR-200

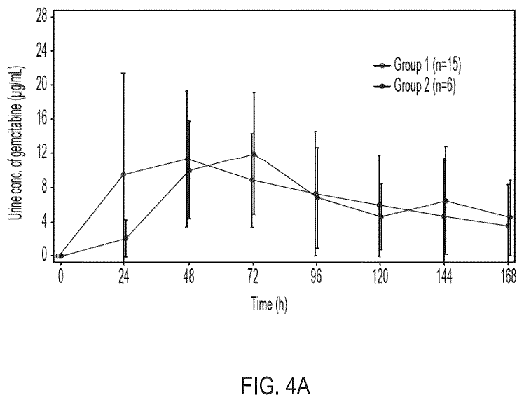

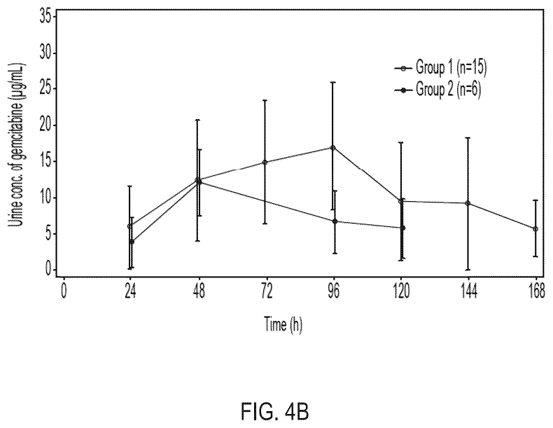

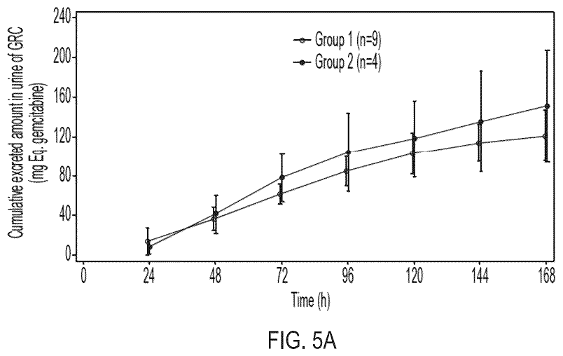

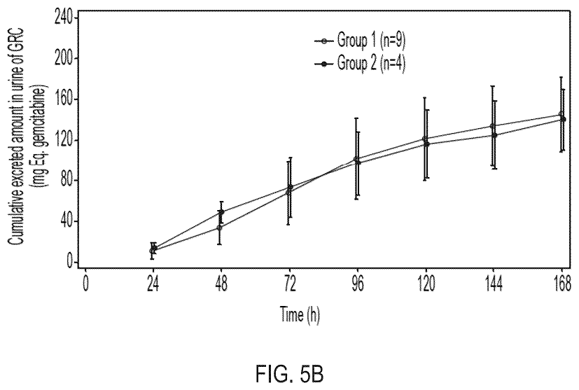

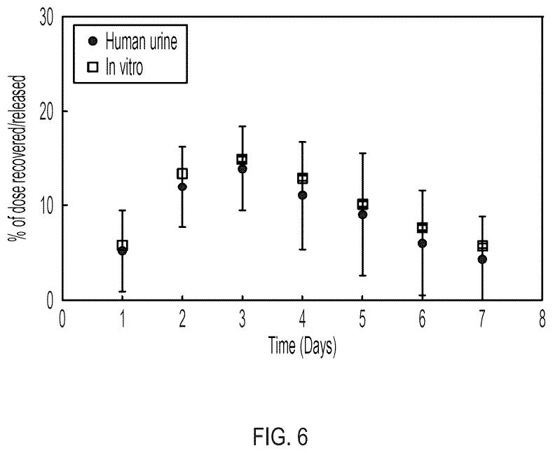

Pharmacokinetics of gemcitabine in plasma and urine were measured in individuals receiving TAR-200 (225 mg gemcitabine) (Group 1) or TAR-200 in combination with cetrelimab (360 mg) (Group 2) for two or more three-week dosing cycles of gemcitabine. After administration of gemcitabine via the TAR-200 intravesical drug delivery system, maximum gemcitabine concentration in urine was reached at a median Tₘₐₓ of 2 to 4 days and appeared to be similar for TAR-200+cetrelimab and TAR-200 alone. Mean gemcitabine concentration in urine exceeded the tumoricidal concentrations of 3 μg/mL (FIG. 4A-4B). Mean cumulative excreted gemcitabine in urine expressed as percentage of the administered dose from 0-168 hours ranged between 36.2-53.8%. Meanwhile, maximum dFdU concentration in urine was reached at median Tₘₐₓ between 3 and 6 days. Mean cumulative excreted dFdU amount in urine expressed as percentage of the administered dose from 0-168 hours ranged between 11.1-23.3%. The total excreted gemcitabine and dFdU was further converted to GRC in mg-equivalents gemcitabine (mg-Eq. gemcitabine) and are calculated as follows: GRC (mg-Eq. gemcitabine)=Gemcitabine in mg+dFdU in mg*(263.20/264.18). Mean cumulative excreted GRC amount in urine expressed as percentage of the administered dose from 0-168 hours was approximately 60% which appeared to be comparable for Group 1 and Group 2 for all weeks (FIG. 5A-5B). Plasma gemcitabine concentrations of all available samples were below the limit of quantitation [LOQ 0.1 μg/mL] which confirms that there is no systemic exposure of gemcitabine. Plasma dFdU concentrations of all except 2 samples were below the limit of quantitation [LOQ 0.1 μg/mL] which confirms that there is negligible systemic exposure of dFdU (≤0.397 μg/mL).

This example demonstrates that TAR-200 continuously releases gemcitabine into the urine at concentrations sufficient to treat tumors over a period of at least seven days or more supports use of TAR-200 in a three-week dosing cycle to treat bladder cancer.

### Example 4: a Phase 3 Study to Evaluate the Efficacy and Safety of Intravesical TAR-200 in Combination with IV Cetrelimab or TAR-200 Alone Versus Intravesical Bacillus Calmette-Guérin (BCG) in Participants with BCG-Naïve HR-NMIBC

The following example describes a Phase 3, Open-Label, Multi-Center, Randomized Study Evaluating the Efficacy and Safety of TAR-200 in Combination with Cetrelimab or TAR-200 Alone Versus Intravesical Bacillus Calmette-Guérin (BCG) in Participants with BCG-naïve High-Risk Non-Muscle Invasive Bladder Cancer (HR-NMIBC).

TAR-200 consists of gemcitabine minitablets (which may appear as solid material) and osmotic minitablets (for osmotic performance), and are contained within the bi-oval shaped device constituent. The color of the minitablets may vary but that has no effect on the potency of the TAR-200. The minitablets contain the following ingredients:

Gemcitabine minitablets: containing a total dose of 225 mg of the active ingredient gemcitabine (as free-base equivalent), and the following inactive ingredients: polyethylene glycol 8000 (8 mg), povidone K30 (13.4 mg), urea (42.6 mg).

Osmotic (urea) minitablets: contain no active ingredient, and the following inactive ingredients: urea (648 mg), polyethylene oxide 600,000 (72 mg), FD&C Blue No. 1 (0.0042 mg).

The bi-oval shaped tube consists of a dual lumen silicone part, a superelastic nitinol wire in a predefined shape (wireform), silicone spacers, and silicone adhesive. The minitablets are surrounded by a semipermeable membrane that contains a single delivery orifice. In some embodiments, the intravesical drug delivery system's coiled dimensions are approximately 5.5 cm wide×4.5 cm high.

TAR-200 is inserted into the bladder using a co-packaged device, the Urinary Placement Catheter, also known as “the Inserter” (hereafter referred to as “UPC”). The UPC is a sterile, single-use, transient contact medical device, which has been specifically developed for the transurethral placement of TAR-200 into the bladder. TAR-200 is loaded linearly into the clear shaft of the UPC and then inserted into the bladder by advancing the system through the clear shaft of the UPC with a stylet. TAR-200 returns to its original bi-oval form after exiting from the UPC when it is fully inserted into the bladder. TAR-200 is removed from the bladder transurethrally via cystoscopy and non-cutting endoscopic grasping forceps. An exemplary urinary placement catheter is described in U.S. Pat. No. 10,064,980, which is incorporated herein by reference in its entirety.

Cetrelimab is a fully human immunoglobulin G4 (IgG4) kappa monoclonal antibody containing the hinge stabilizing S228P mutation. Cetrelimab binds to programmed cell death protein 1 (PD 1) with high affinity and specificity, blocks binding to the programmed-cell death ligands 1 and 2 (PD L1 and PD-L2), enhances pro-inflammatory cytokine production from ex-vivo stimulated T cells, and reduces tumor volume in human PD-1 knock-in mice bearing MC38 murine colon carcinoma tumors.

### Overall Design

This is a multi-center, open-label, randomized Phase 3 study evaluating the efficacy and safety of intravesical TAR-200 in combination with IV cetrelimab or TAR-200 alone versus intravesical BCG in participants with BCG-naïve HR-NMIBC. High-risk is defined as high-grade papillary Ta, any T1 or CIS. All enrolled participants will have histologically confirmed high-grade papillary Ta, any T1 or CIS by local pathology.

Participants will be stratified based upon tumor stage (high-grade papillary Ta, any T1 or CIS) and tumor size (≤3 cm or >3 cm). All visible papillary disease must be fully resected (absent) prior to study treatment and documented at baseline cystoscopy. Participants will be randomly assigned 1:1:1 to treatment with either TAR-200+IV cetrelimab (Group A), TAR-200 alone (Group C), or intravesical BCG (Group B). The timing of the participant's HR NMIBC diagnosis should be within 90 days of the most recent signed informed consent. During the Screening period, immediate post-TURBT single-dose peri operative intravesical chemotherapy is allowed in accordance with institutional guidelines.

A target of 1050 participants will be randomly assigned (1:1:1) in this study with approximately 350 participants planned in each group.

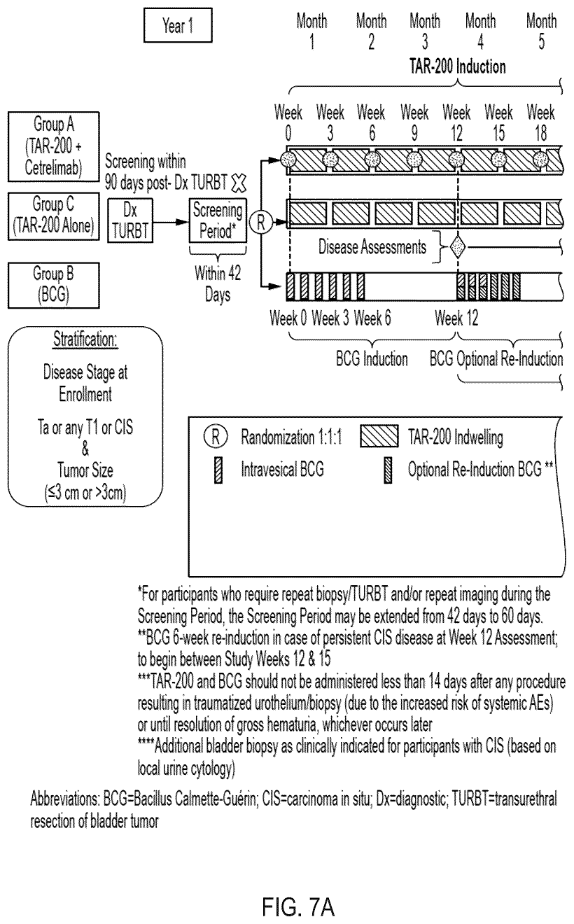

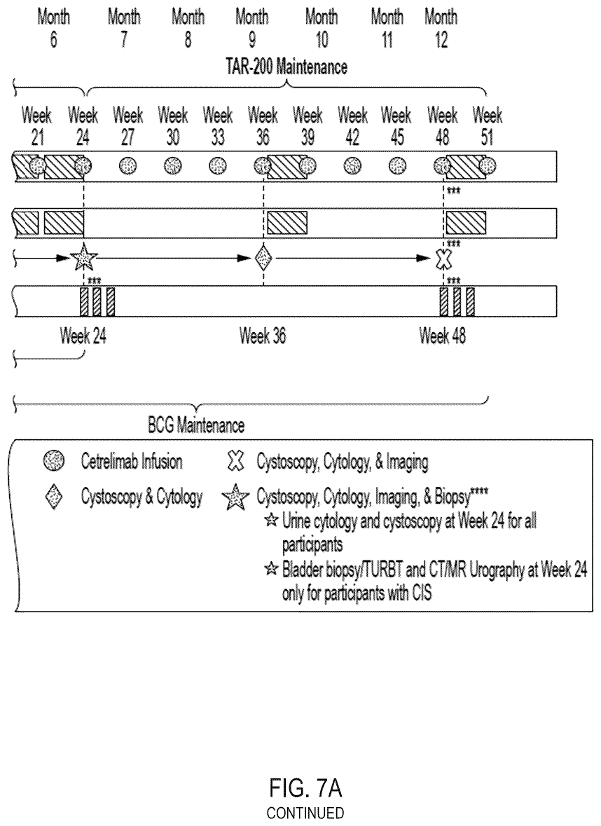

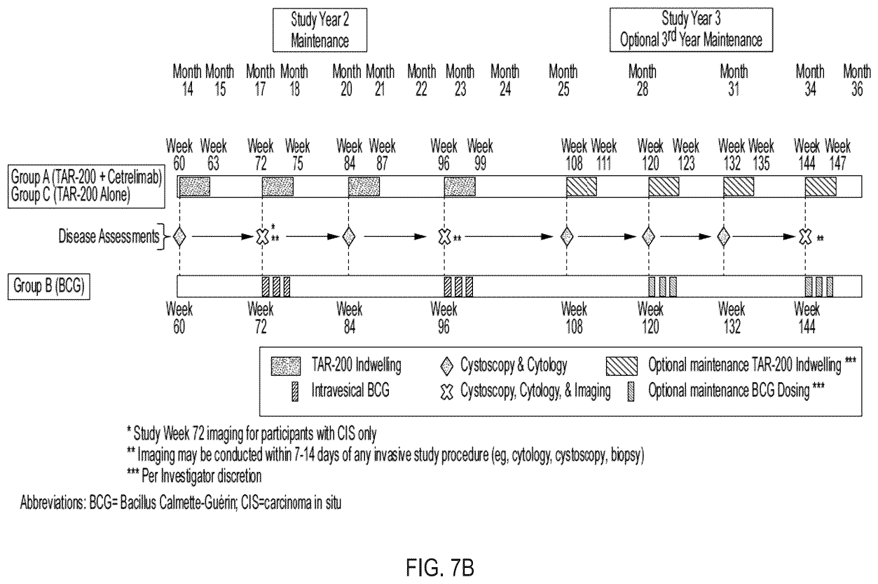

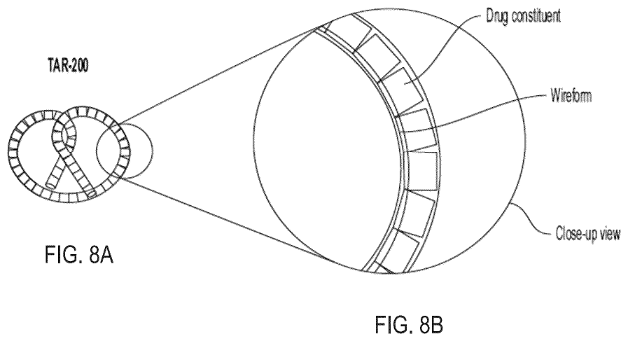

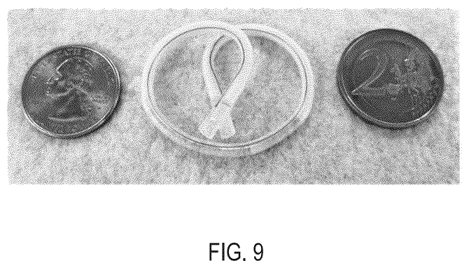

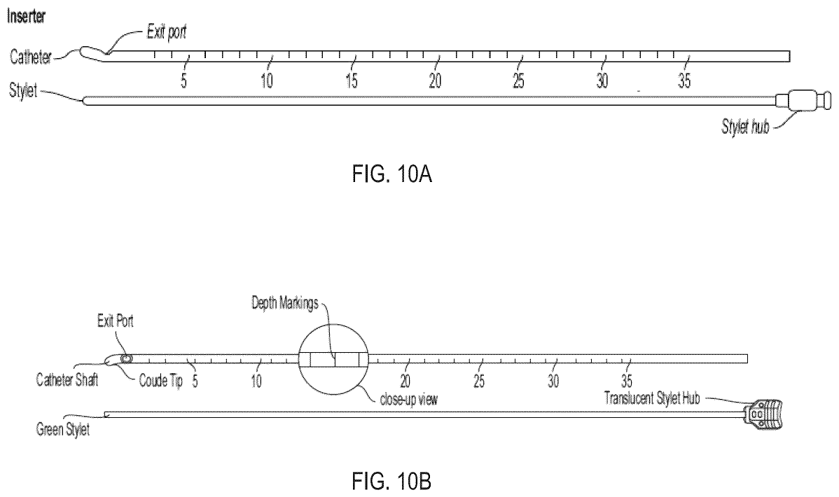

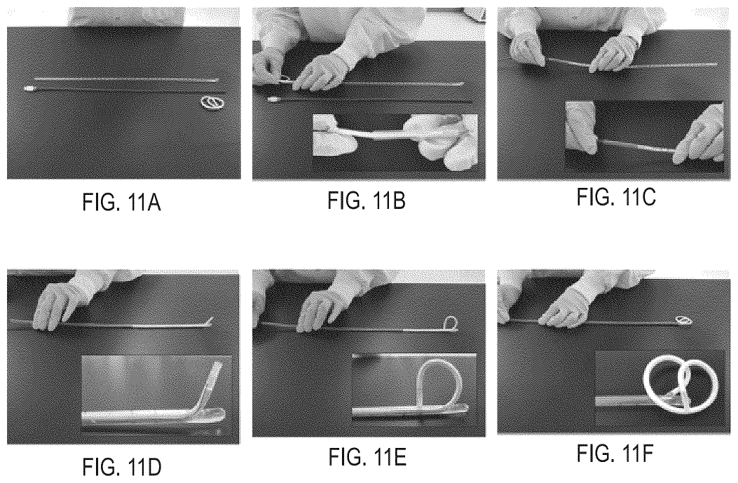

For participants in Groups A and C, TAR-200 will be dosed Q3W through Week 24 with TAR-200 insertion on Week 21 and removal on Week 24, and then every 12 weeks thereafter through Week 99 of Study Year 2 during the Treatment Phase. Dosing of TAR-200 for up to an additional year (Maintenance Treatment-Optional Year 3) will be optional at the discretion of the Investigator, and consists of dosing TAR-200 every 12 weeks (Week 108, through Week 147) with TAR-200 insertion on Week 144 and removal on Week 1476. (FIG. 7A-7B). As show in FIGS. 7A and 7B, TAR-200 is inserted in Weeks 0, 3, 6, 9, 12, 15, 18, and 21 through week 24 and in Weeks 36, 48, 60, 72, 84, and 96 beginning at week 24 through Week 99 (ending with removal of the last TAR-200 in week 99). Dosing of TAR-200 for up to an additional year (Optional Year 3), consisting of dosing of TAR-200 every 12 weeks, will be optional at the discretion of the Investigator (FIG. 7B). It is recommended that participants receive at least 1 dose of prophylactic periprocedural antibiotics at the time of TAR-200 insertion and/or removal.

For participants in Group A only, IV cetrelimab will be dosed at 360 mg IV Q3W through Week 51 (Study Year 1) during the Treatment Phase. (FIG. 7A). As shown in FIGS. 7A and 7B, cetrelimab is administered in weeks 0, 3, 6, 9, 12, 15, 18, 21, 24, 27, 30, 33, 36, 39, 42, 45, 48, and 51.

For participants in Group B, BCG will be dosed once weekly for 6 weeks (Induction) and then once weekly for 3 weeks starting at Weeks 12 (with optional re-induction for participants with persistent CIS disease at Week 12), 24, 48, 72 and 96 (Maintenance). (FIG. 7A-7B). Dosing of BCG for up to an additional year of maintenance treatment (Maintenance Treatment-Optional Year 3), consisting of once weekly dosing for 3 weeks starting at Weeks 120 and 144 will be optional at the discretion of the Investigator (FIG. 7B).

In all groups, the Treatment Phase will be followed by a 2-year Follow up Phase.

All groups will receive a diagnostic TURBT before randomization and screening within 90 days of the diagnostic TURBT, wherein individuals will undergo cystoscopy, cytology, and imaging. All groups will undergo cystoscopy, cytology, and imaging at about weeks 24, 48, 96, and 144 following start of treatment. All groups will undergo cystoscopy and cytology at about weeks 12, 36, 60, 72, 84, 108, 120, and 132 following start of treatment. For participants with CIS, bladder biopsy/TURBT and CT/MR Urography will be conducted at week 24, and imaging will be conducted at week 72. All assessments and treatment with the exception of Week 0 are to be completed within +1 Week. Maintaining a 3-week schedule is strongly recommended.

### Endpoints

### Primary Endpoints

Event-free survival (EFS) will be measured as the time from randomization to either the time of the first recurrence of high-risk disease, progression, or death due to any cause, whichever occurs first. For participants with CIS, persistent disease at 6 months (Week 24) is also considered an EFS event. Progression is defined as:
1) an increase of stage from Ta to T1 or from CIS to T1, or
2) progression to MIBC (T≥2) or to lymph node (N+) or to distant disease (M+), whichever occurs first.

All participants (participants with CIS and participants with papillary disease [high-grade Ta, any T1] in Groups A, B, and C), will be evaluated for efficacy in conjunction with disease assessments e.g., cystoscopy and local urine cytology, every 12 weeks (±1 week [Year 1] or ±2 weeks [Year 2 and Year 3]) during the Treatment Phase and through the first 5 visits of the Surveillance Follow-up Phase and then every 24 weeks until recurrence or progression.

The EFS endpoint will be determined by a combination of local and/or central disease assessments including local cytology, local cystoscopy, central pathology, or central imaging as applicable. An EFS event will be defined by 1) a positive local urine cytology, or 2) a positive local cystoscopy with confirmatory centrally reviewed biopsy proven high-risk disease (high-grade papillary Ta, any T1 or CIS disease), or 3) a centrally reviewed imaging proven disease progression/metastasis as applicable.

Participants are required to discontinue from study treatment if there is confirmed high-risk disease persistence (participants with CIS only), high-risk recurrence, or progression based on local urine cytology, local cystoscopy, central pathology, or central imaging as applicable after consultation with the Sponsor.

Participants with CIS

In all treatment groups, participants with CIS will be evaluated for persistent disease at the Week 12 assessment by cystoscopy and/or local urine cytology and/or central pathology. Participants in Group B with persistent CIS at the Week 12 assessment are eligible for a 6-week BCG re induction at the Investigator's discretion (where re induction is not clinically indicated, participants should proceed to Maintenance treatment). Participants in Group A and Group C with persistent CIS at the Week 12 assessment will remain on study treatment. Decisions for study treatment disposition for participants in all treatment groups with persistent CIS should not be determined until the Week 24 assessment. Those participants in all treatment groups with persistent CIS at Week 24 are required to discontinue study treatment as this is considered an EFS event. Participants enrolled with CIS in all treatment groups who continue onto maintenance treatment (i.e., are not discontinued due to persistent disease) and who later exhibit high-risk disease should be discontinued from study treatment as this is considered an EFS event.

### Secondary Endpoints

Overall complete response (CR) rate will be measured by determining the proportion of participants with CIS who have no presence of high-risk disease at 6 months. Duration of CR is defined from the time of first CR achieved to first evidence of recurrence, progression or death due to any cause (whichever occurs first) for participants who achieve a CR.

Recurrence-free survival (RFS) will be measured as the time from randomization to the time of the first recurrence of high-risk disease, or death due to any cause, whichever occurs first.

Time to progression (TTP) will be measured as the time from randomization to the date of first documented evidence of disease progression or death due to disease progression, whichever occurs first.

Overall survival (OS), defined as the time from randomization to death, due to any cause.

Cancer specific survival (CSS) will be measured as the time from randomization to the date of death due to bladder cancer.

To assess the safety and tolerability, frequency and grade of AEs (according to Common Terminology Criteria for Adverse Events [CTCAE] version 5.0). Laboratory abnormalities: CTCAE grades comparing baseline to the worst post-baseline value; other safety data, such as vital signs, will be considered as appropriate.

To compare health-related quality of life (HRQOL), change from baseline and time to symptom deterioration in European Organization for Research and Treatment of Cancer-Non-muscle Invasive Bladder Cancer 24 (EORTC-QLQ-NMIBC24).

### Exploratory Endpoints

Exploratory Endpoints include:
Incidence of and the time to cystectomy (TTC)
Metastasis-free survival (MFS)
To evaluate healthcare resource utilization and hospitalizations
All-cause event-free survival (aEFS)
To identify biomarkers in urine and tissue samples that are associated with EFS, expression of immune markers (PD-L1) by IHC or alternate methods
Characterization of determinants of response, innate and acquired resistance in tissue and urine
To evaluate the PK of gemcitabine and immunogenicity of cetrelimab in serum when IV cetrelimab is administered in combination with intravesical TAR-200 (Group A), serum cetrelimab concentration and incidence of anti-cetrelimab antibodies.
To assess EFS2 during the first subsequent non-surgical anticancer treatment. EFS2 is defined as the time from randomization until the date of recurrence of high risk disease, progression, or death due to any cause on the first subsequent non-surgical anticancer treatment, whichever occurs first.
To measure HRQOL, change from baseline and time to symptom deterioration in European Organization for Research and Treatment of Cancer-Quality of Life Questionnaire-Core 30 (EORTC-QLQ-C30) and EuroQol 5 Dimension 5 Level (EQ-5D-5L). Patient Global Impression Severity (PGIS) and Patient Global Impression of Change (PGIC) are selected anchors to interpret meaningful within person score change in PRO endpoints.

### Justification for Dose

### TAR-200 Dose Selection (Groups A and C)

TAR-200 is available in 1 dose only (comprising 225 mg of gemcitabine); and will remain indwelling for up to 3 weeks per each dosing cycle. TAR-200 will be dosed Q3W for the first 6 months on study (through Week 24) with TAR-200 insertion on Week 21 and removal on Week 24, and then every 12 weeks thereafter through Week 99 of Study Year 2 during the Treatment Phase. Dosing of TAR 200 for up to an additional year (Maintenance Treatment-Optional Year 3), will be optional at the discretion of the Investigator, and consists of dosing TAR-200 every 12 weeks (Week 108, through Week 147) with TAR-200 insertion on Week 144 and removal on Week 147.

Each TAR-200 releases gemcitabine into the bladder during the indwelling period, with the majority being delivered within the first 7 days. TAR-200 maintains therapeutic gemcitabine urine concentrations, exceeding the EC90 of many bladder tumor cell lines, in the bladder urine over the first 5 to 10 days (Jeon et al. J Urol. 2011; 186(5):2084-2093). Studies have demonstrated that prolonged gemcitabine exposure increases intracellular concentrations of the active di- and tri-phosphorylated metabolites enhancing overall drug potency compared to short duration dosing (Cattel et al. Ann Oncol. 2006; 17(5):142-147).

The first dosing regimen evaluated as part of Phase 1b study TAR-200-101 consisted of two 7-day dosing cycles with a 14-day recovery period in between dosing cycles. This regimen was found to be well tolerated by the study participants. To allow the remaining amount of drug (20% to 30%) in the system to continue to release into bladder urine, the indwelling period was later extended from 7 to 21 days (3 weeks). In addition to potentially improving efficacy, extension of the indwelling period allowed for removal of the previous system and placement of a new system to occur on the same visit, reducing the number of office visits for the participant. This additional residence time is not expected to result in a significant difference in terms of tolerability as the concentration of gemcitabine remaining in the TAR-200 after 7 days is low, and the rate of release of additional gemcitabine at Day 7 is minimal. The safety of (21-day indwelling) 3-week TAR-200 dosing cycles has been evaluated in 2 Phase 1b clinical studies (TAR-200-103 and TAR-200-104) and has been generally well tolerated to date based on data from these studies.

As there is only 1 dose option for TAR-200, there will be no dose modifications of TAR-200.

### TAR-200 Treatment Duration (Groups A and C)

The study protocol is designed to offer Year-2 therapy across the 3 arms, with the option of a Year-3 in all arms, in accordance with guideline recommendations and at Investigator's discretion.

The frequency of TAR-200 administration in Groups A and C decreases during the 2-year treatment period. During the first period, TAR-200 is administered every 3 weeks for 24 weeks. Subsequently, TAR-200 is administered once every 12 weeks until the last administration on Week 96, with the option of a third year at the discretion of the Investigator (with last administration on Week 144). During the 2-year treatment period, participants in Groups A and C are expected to receive 14 administrations of TAR-200 compared with 21 BCG instillations for Group B. Therefore, due to the indwelling nature of TAR-200, a smaller number of TAR-200 treatment insertions in Groups A and C is sufficient to ensure sustained or extended exposure to the active ingredient and to maintain a comparable treatment duration to the BCG control arm.

### Cetrelimab Dose Selection (Group A Only)

The recommended monotherapy dose for IV cetrelimab is based on clinical activity in the Phase 1 Study 63723283LUC1001. The identified recommended phase II dose (RP2D) regimens of IV cetrelimab are 240 mg Q2W and 480 mg Q4W administered intravenously. The IV cetrelimab monotherapy RP2D regimen selection was based on an acceptable safety profile and preliminary evidence that target concentrations are achieved.

For this study, IV cetrelimab will be dosed at 360 mg Q3W from Week 0 through Week 51. Dosing will align with the dosing frequency of TAR-200 through Week 24 and then continue Q3W through Week 51 (coinciding with TAR-200 dose administrations only on Week 36 and Week 48). The Q3W regimen is supported by the population PK modeling and simulation of cetrelimab, which demonstrated that the steady-state C_{trough} and Cₘₐₓ of cetrelimab attained is between the values from the two established RP2Ds (240 mg Q2W and 480 mg Q4W).

Dosing of IV cetrelimab may be delayed or discontinued depending on evaluation of participant laboratory results and general physical status.

### Cetrelimab Treatment Duration (Group A Only)

The addition of cetrelimab systemic therapy to TAR-200 is intended to augment anti-tumor activity of gemcitabine released by TAR-200 and to prevent immune evasion, resulting in a potentially more robust and durable response for this combination than that achieved with intravesical BCG.

### BCG Vesiculture Dose Selection (Group B)

All patients with HR-NMIBC should receive TURBT followed by full dose induction intravesical BCG treatment once weekly for 6 weeks.

For this study, a single dose of BCG Vesiculture will consist of 4 vials corresponding to 1 14×10⁸ CFU per instillation, comprising a full recommended dose of BCG. BCG will be dosed once weekly for 6 weeks (Induction) and then once weekly for 3 weeks at Weeks 12, 24, 48, 72 and 96 (Maintenance) (Study Year 2). Dosing of BCG for an additional year will be optional at the discretion of the Investigator.

No continued access will be proposed for this study.

### Study Population

### Inclusion Criteria

Each potential participant must satisfy all of the following criteria to be enrolled in the study:

### Age

1. Age ≥18 years (or the legal age of consent in the jurisdiction in which the study is taking place) at the time of informed consent.

### Disease Characteristic

2. Histologically confirmed initial diagnosis by local pathology (within 90 days of the most recent signed informed consent) of HR-NMIBC (high-grade Ta, any T1 or CIS), (AJCC, Cancer Staging Handbook Seventh Edition, 2017), in participants who are BCG-naïve. Mixed histology tumors are allowed if urothelial differentiation (transitional cell histology) is predominant. However, the presence of neuroendocrine, micropapillary, signet ring cell, plasmacytoid, or sarcomatoid features will make a participant ineligible.
   Participants may have had a history of HR-NMIBC (defined as high-grade Ta, any T1, or CIS) as long as it has been >3 years from current/novel diagnosis of HR-NMIBC (high-grade Ta, any T1 or CIS).
3. BCG-naïve (participants who have not received prior intravesical BCG or who previously received but stopped BCG more than 3 years before date of randomization are eligible)
4. Participants must be willing to undergo all study procedures (e.g., multiple cystoscopies from Screening through the end of study and TURBT/bladder biopsy for assessment of recurrence/progression).
5. All visible papillary disease must be fully resected (absent) prior to date of randomization and documented at baseline cystoscopy. Local urine cytology at screening must be negative or atypical (for HGUC) for patients with papillary only disease (without CIS).
6. All AEs associated with any prior surgery and/or intravesical therapy must have resolved to CTCAE version 5.0 Grade <2 prior to date of randomization.

### Type of Participant

7. Eastern Cooperative Oncology Group (ECOG) performance status Grade 0, 1, or 2.
8. Thyroid function tests within normal range or stable per Investigator assessment. Investigators may consult an endocrinologist for participant eligibility assessment in the case of equivocal or marginal tests results.
9. Adequate bone marrow, liver, and renal function:
   A. Bone marrow function (without the support of cytokines or erythropoiesis-stimulating agent in the preceding 2 weeks):

$$Absolute neurophil count \geq 1 , 000 / {mm}^{3} . \qquad i$$

$$Platelet count \geq 75 , 000 / {mm}^{3} . \qquad ii$$

$$Hemoglobin \geq 8. g / dL . \qquad iii$$

B. Liver function:
   i. Total bilirubin ≤1.5×ULN OR direct bilirubin ≤ULN for participants with total bilirubin levels >1.5×ULN (except participants with Gilbert's Syndrome, who must have a total bilirubin <3.0 mg/dL),
   ii. Alanine aminotransferase (ALT) and aspartate aminotransferase (AST) ≤2.5×institutional ULN
C. Renal function:

$$Creatinine clearance \geq 30 mL / min using the Cockcroft - Gault formula . \qquad i$$

### Exclusion Criteria

Any potential participant who meets any of the following criteria will be excluded from participating in the study:

### Disease Characteristics

1. Presence or history of histologically confirmed, muscle invasive, locally advanced, nonresectable, or metastatic urothelial carcinoma (i.e., ≥T2).
2. Must not have had urothelial carcinoma or histological variant at any site outside of the urinary bladder (i.e., urethra, ureter, or renal pelvis). Ta/any T1/CIS of the upper urinary tract (including renal pelvis and ureter) is allowable if treated with complete nephroureterectomy more than 24 months prior to randomization.
3. N+ and/or M+ per blinded independent central review (BICR) of computed tomography/magnetic resonance (CT/MR) Urography and chest CT.
   Any history of HR-NMIBC (high-grade Ta, any T1 or CIS)<3 years from current diagnosis.

### Medical Conditions

4. Active malignancies (i.e., progressing or requiring treatment change in the last 24 months prior to randomization) other than the disease being treated under study. Potential allowed exceptions include the following (others may be allowed with Sponsor approval).
   a. skin cancer (non-melanoma or melanoma) that is considered completely cured.
   b. non-invasive cervical cancer treated that is considered completely cured.
   c. adequately treated lobular carcinoma in situ (LCIS) and ductal CIS
   d. history of localized breast cancer and receiving antihormonal agents
   e. history of localized prostate cancer (NOMO) and receiving androgen deprivation therapy
   f. Localized prostate cancer (NOMO):
      i. with a Gleason score of 6, treated within the last 24 months or untreated and under surveillance,
      ii. with a Gleason score of 3+4 that has been treated more than 6 months prior to full study Screening and considered to have a very low risk of recurrence,
      iii. or history of localized prostate cancer and receiving androgen deprivation therapy and considered to have a very low risk of recurrence.
5. Presence of any bladder or urethral anatomical feature (e.g., urethral stricture) that, in the opinion of the Investigator, may prevent the safe insertion, indwelling use, removal of TAR-200 or administration of intravesical BCG. Participants with tumors involving the prostatic urethra in men will be excluded.
6. A history of clinically significant polyuria with recorded 24-hour urine volumes greater than 4000 mL.
7. Received a live virus vaccine within 30 days prior to the initiation of study treatment. Inactivated (non-live or non-replicating) vaccines approved or authorized for emergency use (e.g., COVID-19) by local health authorities are allowed.
8. Participants should not have a history of acute ischemic heart disease within 42 days of randomization, or history of uncontrolled cardiovascular disease including any of the following in the 3 months prior to randomization:
   a. unstable angina,
   b. myocardial infarction,
   c. ventricular fibrillation,
   d. Torsades de Pointes,
   e. cardiac arrest, or known congestive New York Heart Association Class III-IV heart failure,
   f. cerebrovascular accident,
   g. transient ischemic attack, or
   h. pulmonary embolism or other venous thromboembolism in the 3 months prior to randomization.
9. Indwelling catheters are not permitted; however, intermittent catheterization is acceptable.
10. Participants must not have clinically significant liver disease that precludes participant treatment regimens prescribed on the study (including, but not limited to active viral, alcoholic, or other autoimmune hepatitis, cirrhosis, or inherited liver disease).
11. Active hepatitis B or C infection (for example, participants with history of hepatitis C infection but undetectable hepatitis C virus polymerase chain reaction (PCR) test and participants with history of hepatitis B infection with positive HBsAg antibody and undetectable PCR are allowed).
12. Human immunodeficiency virus (HIV) infection, unless the participant has been on a stable anti-retroviral therapy regimen for the last 6 months or more prior to randomization and has had no opportunistic infections and a CD4 count of >350 in the last 6 months.
13. Participants with congenital immunodeficiencies.
14. Evidence of radiographic features associated with pulmonary fibrosis/advanced interstitial lung disease (ILD) (pulmonary consult may be required by the Investigator) as determined by BICR of chest CT, medical history of pneumonitis/ILD, or active non-infectious pneumonitis/ILD.
15. Evidence of active tuberculin infection (e.g., positive Mantoux test).
16. Major surgery and/or not fully recovered within 4 weeks before first dose (TURBT is not considered major surgery).
17.

### Prior/Concomitant Therapy

18. Received any BCG treatment within 3 years of date of randomization.
19. Received serial intervening intravesical chemotherapy or immunotherapy from the time of pre-Screening (diagnostic) to date of randomization. Immediate post-TURBT single-dose peri-operative intravesical chemotherapy is allowed per institutional guidelines.
20. Prior therapy with PD-1, PD-L1 or PD-L2 inhibitors, or with an agent directed to another co-inhibitory T-cell receptor.
21. Participants with a history of prior disseminated BCG exposure (e.g., BCG-osis/sepsis) or Grade ≥3 toxic effects when using anti-TNF or anti-IL-6 agents are excluded.
22. Not recovered from toxicity of prior anti-cancer therapy (except toxicities which are not clinically significant such as alopecia, skin discoloration).
23. Participants who require immunosuppressive medications including but not limited to systemic corticosteroid at doses >10 mg/day of prednisone or its equivalence, methotrexate, cyclosporine, azathioprine, and TNF a blockers. Treatment with anti-tuberculosis medicines such as isoniazid, rifampicin, and ethambutol. Use of immunosuppressive medications for the management of immune-related AEs, infusion-related reactions, or in participants with contrast allergies is acceptable. Use of inhaled, topical, and intranasal corticosteroids are permitted.
24. Active autoimmune disease that has required systemic treatment in the 2 years prior to randomization.
25. Active infection requiring systemic IV therapy within 14 days prior to randomization (See exclusion criterion no. 26 for information regarding urinary tract infection [UTI]).
26. Concurrent urinary tract infection (UTI), defined as a symptomatic infection with a positive urine culture with a bacterial count of >105 colony forming units (CFU)/mL in urine voided from women, or >10⁴ CFU/mL in urine voided from men, or in straight-catheter urine from women that cannot be cleared with antibiotic therapy.
27. Participants with a history of allergy to protein-based therapies and participants with a history of any significant drug allergy (such as anaphylaxis, hepatotoxicity, or immune-mediated thrombocytopenia or anemia) are excluded.
28. Known hypersensitivity to any study component including:
   a. Gemcitabine (or other drug excipients) or chemically-related drugs,
   b. TAR-200 device constituent materials,
   c. UPC materials,
   d. Cetrelimab (or other drug excipients) or chemically-related drugs,
   e. BCG Vesiculture (or other drug excipients) or chemically-related drugs.
   Refer to the TAR-200 IB, cetrelimab IB, and BCG Vesiculture SmPC for complete information on excipients.

### Prior/Concurrent Clinical Study Experience

29. Currently participating or has participated in a study of an investigational agent and received study therapy or investigational device within 4 weeks prior to the date of randomization.

### Diagnostic Assessments

30. Evidence of bladder perforation during diagnostic cystoscopy.
31. Bladder post-void residual (PVR) volume >350-mL after second voided urine at Screening.

### Other Exclusions

32. The participant is unable to comply with the requirements of this protocol, including any factors that are likely to affect the participant's return for scheduled visits and follow-up.
33. Any condition for which, in the opinion of the Investigator, participation would not be in the best interest of the participant (eg, compromise the well-being) or that could prevent, limit, or confound the protocol-specified assessments.
34. A pyeloureteral tube externalized to the skin is exclusionary. Unilateral nephrostomy tube or ureteral stent is permitted if it does not interfere with placement or retention of TAR-200 in the bladder. Participants with unilateral hydronephrosis are permitted; however, participants with bilateral hydronephrosis are excluded.

### Pharmacokinetic and Immunogenicity

### For TAR-200 (Groups A and C)

One single collection of urine samples will be collected from participants within 24 hours pre-dose at Week 0 Visit. Two pooled 24-hr cumulative urine collections will be collected from participants. These sparse urine samples will be collected for assessment of gemcitabine and 2′,2′-difluorodeoxyuridine (dFdU) concentrations in urine.

Blood samples will be collected from participants at multiple timepoints which coincide with urine pharmacokinetic (PK) timepoints. These blood samples will be collected for assessment of gemcitabine and dFdU concentrations in plasma.

### For IV Cetrelimab (Group a Only)

Blood samples for PK and immunogenicity will be collected from participants at multiple timepoints.

### Safety Evaluations

Safety assessments will be based on medical review of adverse event (AE) reports and the results of vital sign measurements, 12-lead electrocardiogram (ECG), physical examinations, clinical laboratory tests, and other safety evaluations at specified timepoints.

All AEs will be collected from the time of the informed consent form signature to the end of the 100-day safety follow-up visit. All drug-related serious adverse events (SAEs) beyond 100 days after last dose of study drug(s) must be reported for participants during the Follow-up Phase. The Sponsor will evaluate any safety information that is spontaneously reported by an Investigator beyond the time frame specified in the protocol. Any clinically significant abnormalities or toxicities persisting at the end of the study/early withdrawal will be followed by the Investigator until resolution or until reaching a clinically stable condition.

Safety oversight will be performed by the study Sponsor's Medical Monitor and IDMC, which will be composed of individuals with the appropriate expertise. The IDMC will operate under an approved charter and will meet as outlined in the IDMC charter. An IDMC will review safety data periodically during the study in accordance with the IDMC charter.

### Example 5: Safety Results of Ongoing Phase 3 Study

Safety results were obtained from the study described in Example 4. Treatment-emergent adverse events (TEAEs) regardless of relationship were reported in 455 participants (73.4%). Of these, TEAEs reported in 218 participants (52.7%) were considered related to TAR-200. TAR-200 related TEAEs occurring in ≥5.0% of participants were dysuria (22.2%), pollakiuria (19.1%), micturition urgency (12.6%), hematuria (9.9%), urinary tract infection (UTI, 8.5%) and cystitis noninfective (6.0%).

Regardless of relationship, Grade ≥3 TEAEs were reported in 73 (11.8%) participants. Of these, TEAEs reported in 23 participants (5.6%) were considered related to TAR-200. By SOC and PT, there were no TAR-200 related Grade ≥3 TEAEs reported in >2% participants. SAEs were reported in 45 participants (7.3%) and were considered related to TAR-200 in 9 participants (2.2%). Regardless of relationship, there were no SAEs by system organ class (SOC) and preferred term (PT) reported in >2% participants.

A total of 25 participants (4.0%) discontinued treatment due to TEAEs, with TEAEs considered related to TAR-200 in 13 participants (3.1%). Regardless of relationship, there were no TEAEs leading to treatment discontinuation (by SOC and PT) reported in >2% participants. TEAEs leading to death were reported in 2 participants, none of these were related to TAR-200.

These results support the use of TAR-200 to deliver up to eighteen total doses of gemcitabine alone or in combination with cetrelimab to treat BCG-naïve non-muscle invasive bladder cancer.

### Example 6: Development of a Population PK Model

The primary objective of the Population PK (popPK) analysis was to develop a popPK model to characterize the PK and variability of urine GRC following administration of TAR-200.

The popPK analysis was performed using pooled data from individuals treated with TAR-200 in combination with cetrelimab and individuals treated with TAR-200 monotherapy from clinical trial NCT04640623. In total, there were 618 PK evaluable urine samples, out of which 23 (4%) were below quantification limit (BQL) for both gemcitabine and dFdU, and 177 (29%) were BQL for either gemcitabine or dFdU. Urine samples of both analytes or either analyte being BQL were excluded from popPK modeling. The popPK analysis included 416 urine samples from 97 participants.

The model development started with a first-order release model from TAR-200 and was updated to account for the delay of drug release. Inter-individual variability (IIV) on drug release parameters were modeled assuming a log-normal distribution except for the proportion of gemcitabine available for release (BIO) parameter, which was assumed to have a logit-normal distribution to restrain individual BIO between 0 and 1. The daily GRC amount data were modeled on the logarithmic scale with an additive residual error model (equivalent to a proportional error model on the normal scale). Model selection was based on model diagnostic plots and objective function value (OFV). PK simulations were performed to derive the 7-day cumulative urine GRC amount based on individual post-hoc parameters to support exposure subgroup analysis and exposure-response (E-R) analysis. Exposure across different subgroups such as age, sex, weight, race, ethnicity, and renal function were compared using model-predicted 7-day cumulative GRC amount and observed daily urine GRC and gemcitabine exposure (ie, gemcitabine concentration, gemcitabine amount, and GRC amount).

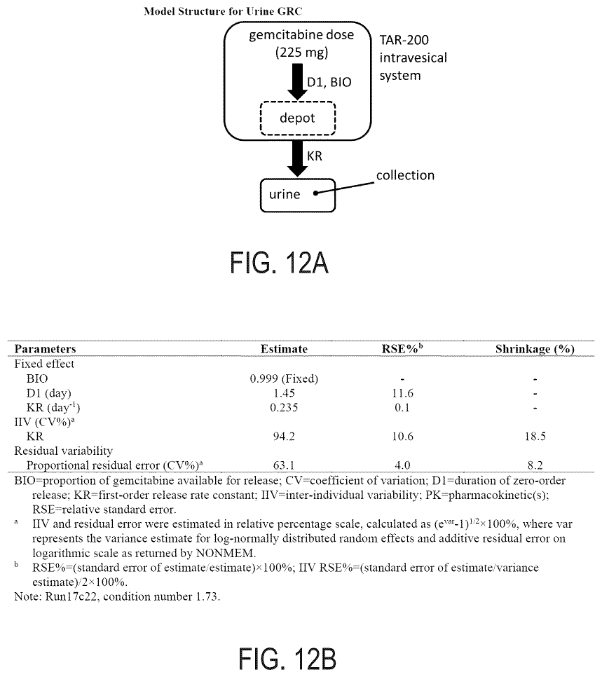

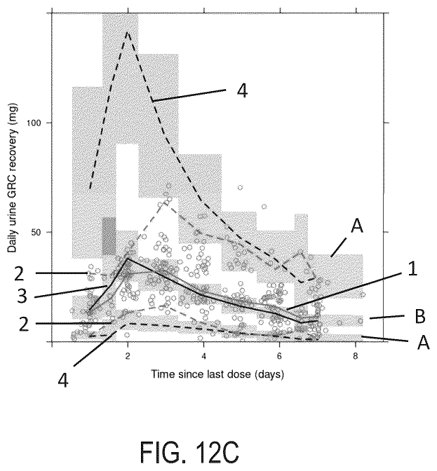

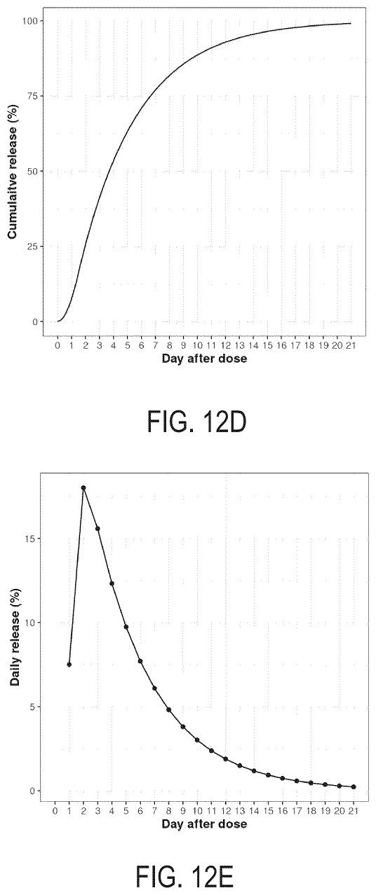

The release of gemcitabine from TAR-200 was adequately described by a sequential zero- and first-order release. The model structure was presented in FIG. 12A. The model was parameterized with proportion of gemcitabine available for release (BIO), duration of zero-order release (D1), and first-order release rate constant (KR). The parameter estimates of the final popPK model are summarized in FIG. 12B.

All model parameters were estimated with adequate precision with relative standard error (RSE)<12%. Because the study collected urine data up to Day 7 of the dosing cycle, resulting in incomplete information for the estimation of BIO, the population BIO was fixed at 0.999. This was supported by both an in vitro study and the post-use assessment of TAR-200 in minipig, which concluded that gemcitabine was nearly completely released by Day 21. IIV was quantified on KR. IIV of other release parameters such as D1, BIO was attempted but was not supported by the data (ie, model failed to converge). The residual variability was high, which could be because of incomplete collection of urine samples and/or incorrect collection time, measurement error, as well as model misspecification. The variability of urine GRC amount was largely attributed to residual unexplained variability and the IIV on KR.

Visual predictive check (VPC) indicates that the model was able to capture the general trend of the urine GRC amount over time (FIG. 12C) and the model tended to underpredict the daily urine GRC amount. The model appeared to overpredict the overall variability of urine GRC amount.

Based on the simulated typical profiles of cumulative release (FIG. 12D), 76.9% and 99.0% of gemcitabine were released by Day 7 and Day 21, respectively. The simulated cumulative release was consistent with the in vitro release profile (approximately 70% of gemcitabine released by Day 7, and approximately 90% by Day 21) and post-use assessment of TAR-200 in minipig, suggesting a reasonable model fit. Based on the simulated typical profile of daily release, 18.0% and 15.6% of gemcitabine was released on Day 2 and Day 3, respectively, with Day 2 being the peak release. This was generally consistent with the observed data (FIG. 12E).

### CONCLUSION

The release of gemcitabine from TAR-200 was characterized using a popPK model with sequential zero- and first-order release. A flat urine exposure-efficacy relationship for CR rate supports the choice of the 225 mg gemcitabine dose.

1-19. (canceled)

20. A method of treating Bacillus Calmette-Guérin (BCG)-naïve non-muscle invasive bladder cancer (NMIBC) in an individual, comprising inserting an intravesical drug delivery system comprising about 225 mg of gemcitabine into the bladder about once every 3 weeks up to 6 months (8 doses), followed by about once every 12 weeks for up to 2 years from first dose, or until persistent or recurrent NMIBC, disease progression, or unacceptable toxicity.

21. The method of claim 20, wherein the intravesical drug delivery system is administered as a monotherapy for treating Bacillus Calmette-Guérin (BCG)-naïve non-muscle invasive bladder cancer (NMIBC).

22. A method of treating Bacillus Calmette-Guérin (BCG)-naïve non-muscle invasive bladder cancer (NMIBC) in an individual, comprising:
inserting an intravesical drug delivery system comprising about 225 mg of gemcitabine into the bladder about once every 3 weeks up to 6 months (8 doses), followed by about once every 12 weeks for up to 2 years from first dose, or until persistent or recurrent NMIBC, disease progression, or unacceptable toxicity, and
administering cetrelimab for up to 1 year from first dose, or until persistent or recurrent NMIBC, disease progression, or unacceptable toxicity.

23. The method of claim 20, wherein each intravesical system is removed after a 3-week indwelling period.

24-26. (canceled)

27. A method of treating Bacillus Calmette-Guérin (BCG)-naïve non-muscle invasive bladder cancer (NMIBC) in an individual, comprising:
inserting an intravesical drug delivery system comprising about 225 mg of gemcitabine into the bladder of the individual about once every three weeks (Q3W) for a total of up to six months following starting treatment, followed by
inserting an intravesical drug delivery system comprising about 225 mg of gemcitabine into the bladder of the individual about once every 12 weeks (Q12W) for up to an additional 18 months after the first six months of treatment,
wherein each intravesical drug delivery system is removed from the bladder about three weeks after insertion into the bladder and wherein each intravesical drug delivery system is removed prior to insertion of another intravesical drug delivery system, and
wherein event-free survival (EFS) of the individual is extended compared to EFS achieved by intravesical BCG, and/or wherein EFS in a population of patients receiving such therapy is extended compared to a population of patients receiving intravesical BCG.

28-32. (canceled)

33. A method of treating Bacillus Calmette-Guérin (BCG)-naïve non-muscle invasive bladder cancer (NMIBC) in an individual, comprising:
intravenously administering to the individual about 360 mg of cetrelimab about once every three weeks (Q3W) for at least six months following starting treatment; and
inserting an intravesical drug delivery system comprising about 225 mg of gemcitabine into the bladder of the individual about once every three weeks (Q3W) for a total of up to six months following starting treatment, followed by
inserting an intravesical drug delivery system comprising about 225 mg of gemcitabine into the bladder of the individual about once every 12 weeks (Q12W) for up to an additional 18 months after the first six months of treatment,
wherein each intravesical drug delivery system is removed from the bladder about three weeks after insertion into the bladder and wherein each intravesical drug delivery system is removed prior to insertion of another intravesical drug delivery system, and
wherein event-free survival (EFS) of the individual is extended compared to EFS achieved by intravesical BCG, and/or wherein EFS in a population of patients receiving such therapy is extended compared to a population of patients receiving intravesical BCG.

34. The method of claim 33, wherein intravenously administering to the individual about 360 mg of cetrelimab about once every three weeks (Q3W) for at least six months comprises intravenously administering to the individual about 360 mg of cetrelimab about Q3W for about one year.

35-36. (canceled)

37. The method of claim 22, wherein the administration of the cetrelimab and the insertion of the intravesical drug delivery system occurs no more than 72 hours apart from each other.

38. The method of claim 22, wherein the administration of the cetrelimab and insertion of the intravesical drug delivery system occurs on the same day, and wherein the method comprises administering the intravesical drug delivery system at least 45 minutes prior to or after the completion of the administration of the cetrelimab.

39-40. (canceled)

41. The method of claim 20, wherein inserting another intravesical drug delivery system occurs on the same day as removing a previous intravesical drug delivery system within the first six months of treatment.

42. The method of claim 27, wherein inserting the intravesical drug delivery system into the bladder of the individual about once every 12 weeks (Q12W) for up to the additional 18 months begins at about week 36 after starting treatment, and wherein for a twelve-week period after the first six months of treatment, no intravesical drug delivery system is present in the bladder.

43. The method of claim 27, wherein inserting the intravesical drug delivery system into the bladder of the individual about once every three weeks (Q3W) for a total of up to six months comprises eight three-week dosing cycles of gemcitabine, and
wherein inserting the intravesical drug delivery system into the bladder of the individual about once every 12 weeks (Q12W) for up to an additional 18 months comprises up to six twelve-week dosing cycles of gemcitabine.

44. (canceled)

45. The method of claim 20, wherein the method comprises up to fourteen total dosing cycles of gemcitabine.

46. The method of claim 22, wherein intravenously administering the cetrelimab comprises eighteen dosing cycles of cetrelimab.

47. The method of claim 27, comprising, at about week 108 after start of treatment, inserting an intravesical drug delivery system comprising about 225 mg of gemcitabine into the bladder of the individual about once every 12 weeks (Q12W) for up to an additional ten months, wherein each intravesical drug delivery system is removed from the bladder about three weeks after insertion into the bladder and wherein each intravesical drug delivery system is removed prior to insertion of another intravesical drug delivery system.

48-50. (canceled)

51. The method of claim 47, wherein the method comprises up to eighteen total dosing cycles of gemcitabine.

52-53. (canceled)

54. The method of claim 20, comprising extending event-free survival (EFS) of the individual compared to the EFS achieved by intravesical BCG, and/or extending EFS in a population of patients receiving such therapy compared to a population of patients receiving intravesical BCG.

55. The method of claim 20, wherein the method results in:
(a) an increased overall complete response (CR) rate in a population of patients who have received treatment compared to the CR rate achieved by intravesical BCG;
(b) an increased duration of CR compared to the duration of CR achieved by intravesical BCG;
(c) an increased recurrence-free survival (RFS) compared to the RFS achieved by intravesical BCG;
(d) an increased time to progression (TTP) compared to the TTP achieved by intravesical BCG;
(e) an increased overall survival (OS) compared to the OS achieved by intravesical BCG;
(f) an increased cancer specific survival (CSS) compared to the CSS achieved by intravesical BCG;
(g) improved safety and tolerability compared to the safety and tolerability of intravesical BCG; and/or
(h) improved health-related quality of life (HRQOL) compared to the HRQOL of intravesical BCG.

56. The method of claim 20, wherein the individual has:
(a) high-grade Ta stage bladder cancer;
(b) T1 stage bladder cancer;
(c) carcinoma in situ (CIS);
(d) a mixed histology tumor with predominant urothelial differentiation;
(e) an Eastern Cooperative Oncology Group (ECOG) performance status Grade 0, 1, or 2; and/or
(f) papillary disease, and wherein the papillary disease is fully resected prior to treatment with gemcitabine.

57. The method of claim 20, wherein the individual does not have:
(a) a presence or history of histologically confirmed, muscle invasive, locally advanced, nonresectable, or metastatic urothelial carcinoma of stage T2 or greater;
(b) urothelial carcinoma or a histological variant at any site outside of the bladder;
(c) a history of NMIBC less than three years from current diagnosis;
(d) visible papillary disease at the time of diagnosis;
(e) N+ and/or M+ bladder cancer;
(f) neuroendocrine, micropapillary, signet ring cell, plasmacytoid, or sarcomatoid features;
(g) a presence of any bladder or urothelial anatomical feature that prevents the safe insertion, indwelling use, and removal of the intravesical drug delivery system; and/or
(h) a history of clinically significant polyuria with recorded 24-hour urine volumes greater than 4000 mL.

58. The method of claim 20, wherein the individual has not received BCG within three years from diagnosis.

59-61. (canceled)

62. The method of claim 20, wherein the intravesical drug delivery system is inserted into the bladder transurethrally using a catheter.

63. (canceled)

64. The method of claim 20, wherein the intravesical drug delivery system comprises a flexible, bi-oval shaped housing comprising a dual lumen,
said dual lumen comprising:
a drug reservoir lumen containing a first unit comprising gemcitabine minitablets and a second unit comprising an osmotic agent comprising urea minitablets; and
a retention frame lumen containing a predefined shaped super-elastic nitinol wire retention frame,
wherein the drug reservoir lumen comprises a single delivery orifice that controllably releases the gemcitabine from the drug reservoir lumen through the delivery orifice via osmotic pressure generated by the osmotic agent,
wherein the housing is elastically deformable between a retention shape configured to retain the intravesical drug delivery system in the individual's bladder and a deployment shape configured for passage of the intravesical drug delivery system through the individual's urethra.

65-70. (canceled)

71. The method of claim 64, wherein the first unit further comprises urea, wherein the first unit contains at least 75 percent by weight gemcitabine HCl; and/or wherein the second unit contains at least 85 percent by weight urea.

72-77. (canceled)

78. The method of claim 20, wherein the intravesical drug delivery system comprises an almost white to light pink-brown colored gemcitabine component at the center surrounded on each side by off white to light blue-colored osmotic components.

79. The method of claim 20, wherein the intravesical drug delivery system releases gemcitabine at a zero-order release rate for an extended period, followed by a reduced, non-zero-order release rate over a decay period.

80. The method of claim 20, wherein the concentration of gemcitabine in the urine of the individual is between about 4 μg/mL and about 50 μg/mL while the intravesical drug delivery system is inserted into the bladder.

81. The method of claim 20, wherein about 65% to about 85% of the gemcitabine in the intravesical drug delivery system is released into the urine of the individual by day seven after insertion into the bladder; and/or
wherein about 85% to about 100% of the gemcitabine in the intravesical drug delivery system is released into the urine of the individual by day 21 after insertion into the bladder.

82. (canceled)

83. The method of claim 20, wherein inserting the intravesical drug delivery system into the bladder of the individual about once every three weeks (Q3W) for a total of up to six months following starting treatment comprises the following steps:
(i) inserting a first intravesical drug delivery system of a plurality of intravesical drug delivery systems into the bladder at about week 0;
(ii) removing the first intravesical drug delivery system at about week 3;
(iii) inserting a second intravesical drug delivery system of the plurality of intravesical drug delivery systems into the bladder at about week 3;
(iv) removing the second intravesical drug delivery system at about week 6;
(v) inserting a third intravesical drug delivery system of the plurality of intravesical drug delivery systems into the bladder at about week 6;
(vi) removing the third intravesical drug delivery system at about week 9;
(vii) inserting a fourth intravesical drug delivery system of the plurality of intravesical drug delivery systems into the bladder at about week 9;
(viii) removing the fourth intravesical drug delivery system at about week 12;
(ix) inserting a fifth intravesical drug delivery system of the plurality of intravesical drug delivery systems into the bladder at about week 12;
(x) removing the fifth intravesical drug delivery system at about week 15;
(xi) inserting a sixth intravesical drug delivery system of the plurality of intravesical drug delivery systems into the bladder at about week 15;
(xii) removing the sixth intravesical drug delivery system at about week 18;
(xiii) inserting a seventh intravesical drug delivery system of the plurality of intravesical drug delivery systems into the bladder at about week 18;
(xiv) removing the seventh intravesical drug delivery system at about week 21;
(xv) inserting an eighth intravesical drug delivery system of the plurality of intravesical drug delivery systems into the bladder at about week 21; and
(xvi) removing the eighth intravesical drug delivery system at about week 24,
and wherein inserting the intravesical drug delivery system into the bladder of the individual about once every 12 weeks (Q12W) for up to an additional 18 months after the first six months of treatment comprises the following steps:
(xvii) inserting a ninth intravesical drug delivery system of the plurality of intravesical drug delivery systems into the bladder at about week 36;
(xviii) removing the ninth intravesical drug delivery system at about week 39;
(xix) inserting a tenth intravesical drug delivery system of the plurality of intravesical drug delivery systems into the bladder at about week 48;
(xx) removing the tenth intravesical drug delivery system at about week 51;
(xxi) inserting an eleventh intravesical drug delivery system of the plurality of intravesical drug delivery systems into the bladder at about week 60;
(xxii) removing the eleventh intravesical drug delivery system at about week 63;
(xxiii) inserting a twelfth intravesical drug delivery system of the plurality of intravesical drug delivery systems into the bladder at about week 72;
(xxiv) removing the twelfth intravesical drug delivery system at about week 75;
(xxv) inserting a thirteenth intravesical drug delivery system of the plurality of intravesical drug delivery systems into the bladder at about week 84;
(xxvi) removing the thirteenth intravesical drug delivery system at about week 87;
(xxvii) inserting a fourteenth intravesical drug delivery system of the plurality of intravesical drug delivery systems into the bladder at about week 96; and
(xxviii) removing the fourteenth intravesical drug delivery system at about week 99,
wherein each intravesical drug delivery system comprises about 225 mg of gemcitabine.

84-86. (canceled)

87. The method of claim 20, wherein each intravesical drug delivery system comprises about 256 mg of gemcitabine HCL.

88. The method of claim 20, wherein the non-muscle invasive bladder cancer is high-risk (HR-NMIBC).
